SEQUENCE LISTING (34 sequences)

SEQ ID NO: 1 - protein, 379 aa, Zea mays
MMMSKQMLVLSLLLVLFELGSLPTTSCKKVFFNLWVTNQPANATQDAGCAKKDDALSSADTIKVWNYIDPASQLRPEDGGYTTISESIANIPEDNAKRYLLILKPGVVFREKLLLGRSKPFITIMSEDPMNPAVIVWNDTATTMGKDGKPLGVDGSSTMAIESDYFVAYNVVFKNDAPLPKLGEKKGEALALRVMGTKATFYNCTIEGGQGALYDQTGLHYFKACAIKGTIDFIFGSAKSFYEECKIVSVLKEALALPLAPPEQDRSRNPIKIAPGKSGLAFKTCTIEGEGEKIYLGRVGTPVIYSYTNIGKEIVGIISNGQDVQTVERGYYCATFKCYGPGMSPMVTSTLTYVQAIPFLGIYYISGESWIPSLPPIEE

SEQ ID NO: 2 - protein, 356 aa, Zea mays
TTSCKKVFFNLWVTNQPANATQDAGCAKKDDALSSADTIKVWNYIDPASQLRPEDGGYTTISESIANIPEDNAKRYLLILKPGVVFREKLLLGRSKPFITIMSEDPMNPAVIVWNDTATTMGKDGKPLGVDGSSTMAIESDYFVAYNVVFKNDAPLPKLGEKKGEALALRVMGTKATFYNCTIEGGQGALYDQTGLHYFKACAIKGTIDFIFGSAKSFYEECKIVSVLKEALALPLAPPEQDRSRNPIKIAPGKSGLAFKTCTIEGEGEKIYLGRVGTPVIYSYTNIGKEIVGIISNGQDVQTVERGYYCATFKCYGPGMSPMVTSTLTYVQAIPFLGIYYISGESWIPSLPPIEE

SEQ ID NO: 3 - DNA, 1140 bp, Zea mays
atgatgatgagtaaacaaatgctcgtcttgtccctgctcctagtgttgttcgagcttggatcgctgccgacgacatcgtgcaaaaaggtctttttcaacttatgggtgacaaaccagccagctaatgccacccaagatgcggggtgtgctaagaaagatgatgcgctctcctccgccgacaccattaaggtatggaattacatcgaccctgcctctcaattgagacctgaagatggcggttacacgaccattagcgagtccatcgccaacatccctgaggacaacgccaaacgctacctccttatcctcaaacctggtgttgtgttccgcgagaagctgttactcggtagaagcaagcctttcatcaccataatgtccgaggaccccatgaaccctgctgttatcgtctggaatgacactgccaccaccatgggcaaggacggcaagccccttggtgtggatggaagcagcaccatggccatagagtccgactattttgtcgcctacaacgttgtcttcaagaacgatgcaccgctaccaaagctaggggaaaagaaaggtgaggcactagcactgcgagtgatgggaacaaaggcaaccttctacaattgcaccatcgaaggcggccagggtgctctgtacgaccagacgggtctgcactacttcaaggcttgtgccatcaagggaaccatcgacttcatcttcggatctgccaagtcattttatgaggaatgcaaaatcgtttcggtgttgaaggaggcattggcattgccattggcaccaccggagcaggaccgctctagaaatcccatcaaaatcgccccaggaaagagcgggttggcattcaagacttgcacaatcgagggggaaggagaaaaaatttacttgggtagggtgggcacgcctgtgatctactcctacaccaatataggtaaggagattgtaggcataatatctaatggtcaggacgtccagactgtcgaaagggggtactactgcgccactttcaagtgttacgggcctgggatgtctccaatggtaacctcaactctgacctatgtccaggcaataccctttctcgggatatactacatctcgggggagtcgtggatcccgtccctaccacccattgaagaataa

SEQ ID NO: 4 - DNA, 1071 bp, Zea mays
acgacatcgtgcaaaaaggtctttttcaacttatgggtgacaaaccagccagctaatgccacccaagatgcggggtgtgctaagaaagatgatgcgctctcctccgccgacaccattaaggtatggaattacatcgaccctgcctctcaattgagacctgaagatggcggttacacgaccattagcgagtccatcgccaacatccctgaggacaacgccaaacgctacctccttatcctcaaacctggtgttgtgttccgcgagaagctgttactcggtagaagcaagcctttcatcaccataatgtccgaggaccccatgaaccctgctgttatcgtctggaatgacactgccaccaccatgggcaaggacggcaagccccttggtgtggatggaagcagcaccatggccatagagtccgactattttgtcgcctacaacgttgtcttcaagaacgatgcaccgctaccaaagctaggggaaaagaaaggtgaggcactagcactgcgagtgatgggaacaaaggcaaccttctacaattgcaccatcgaaggcggccagggtgctctgtacgaccagacgggtctgcactacttcaaggcttgtgccatcaagggaaccatcgacttcatcttcggatctgccaagtcattttatgaggaatgcaaaatcgtttcggtgttgaaggaggcattggcattgccattggcaccaccggagcaggaccgctctagaaatcccatcaaaatcgccccaggaaagagcgggttggcattcaagacttgcacaatcgagggggaaggagaaaaaatttacttgggtagggtgggcacgcctgtgatctactcctacaccaatataggtaaggagattgtaggcataatatctaatggtcaggacgtccagactgtcgaaagggggtactactgcgccactttcaagtgttacgggcctgggatgtctccaatggtaacctcaactctgacctatgtccaggcaataccctttctcgggatatactacatctcgggggagtcgtggatcccgtccctaccacccattgaagaataa

SEQ ID NO: 5 - protein, 398 aa, Zea mays
MVGGVRRCGLGLAMAVALLLAALVVVASGGAETRQKLPAGSGNDDDHAAVLSRLSNVIDPPGSWPPRADAVVAKRCGGVAAPPPCYTSIQAALKAASAPQEAEEVEDKYVVHVLAGVYDETVNITRRNVMLIGDGVGATVITGNKSNATGVHMDMTATVNALGHGFIAQNLTIRNTAGPDGRQAVALRSNSNKSVVYCCSIEGHEDTLYVENGIQFYLQTSIWGTVDFVFGNAQAMFQSCALLVRRPPKGKHNVLTAQGCNNASRESGFSFHMCTVEAAPGVDLDGVETYLGRPYRNFSHVAFIKSYLSRVVSPNGWVAWNKNKVVDDTTRTILYLEYGNDGAGADTAGRVKWPGFRVLNTDDEAIAYTADTFINASKWVPEPIQYVHTLGTAPPPRA

SEQ ID NO: 6 - protein, 369 aa, Zea mays
GAETRQKLPAGSGNDDDHAAVLSRLSNVIDPPGSWPPRADAVVAKRCGGVAAPPPCYTSIQAALKAASAPQEAEEVEDKYVVHVLAGVYDETVNITRRNVMLIGDGVGATVITGNKSNATGVHMDMTATVNALGHGFIAQNLTIRNTAGPDGRQAVALRSNSNKSVVYCCSIEGHEDTLYVENGIQFYLQTSIWGTVDFVFGNAQAMFQSCALLVRRPPKGKHNVLTAQGCNNASRESGFSFHMCTVEAAPGVDLDGVETYLGRPYRNFSHVAFIKSYLSRVVSPNGWVAWNKNKVVDDTTRTILYLEYGNDGAGADTAGRVKWPGFRVLNTDDEAIAYTADTFINASKWVPEPIQYVHTLGTAPPPRA

SEQ ID NO: 7 - DNA, 1197 bp, Zea mays
atggtaggcggcgtgaggaggtgcggcctgggcctggccatggcggtggccctgctgctcgctgcgctggttgtcgttgcaagcggcggcgcggagacgaggcagaagctgcctgctggcagtggtaacgacgacgaccacgcagccgttctcagccgcctgtccaacgtcattgatccgccggggagctggcctccgcgtgcggatgctgtcgtggcgaagcggtgccgcggcgtcgctgctccgccgccttgctacaccagcatccaggcagccgtggatcacgcaccagcaccacaagaagccgaggaggtggaggacaagtacgtcgtgcatgtgctcgctggcgtctacgacgagaccgtcaacataacaagaaggaacgtgatgctgatcggcgatggggtcggtgccaccgtcatcacggggaacaagagtaatgcaacaggcgtccacatggacatgacggcgacagtgaatgccttgggtcacggcttcatagcacagaacctgacaatcagaaacacggcagggccggaaggcaggcaggccgtggcgctaaggtcaaattcgaacaagtcggtcgtctactggtgcagcattgaaggtcatgaggacaccttgtacgtggagaacgggatccagttctacctgcagacctcgatctggggcaccgtggactttgtgtttggcaatgcccaggccatgttccagagctgcgcgctgctggtgcgccgcccaccgaaaggcaagcacaatgtgctgacggcccagggctgcaacaacgcaagccgcgagtccggcttctcgttccacatgtgcaccgtggaagccgcgccgggcgtggacctcgacggcgtggagacctacctcggccgcccctacaggaacttctcccacgtcgccttcatcaagtcgtatctcagtcgcgtggtcagccccaacggctgggtcgcgtggaacaagaacaaggtcgtcgaggataccacccggaccatcttatacctggaatacggcaacgacggsgccggtgccgacacagccggccgygtcaagtggccgggcttccgcgtcctcaacaccgacgacgaggcgatcgcgtacacggcggacacgttcatcaccgcgagcaagtgggtccctgagcctatccagtacgtccacaccctcggcacggcgccgccgccgcgcgcctga

SEQ ID NO: 8 - DNA, 1110 bp, Zea Mays
ggcgcggagacgaggcagaagctgcctgctggcagtggtaacgacgacgaccacgcagccgttctcagccgcctgtccaacgtcattgatccgccggggagctggcctccgcgtgcggatgctgtcgtggcgaagcggtgccgcggcgtcgctgctccgccgccttgctacaccagcatccaggcagccgtggatcacgcaccagcaccacaagaagccgaggaggtggaggacaagtacgtcgtgcatgtgctcgctggcgtctacgacgagaccgtcaacataacaagaaggaacgtgatgctgatcggcgatggggtcggtgccaccgtcatcacggggaacaagagtaatgcaacaggcgtccacatggacatgacggcgacagtgaatgccttgggtcacggcttcatagcacagaacctgacaatcagaaacacggcagggccggaaggcaggcaggccgtggcgctaaggtcaaattcgaacaagtcggtcgtctactggtgcagcattgaaggtcatgaggacaccttgtacgtggagaacgggatccagttctacctgcagacctcgatctggggcaccgtggactttgtgtttggcaatgcccaggccatgttccagagctgcgcgctgctggtgcgccgcccaccgaaaggcaagcacaatgtgctgacggcccagggctgcaacaacgcaagccgcgagtccggcttctcgttccacatgtgcaccgtggaagccgcgccgggcgtggacctcgacggcgtggagacctacctcggccgcccctacaggaacttctcccacgtcgccttcatcaagtcgtatctcagtcgcgtggtcagccccaacggctgggtcgcgtggaacaagaacaaggtcgtcgaggataccacccggaccatcttatacctggaatacggcaacgacggsgccggtgccgacacagccggccgygtcaagtggccgggcttccgcgtcctcaacaccgacgacgaggcgatcgcgtacacggcggacacgttcatcaccgcgagcaagtgggtccctgagcctatccagtacgtccacaccctcggcacggcgccgccgccgcgcgcctga

SEQ ID NO: 9 - DNA, 1661 bp, Zea mays
ataaatccatttttaaaattaaaataattccggaaaaggctgaaatcgtttttaatgtccaaaaaatatctcaaaggctccgaaaattctaggaaaaaaattcccaggcatgatttggcacacactgaactcaaaaaacatatttagaacttttgaaaatgttggaactacctcaaatgaatagattttggttctcggagagtagaaaaatattcagaaaaatatgaaaaattttcggaaacttatacaagatatattaacatgtttcaaacacttcttgcacttagaaacactatgcaacaacatgatgcaacatccaaagaacttctagggctcacgccaacatagaacaaaataactctccattattttagaaaggggaaaagtagagaaaggagagagtaacaactgaattttgagtatagagcaaagaaatttttgtattccaaaattcagggtgttacacggggcagcaagccggtggcggcgcgtcggctgtagagtaggggggcagagcgttattggcggtgtcgtaaagctagtgcatagaaaatgggcgagcgtgagcgcgtgtggatgaaaagttgggcacgatcctcataatgcattaaacctgtcgaccaagatcaagggccgacaaaagtgatcaatccccgcatgtgtgtgagatagtttgccaggcccacactaatccctgtcatctaggcttcatggctgacatgagttatttattctcatgagtaaggaggccaacgagagttatttaatcttcatcggctgacctcttggccgacagtaattaacttaacttttgccagcgtctttacgactgacgtttattagtttgtctctgtcgacattttaaaaggccgacatggattattttgcccgacgagagttaccctgattcctgttatgtgtgtataattaaagctacacgtactttgtaaaaaacaaaaatgaatagtagtttaggagtgcgggggtataatgcatggaccgtattagtagctttgtaacatactcctatttcaggatataggacagctagctagagcaatagcagagctaggtttacgggtaaccttgctggaaatgtggagcagtatgttcgcttaattttcatagcacatgttctgaatctttctacaaaaaaattcctatactaaatcgagttggagaatatatatttgcaaaacccacccacccaaccaaattacccccacatccataacttccactaaatccaccaaacaaattacacccaaacgtaacacaccaataaaaccaacgattcatattggtttgaggtcttgtacttatcgagtatggtttctaacagcttcagtaggatttatatgaaaagttttagtaaattcattttgttgaggaatacagactcgttaattttccatgcattaatcaaaggaacgtacatgcattcatcagaattcaacttctcaaattaggttgttggactcagctaatacctaacagccttccatcaacaactaaaacaaaggaacaccactccttgccatgttcaatcacacacctctcattctcgatagtcccaggctaggtatctataaataatgaacaatgaggaagggaggaataagtagattgagaaatactaagaagaagataacatctcgacc

SEQ ID NO: 10 - DNA, 6985 bp, Zea mays
cggtgcaggccagcccgcccatgcatccgacgatccagtgctcgaccgagatgcagcagcaagatatggttgggtatagctagagcgcagttggccggccagaacaatgctaggcgagagagggaagtggtggtgataggaatgtgagaggagagggattgttatctgttaggtcttatgaatatggaggaccggccggcagcaaacaaaaaagtgaacactcatcatagctgctgttgggcagggattcttttccatctcgcgtcctctggttgagagatatatatatgtctgctgctctcgctatatagagggccggcctgccctgctagctagctagctagctagttcaagttctaggcacggatcgatcaacattaatttaattacttaattattagttcccgacgaaattaatatatgttaaacaaagactttaatttttattatatccttatatatatatatatatatatatatattatatagttattatcagttttaatttaatccagaaaaccagtatgatgtatgtgatgacatgtatatgtgcatgtgtgtattactactcgcataagcagtaaacaacagtaaaacatgcacaatcactaagaacataatgtaccctgcggaggtagcgatactcaagacatcgttggctctattcacacgagacatttcgtgtgtatgttcgatttagtcgtatgcagtcgattctggtatatgtacgcagtgcggtccctcgaacggcatcgacgacgaagaaccggatcatcgttggtcgagaggatggagcgagcagttgcgagtacgctccccaaaaacctgatcgcccgcacacccgtgcaagtgtagctctacggacgtcgatttcggaggcctactctcccactctctctgtgctcgcggacggtgggacggtaacgggctgtgtcactacacgacggttgatctttagcgacccttattaggtaccaattgttggttgctaaaggttagggaccgacggtctgtcgctaaatccttttgtagcaacggtcggttggtcgctaaaagtttagaaatttaacgacttatagttggtcgctatagatgtcgtcggggattagagtccgtgacccagcgtctcaggacccaggctcccagtcccagcctcttctatccggcatccaaggagcagcaaatccagagacctcgactcctcctaaccctagccgccaggctccagtgctccaccgctccagcagatcccgaaagcagcgaggaaacacagggcgaccgaggaatgtctggactctggatccgccggtccgccacaccacccctcatctccgagtctccgacctgagcgcgccgccccgccccgccccgcctctccctcgggcgctgtccgcgcctccgtgcctctgcggtccgcgttcgggcgtccgcccagggcctaggcagttgtttcctgctcgcttgctgccgtccgccaggccgcctaggcagacatcacactacttctacagttctacgaccagcgcccagcggtccagtccaggttagtgcttttgccatcatttggtccaaatctctcttttcgttatttttacgtaccctagtgttgttccacaacgaaatgccaaaataattcttgttgatttttctattattggtgttagttcaattccgctacacattcaataaaaatgccaagctttttgtgacatatgcagaagaaccttatttacaaactgcttgatgaacattgttcattttgatgattagataaattttgtttttcagtatatctgtactcattctattgtaaacaattcattctagatatgtaacaaaatggcatgtttatatctttgattcatgatgcatttctttcatacaccatgctgtaagagtagaattgatgattctttcatcgcattcaataatctcacaagattgtcttccattttcatagttggtaccagtagtgactgagctcattgttgcccagttgatgtaccttgaatggatgaacagtaaaaaacctgtttatatatacatcaactcaactggaatggctcgcgatgatcgtgaaccggtaagcaagattattcaattggtgacttcagtcaagtatttccagtattggggtcagtatcggtagaatttggcttattcatgttcttctaattagggtagattataaggtctccttagcatttgtatcatgtcgtcatggttgttatatagtcaaatagttctttaattgattgtctgtatttgtcaggtcgggatggagagcgaaggttttgcaatctatgatgcaatgatgagtatgaaaactgaggtgctatgccttacatttactatctgcagttcaatagtgtaatgttgatcatttggtacagattgatgctgattatttactgaaacatgaacatctattttatgcaaaaaagttctcagtggacataatttatttttaaaagaagaattttactgtgtatgcaatctcgatctgcctagttctgtagagattactaatgattcaatgaatggcttgtcgatcatttgtttgaactggatggagcgtgtaaaaaacatgatcagcattggaggaactaggtggcaatggcatcataaccaagaagaaaattaggcttccaaatttgagatgaagacgatgactcaaacttgagccatggaggtgttcaccagcagccccaccaacttagttacaccgagttccgatggagtgtgtaaaagcaaacttctttgtgtatttatttgttcaatgaaagagtggccataaatacaaactttgcactgtatgctgatattctgaagtgatcagcaccctgttttcatcaccataagattaagatgggatgtttgtatcaaataaatctacagtttagccaaactttgcactagcgctgatatcaataacttgttaggtccctgtttcatcaacataacattaggaccggatgtttgtacaaattgaattcagtgtccaaactgtgaattgttaaaattcatcattttttctaaatttatggacatcatgatgaggttactatttgccatgcagatccacacactttgtataggagctgcagcaggccatgcatgtcttgtgcttgcagctggaaaaaaaggcaaacattatatgttccctcatgccaaaggtagtaacgtgataacttccgcacctgttttctttcccacgggaaatactcacttttgctcatcatttcctgctgattctggatcatactatgtcagctatgattcagcaaccacgtattccttcttatgggatgatgcaagcatcagatgttgttattcgtgcaaaggaggtacgggccatggttttttgagttgcttatattttgagtgttatttcgtattctaatgtatgcttatatttttatttgcaaatgtagcaacaatatactattttgttgtactttttctagggtgttctagacaaagaaagcttgtaccttaaaacaggaggatacaagaagctggagtggttgtctgaaggcttttgaactgcttgaagtattagtgtagctttggtgcttttgtgactgatcagctctggagcagctagagtgatgtagttgtgggcttttgaactgcttcaagctgaagtgatgtcgctgtggtgcttttgtgactgatcatctctagagcttttgtgaatgatctagctagctatctgtaaatgatctagttgtgaatgatattataatggtgatgcttttgtgaatatataattgtggtgcttttgtgtttatgtgatggatctatgactatggtatactgattaactgtgtatgtctttatgatttgtgtataaaattctgtgatttgtgatgcttaattaaatgtatgttattattaataacaacagtaaaaagggcgactttctgttggttgctaaatgtatagtatgccgacttacggttggttgctaaatctatagtacagcaacccacggttagtcgctaaatatacaatattagcaacggtcggtcggtcgctaaaaagatgtcggtcgctaaagcctttagcgacggcacttacaacgaccatccttatgggtcgctaaaagtttttagcgaccaaccgtaggtcgctgaaggccgttttagcaaccaaccgtaggtcactgtaagtgaaccgtcgtgtagtgtgtgcaacgcgtatgagagactgttcgcataactggaagagcgtccacctcctccgtatttatacacgcgcattgggaggggaacagactataacagtcgccatcagagtcagaaactgacagccattacagccagccagaaaccgatgtaccataagagacgcctgttactagccataagacaggaaacaactgatcattactccaggcaaaatgcgcaatcgttagaaagaaatatttggactaaggtccgatttaccacgagccacggcccggctcggccggcggcgcctgcgcacgattgggcagtcctcaatccttttctcaacttctcaacagatacaccaatggtccacctatctaagttaattgatttgtcctttgaacttccggtatggtactaaaatattagtgcaccatagcattaaagtggacctttaacattgactattattgaatattaatttgggccaagcccacattaatccaacaatccccatcaagtgctaagtcacactaaaaatgctcttatcatctcaaacgtttgatatacatgtgtttcgatggagactgttaagttgaacatccacctagaacctagactacacttaaccacaactgcacctagaactaatgaagatatttcagtaataaccaattgggtgaaactatatatatatatatatatatatatatatatatatatatataccatcgctatttgtcagtctacccaaccgataattattgagcggacttcagtaataatcgagaactaatgaacatcgttcagtaataaccaattaggctcctcatccacctctttcgtaaccaatcatcgaaattttagcacaattttccttgtgtagaactccatcagacggtctatctttccacattcattctcaacaaattttcctagcagcctcatccttcttgtgcctttgttaatacaacctaaaggccggtataaaataaccaaacatgacaaatacataaaacaaataaatcacaagaataaatttctccatgacatctgcattcaatctgctagctgaacattctatcgtgattagtgggtttgctaaagcaccaggataggcagtagaacgaagagcctctaaaaggaaagtttcagggtcacttgatgatttcgcaagctctgcctttagccttaaaatctaaaaagaaaacatgacactatgtcagcaaagaacatgatcttttaatctaaaacgacatgtaggggatgacatcaagatcaaatacctattcattgagctcccaattaagagagcagggttgcgtacacaatcaataagcacctcaacctggaatcccaggcatataagtcttttgggctccacaagtgtagctcatcatctcacgacaagtagatgctttggaacaaccacctattgcctcaattttacgcacaccacacaattcgctcttgttggtagtgaatgcaaagtccattgtatcttcaatagctgaccggcacctgctcagaaccataatcaacataaactccaacaaaatcagatggtccctgaatagaaaatatgaacaccctattatcatcagctactgctgagggattggatggaatgtgtaaccatataaaaataaagacaacatactgcagatgtctcagaggctattttagaacatacaatgttgagttgctgctctccactaatatatgttgtattaacactgaggagggtgcaacaaatgctcataaaagtaatatcaaactgtgaattggagttactattatgaaagacatttgttaggcaccaccacacgacaatgtatgcacatgtctctttaaaacagaaagaatagaagtgtaagaaataacgtatagacaatttatttggtaaaaaaaaggccagaagagtaggatggatcacgacgggcatccaagttgtttcatctctctgtgaagaaccttagatgtattgcaccccttgcagcagttctaaaaagttatttaaccatatgataaaagtagaaatatctacggttggatggacaacatccaagaatatataaagtaataaaaatttgcattttgaattttaaaattttgaatgtaatttttgagagcagagatgatttcaaataaaaaagttatcaactaaaaggtttcataacttttcgagatccacaacttttatgttggtggttttatcatccaaaatcgtttggaaaactcaaaaaattaaggatataaatgatttctagtggcggttttttaagaaaaccgccactagaaattatggtggttttcttaaggaactaccactaaaaaatagcactaacgatcagtctttaaaaaaaatacaccccactatgcaggctttggtgtaaagcaatcaccgtgcatcgtcgtcctcaacacactcggaaaaactatatatatagaatccataaataagagaggaaagaacccgtgcagaaatctttttagtttactgaatctttctgggaaactttttctttattaaatcgtcttgggggaacattttcctttactaaactactaggaggctgacatgaagggcccttcttccctgatccattattcttgccccgcgccggcttctcgtcatgggcacaccgcacatgcaggcttctggctcgatcgggcagggagggccgggcctatatatatgggatgctgccggcggtgttagccagcgaagacgcgcacaaaggtaagtgtgtaaggttaggagccagtgtaccatcacatcagatcagatcgatcgcagcttatagggg

SEQ ID NO: 11 - protein, 540 aa, Zea mays
GSGDGGRAEEGEMSTSVKSIKSFCQPVDYRETCEKALEAAAGNATSPTELAKAIFKVTSDRIAKAVRESALLNELKHDRRTSGALHNCGELLDYAIDDLRTTFDRLGGFEMTNFKSAVDDLRTWLSSALTYQETCLDGFENTTTPAAGKMRKALNSSQELTENILALVDEFSETLANLGLPSFHRRLLAEHARGAPSWMPDAKRRLLLVSPGEKGFRPDVTVAKDGSGDFRTINAALAKVPLKSATTYVMYVKAGKYREYVSVARNVTNLVMVGDGATKTVITGHKSFMMNITTKDTATMEAIGNGFLMRGIGVKNTAGAKNHQAVALRVQSDMSAFYECRFDGYQDTLYTHTSRQYYRDCVITGTIDFIFGNAQVVFQNCLIQVRKCMDNQQNIVTAQGRKERRSVGGTVIHNCTVAPHPEFEKSVGRFRTFLGRPWKEHSRTLYIQSEIGGFIDPQGWLPWLGDFGLSTCYYAEVENHGPGANMTRRVKWRGIKNITYQHALQKYTVESFIQGQHWLPQLGVPFIPGLLPQQQSGRIH

SEQ ID NO: 12 - DNA, 399 bp, Zea mays
taaagcaatcaccgtgcatcgtcgtcctcaacacactcggagaaactatatatatagaatccataaataagagaggaaagaacccgcgcagaaatctttttagtttactgaatctttctgggaaactttttctttattaaatcgttttgggggaacattttcctttactaaactactaggaggctgacatgaagggcccttcttccctgatccattattcttgccccgcgccggcttctcgtcatgggcacacggcacatgcaggcttctggctcgaccgggcctatatatatggatgctgccggtgttagccagcgaagacgcgcacaaaggtaagtgtgtaaggttaggagccagtgtaccatcacatcagatcagatcgatcgcagcttatagggg

SEQ ID NO: 13 - protein, 378 aa, Zea mays
MMMSKQMLVLSLLLVLFELGSLPTTSCKKVFFNLWVTNQPANATQDAGCAKKDDALSSADTIKVWNYIDPASQLRPEDGGYTTISESIANIPEDNAKRYLLILKPGWFREKLLLGRSKPFITIMSEDPMNPAVIVWNDTATTMGKDGKPLGVDGSSTMAIESDYFVAYNVVFKNDAPLPKLGEKKGEAPALRVMGTKATFYNCTIEGGQGALYDQTGLHYFKACAIKGTIDFIFGSAKSFYEECKIVSVLKEALVLPLAPPEQDRSRNPIEIAPGKSGLAFKTCTIEGEGEKIYLGRVGTPVIYSYTNIGKEIVGIISDGRDVQTVERGYYCATFRCYGPGMSPMVTSTLTYVEAIPFLGIHYISGESWIPSLPPAEE

SEQ ID NO: 14 - protein, 355 aa, Zea mays
TTSCKKVFFNLWVTNQPANATQDAGCAKKDDALSSADTIKVWNYIDPASQLRPEDGGYTTISESIANIPEDNAKRYLLILKPGWFREKLLLGRSKPFITIMSEDPMNPAVIVWNDTATTMGKDGKPLGVDGSSTMAIESDYFVAYNVVFKNDAPLPKLGEKKGEAPALRVMGTKATFYNCTIEGGQGALYDQTGLHYFKACAIKGTIDFIFGSAKSFYEECKIVSVLKEALVLPLAPPEQDRSRNPIEIAPGKSGLAFKTCTIEGEGEKIYLGRVGTPVIYSYTNIGKEIVGIISDGRDVQTVERGYYCATFRCYGPGMSPMVTSTLTYVEAIPFLGIHYISGESWIPSLPPAEE

SEQ ID NO: 15 - DNA, 1140 bp, Zea mays
atgatgatgagtaaacaaatgctcgtcttgtccctgctcctagtgttgttcgagcttggatctctgccgacgacatcgtgcaaaaaggtctttttcaacttatgggtgacaaaccagccagctaatgccacccaagatgcggggtgtgctaagaaagatgatgcgctctcctccgccgacaccattaaggtatggaattacatcgaccctgcctctcaattgagacctgaagatggcggttacacgaccattagcgagtccatcgccaacatccctgaggacaacgccaaacgctacctccttatcctcaaacctggtgttgtgttccgcgagaagctgttactcggtagaagcaagcctttcatcaccataatgtccgaggaccccatgaaccctgctgttatcgtctggaatgacactgccaccaccatgggcaaggacggcaagccccttggtgtggatggaagcagtaccatggccatagagtccgactattttgtcgcctacaacgttgtcttcaagaacgacgcgccgctaccaaagctaggggaaaagaaaggtgaggcaccagcactgcgagtgatgggaacaaaggcaaccttctacaattgcaccatcgaaggcggccagggtgctctgtacgaccagacgggtctgcactacttcaaggcttgtgccatcaagggaaccatcgacttcatcttcggatctgccaagtcattttatgaggaatgcaaaatcgtttcggtgttgaaggaggcattggtattgccattggcaccaccggagcaggaccgctctagaaatcccatcgaaatcgccccaggcaagagcgggttggcattcaagacttgcacaatcgagggggaaggagaaaaaatttacttgggtagggtgggcacgcctgtgatatactcctacactaatataggtaaggagattgtaggcataatatctgatggtcgggacgtccagacagtcgaaagggggtactactgcgccacttttaggtgttacgggcctgggatgtctccaatggtaacctcaactctgacctatgtcgaggcaataccctttctcgggatacactacatctcgggggagtcatggatcccgtccctaccacccgctgaagaataa

SEQ ID NO: 16 - DNA, 1075 bp, Zea mays
gccgacgacatcgtgcaaaaaggtctttttcaacttatgggtgacaaaccagccagctaatgccacccaagatgcggggtgtgctaagaaagatgatgcgctctcctccgccgacaccattaaggtatggaattacatcgaccctgcctctcaattgagacctgaagatggcggttacacgaccattagcgagtccatcgccaacatccctgaggacaacgccaaacgctacctccttatcctcaaacctggtgttgtgttccgcgagaagctgttactcggtagaagcaagcctttcatcaccataatgtccgaggaccccatgaaccctgctgttatcgtctggaatgacactgccaccaccatgggcaaggacggcaagccccttggtgtggatggaagcagtaccatggccatagagtccgactattttgtcgcctacaacgttgtcttcaagaacgacgcgccgctaccaaagctaggggaaaagaaaggtgaggcaccagcactgcgagtgatgggaacaaaggcaaccttctacaattgcaccatcgaaggcggccagggtgctctgtacgaccagacgggtctgcactacttcaaggcttgtgccatcaagggaaccatcgacttcatcttcggatctgccaagtcattttatgaggaatgcaaaatcgtttcggtgttgaaggaggcattggtattgccattggcaccaccggagcaggaccgctctagaaatcccatcgaaatcgccccaggcaagagcgggttggcattcaagacttgcacaatcgagggggaaggagaaaaaatttacttgggtagggtgggcacgcctgtgatatactcctacactaatataggtaaggagattgtaggcataatatctgatggtcgggacgtccagacagtcgaaagggggtactactgcgccacttttaggtgttacgggcctgggatgtctccaatggtaacctcaactctgacctatgtcgaggcaataccctttctcgggatacactacatctcgggggagtcatggatcccgtccctaccacccgctgaagaataa

SEQ ID NO: 17 - protein, 398 aa, Zea mays
MVGGVRRCGLGLAMAVALLLAALVVVASGGAEMRQKLPAGSGNDDDHAAVLSRLSNVIDPPGSWPPRADAVVAKRCRGVAAPPPCYTSIQAAVDHAPAPQEAEEVEDKYVVHVLAGVYDETVNITRRNVMLIGDGVGATVITGNKSNATGVHMDMTATVNALGHGFIAQNLTIRNTAGPEGRQAVALRSNSNKSVVYWCSIEGHEDTLYVENGIQFYLQTSIWGTVDFVFGNAQAMFQSCALLVRRPPKGKHNVLTAQGCNNASRESGFSFHMCTVEAAPGVDLDGVETYLGRPYRNFSHVAFIKSYLSRVVSPNGWVAWNKNKVVEDTTRTILYLEYGNDGAGADTAGRVKWPGFRVLNTDDEAIAYTADTFINASKWVPEPIQYVHTLGTAPPPRA

SEQ ID NO: 18 - protein, 369 aa, Zea mays
GAEMRQKLPAGSGNDDDHAAVLSRLSNVIDPPGSWPPRADAVVAKRCRGVAAPPPCYTSIQAAVDHAPAPQEAEEVEDKYVVHVLAGVYDETVNITRRNVMLIGDGVGATVITGNKSNATGVHMDMTATVNALGHGFIAQNLTIRNTAGPEGRQAVALRSNSNKSVVYWCSIEGHEDTLYVENGIQFYLQTSIWGTVDFVFGNAQAMFQSCALLVRRPPKGKHNVLTAQGCNNASRESGFSFHMCTVEAAPGVDLDGVETYLGRPYRNFSHVAFIKSYLSRVVSPNGWVAWNKNKVVEDTTRTILYLEYGNDGAGADTAGRVKWPGFRVLNTDDEAIAYTADTFINASKWVPEPIQYVHTLGTAPPPRA

SEQ ID NO: 19 - DNA, 1594 bp, Zea mays
atggtaggcggcgtgaggaggtgcggcctgggcctggccatggcggtggccctgctgctcgctgcgctggttgtcgttgcaagcggcggcgcggagatgaggcagaagctgcctgctggcagtggtaacgacgacgaccacgcagccgttctcagccgcctgtccaacgtcattgatccgccggggagctggcctccgcgtgcggatgctgtcgtggcgaagcggtgcggcggcgtcgctgctccgccgccttgctacaccagcatccaggcagccctgaaagccgcatcagcaccacaagaagccgaggaggtggaggacaagtacgtcgtgcatgtgcttgctggcgtctacgacgagaccgtcaacataacaagaaggaacgtgatgctgatcggcgatggggtcggtgccaccgtcatcacggggaacaagagcaatgcaacaggcgtccacatggacatgacggcgacagtgagtgagtaactctggcgatcgatatttcactgcagttgagctgtcatgcatgcatactgtaaatcagaacagaactattaatttgctgctgctgctgcagatgccttgggtcacggcttcatagcacagaacctgacaatcagaaacacggcagggccggacggcaggcaggccgtggcgctaaggtcaaattcgaacaagtcggtcgtctactgttgcagcattgaaggtcatgaggacaccttgtacgtggagaacgggatccagttctacctgcagacctcgatctggggcaccgtggactttgtgtttggcaatgcccaggccatgttccagagctgcgcgctgctggtgcgccgcccaccgaaaggcaagcacaatgtgctgacggcccagggctgcaacaacgcaagccgcgagtccggcttctcgttccacatgtgcaccgtggaagccgcgccgggcgtggacctcgacggcgtggagacctacctcggccgcccctacaggaacttctcccacgtcgccttcatcaagtcgtatctcagtcgcgtggtcagccccaacggctgggtcgcgtggaacaagaacaaggtcgtcgacgataccacccggaccatcttatacctggaatacggcaacgacggcgccggtgccgacacagccggccgcgtcaagtggccgggcttccgcgtcctcaacaccgacgacgaggcgatcgcgtacacggcggacacgttcatcaacgcgagcaagtgggtccctgagcctatccagtacgtccacaccctcggcacggcgccgccgccgcgcgcctgacgatgttcggctcggcgcgcttaagttagtactgtactacgcaggctgctggcgtactgtactacggtattgtatgtctctagtatccagccatgcatcattgccgagatgatttaaggcatattgtggcatgcatgacgccgtatatttcgcgtatgcatgcatgtacctggacggctggacctctacctgtagctaccatttctctaccggtattgtacgtactctctttgtatgtgtgtctatattatatatgtatcctcgatctaaataaatggattacatctatcattatttc

SEQ ID NO: 20 - DNA, 1047 bp, Zea mays
gttctcagccgcctgtccaacgtcattgatccgccggggagctggcctccgcgtgcggatgctgtcgtggcgaagcggtgcggcggcgtcgctgctccgccgccttgctacaccagcatccaggcagccctgaaagccgcatcagcaccacaagaagccgaggaggtggaggacaagtacgtcgtgcatgtgcttgctggcgtctacgacgagaccgtcaacataacaagaaggaacgtgatgctgatcggcgatggggtcggtgccaccgtcatcacggggaacaagagcaatgcaacaggcgtccacatggacatgacggcgacagtgaatgccttgggtcacggcttcatagcacagaacctgacaatcagaaacacggcagggccggacggcaggcaggccgtggcgctaaggtcaaattcgaacaagtcggtcgtctactgttgcagcattgaaggtcatgaggacaccttgtacgtggagaacgggatccagttctacctgcagacctcgatctggggcaccgtggactttgtgtttggcaatgcccaggccatgttccagagctgcgcgctgctggtgcgccgcccaccgaaaggcaagcacaatgtgctgacggcccagggctgcaacaacgcaagccgcgagtccggcttctcgttccacatgtgcaccgtggaagccgcgccgggcgtggacctcgacggcgtggagacctacctcggccgcccctacaggaacttctcccacgtcgccttcatcaagtcgtatctcagtcgcgtggtcagccccaacggctgggtcgcgtggaacaagaacaaggtcgtcgacgataccacccggaccatcttatacctggaatacggcaacgacggcgccggtgccgacacagccggccgcgtcaagtggccgggcttccgcgtcctcaacaccgacgacgaggcgatcgcgtacacggcggacacgttcatcaacgcgagcaagtgggtccctgagcctatccagtacgtccacaccctcggcacggcgccgccgccgcgcgcc

SEQ ID NO: 21 - protein, 631 aa, Zea mays
DDNDGNMLSSVKVSTVCAFTRYPEKCEQSLKHVVSDTSSPEDVFRDALNVALDEVSTAFQRSAHIGKDDATEDLRALARVKPADVVRHVKDLRVWVSGIMTYVYTCADGFEKPELKEAMDKVLQNSTELSSNALAILTRLGDLMPGKAKDLQATLAGAVGHDRRLLGWQIGDAEEVTSGGRGLLDEIVGVANANRKLLSDTLDEITGMSHGANGRRLLSSLGSRISSAQGDDVLARHQLLGVSPDDETDNAARRNLLSTELESIASTSAEANRQLLAAEELPDELAGKRELLSRTLMGIDEAATEAKRQLDEATTENTMSGDHRVLTTGLIGTFDEIQDGRSGVPPSDFPKWLPATQRRLLQQTQKPNTVVAQDGSGDFKTITEAITAVPNTFEGRFVIYVKAGTYKEYVTVPKNMANIFMYGDGPTQTVVTGDKSNAGGFATFASATFSAEGNGFICKSMGFVNTAGPEGHQAVAMHVQGDKSVFYNCRFEGYQDTLYVHANRQFFRDCEVLGTVDFIFGNSAALFQNCLMTVRKPGDSQSNMVTAQGRTDPNMPTGIVLQGCRIVPEQALFPDRLQIATYLGRPWKEYARTVVMESTIGDLIRPEGWAEWMGDLGLKTLYYAEYANTGP

SEQ ID NO: 22 - protein, 660 aa, Zea mays
MANRVTVASVIAAVGIVAVIGTMATVTSADDNDGNMLSSVKVSTVCAFTRYPEKCEQSLKHVVSDTSSPEDVFRDALNVALDEVSTAFQRSAHIGKDDATEDLRALARVKPADVVRHVKDLRVWVSGIMTYVYTCADGFEKPELKEAMDKVLQNSTELSSNALAILTRLGDLMPGKAKDLQATLAGAVGHDRRLLGWQIGDAEEVTSGGRGLLDEIVGVANANRKLLSDTLDEITGMSHGANGRRLLSSLGSRISSAQGDDVLARHQLLGVSPDDETDNAARRNLLSTELESIASTSAEANRQLLAAEELPDELAGKRELLSRTLMGIDEAATEAKRQLDEATTENTMSGDHRVLTTGLIGTFDEIQDGRSGVPPSDFPKWLPATQRRLLQQTQKPNTVVAQDGSGDFKTITEAITAVPNTFEGRFVIYVKAGTYKEYVTVPKNMANIFMYGDGPTQTVVTGDKSNAGGFATFASATFSAEGNGFICKSMGFVNTAGPEGHQAVAMHVQGDKSVFYNCRFEGYQDTLYVHANRQFFRDCEVLGTVDFIFGNSAALFQNCLMTVRKPGDSQSNMVTAQGRTDPNMPTGIVLQGCRIVPEQALFPDRLQIATYLGRPWKEYARTVVMESTIGDLIRPEGWAEWMGDLGLKTLYYAEYANTGP

SEQ ID NO: 23 - DNA, 1140 bp, Zea mays; identical to SEQ ID NO: 15

SEQ ID NO: 24 - DNA, 1071 bp, Zea mays
acgacatcgtgcaaaaaggtctttttcaacttatgggtgacaaaccagccagctaatgccacccaagatgcggggtgtgctaagaaagatgatgcgctctcctccgccgacaccattaaggtatggaattacatcgaccctgcctctcaattgagacctgaagatggcggttacacgaccattagcgagtccatcgccaacatccctgaggacaacgccaaacgctacctccttatcctcaaacctggtgttgtgttccgcgagaagctgttactcggtagaagcaagcctttcatcaccataatgtccgaggaccccatgaaccctgctgttatcgtctggaatgacactgccaccaccatgggcaaggacggcaagccccttggtgtggatggaagcagtaccatggccatagagtccgactattttgtcgcctacaacgttgtcttcaagaacgacgcgccgctaccaaagctaggggaaaagaaaggtgaggcaccagcactgcgagtgatgggaacaaaggcaaccttctacaattgcaccatcgaaggcggccagggtgctctgtacgaccagacgggtctgcactacttcaaggcttgtgccatcaagggaaccatcgacttcatcttcggatctgccaagtcattttatgaggaatgcaaaatcgtttcggtgttgaaggaggcattggtattgccattggcaccaccggagcaggaccgctctagaaatcccatcgaaatcgccccaggcaagagcgggttggcattcaagacttgcacaatcgagggggaaggagaaaaaatttacttgggtagggtgggcacgcctgtgatatactcctacactaatataggtaaggagattgtaggcataatatctgatggtcgggacgtccagacagtcgaaagggggtactactgcgccacttttaggtgttacgggcctgggatgtctccaatggtaacctcaactctgacctatgtcgaggcaataccctttctcgggatacactacatctcgggggagtcatggatcccgtccctaccacccgctgaagaataa

SEQ ID NO: 25 - protein, 404 aa, Zea mays
MESRRKNQQKTSYIVISIQLLAALLLLELVSLVPGASCEELPLDFWVSALRGVGAKDVSTADGCTKKNKDTVLCSAQANTVTNFINPTNSEEQGYRTIGESIANIPDDSTKRYILILSGGTVYREKVLVSKSKPFVTIRSDDPINPAIIVWNDTAATLGKDSKPLGVDGSSTMTVESDYFIAYGVVFRNDAAAAAKKKKAEGEAPALRVLGTKATFYNCTIEGGQGALYDQMGLHYFKSCTIRGTIDFIFGSAKSFYEDCTIVSVNNMEEIMTLPVAPPQLDIHDNPIKVAPGEGGFSFKTCTITGEGQQIFLGRMGTPSIYSYTQIAKEVVPIIYDKGNIFMPSNMTGRRCATFKCYGPGLEKIWHVKLRYAEAIYFLGTDFINGDSWILSIPPTDAETLLSV

SEQ ID NO: 26 - protein, 366 aa, Zea mays
EELPLDFWVSALRGVGAKDVSTADGCTKKNKDTVLCSAQANTVTNFINPTNSEEQGYRTIGESIANIPDDSTKRYILILSGGTVYREKVLVSKSKPFVTIRSDDPINPAIIVWNDTAATLGKDSKPLGVDGSSTMTVESDYFIAYGVVFRNDAAAAAKKKKAEGEAPALRVLGTKATFYNCTIEGGQGALYDQMGLHYFKSCTIRGTIDFIFGSAKSFYEDCTIVSVNNMEEIMTLPVAPPQLDIHDNPIKVAPGEGGFSFKTCTITGEGQQIFLGRMGTPSIYSYTQIAKEVVPIIYDKGNIFMPSNMTGRRCATFKCYGPGLEKIWHVKLRYAEAIYFLGTDFINGDSWILSIPPTDAETLLSV

SEQ ID NO: 27 - DNA, 1215 bp, Zea mays
atggagagcaggaggaagaatcaacagaagacgagctacattgtaatttccatccagctcctagcggccctcctactcttggagcttgtgtcgctagtgccgggggcgtcatgtgaggagttgcccttggatttttgggtgagtgcactacggggggtcggggcaaaggacgtttcaacagccgatggatgcaccaagaagaataaggacaccgtgctctgctctgcccaggctaacacggtaaccaatttcatcaaccctaccaactctgaggaacaaggctataggaccatcggggagtccattgctaacatccctgatgatagcaccaaacggtacatcctcatcctcagcggtggcaccgtgtaccgagagaaggtattggtgagcaaaagcaagccattcgtcaccataagatcagatgaccccatcaaccctgccatcattgtgtggaacgacactgccgccaccctggggaaggatagcaagccccttggagtagatggtagtagcaccatgaccgtagagtccgactacttcattgcttatggtgtcgtctttaggaatgatgctgcagcagcagcgaagaagaagaaggcagaaggcgaggcgccagcgttgcgggtgctaggaacaaaggcaaccttctacaactgcacaattgaaggtggacaaggcgccctatatgaccagatggggctgcactacttcaagtcctgcaccatcaggggaaccatcgacttcatctttggctctgccaagtccttttacgaggactgcaccattgtttccgtgaacaacatggaggagatcatgaccttgcctgtggcaccacctcaacttgacattcacgacaatccaatcaaagttgccccaggggagggcggcttctccttcaagacatgtaccatcactggggaagggcaacaaatcttcctcggaaggatgggcacgccttccatctactcctacacccagattgctaaggaggttgtgcccataatctacgacaaagggaacatcttcatgcccagtaatatgactggtagacgctgtgccactttcaagtgctatggacctgggttagagaaaatatggcacgtcaaacttagatacgctgaagccatatactttcttgggacagattttatcaacggagattcatggatcctgtccataccacctactgatgctgaaacattgctatcagtttga

SEQ ID NO: 28 - DNA, 1101 bp, Zea mays
gaggagttgcccttggatttttgggtgagtgcactacggggggtcggggcaaaggacgtttcaacagccgatggatgcaccaagaagaataaggacaccgtgctctgctctgcccaggctaacacggtaaccaatttcatcaaccctaccaactctgaggaacaaggctataggaccatcggggagtccattgctaacatccctgatgatagcaccaaacggtacatcctcatcctcagcggtggcaccgtgtaccgagagaaggtattggtgagcaaaagcaagccattcgtcaccataagatcagatgaccccatcaaccctgccatcattgtgtggaacgacactgccgccaccctggggaaggatagcaagccccttggagtagatggtagtagcaccatgaccgtagagtccgactacttcattgcttatggtgtcgtctttaggaatgatgctgcagcagcagcgaagaagaagaaggcagaaggcgaggcgccagcgttgcgggtgctaggaacaaaggcaaccttctacaactgcacaattgaaggtggacaaggcgccctatatgaccagatggggctgcactacttcaagtcctgcaccatcaggggaaccatcgacttcatctttggctctgccaagtccttttacgaggactgcaccattgtttccgtgaacaacatggaggagatcatgaccttgcctgtggcaccacctcaacttgacattcacgacaatccaatcaaagttgccccaggggagggcggcttctccttcaagacatgtaccatcactggggaagggcaacaaatcttcctcggaaggatgggcacgccttccatctactcctacacccagattgctaaggaggttgtgcccataatctacgacaaagggaacatcttcatgcccagtaatatgactggtagacgctgtgccactttcaagtgctatggacctgggttagagaaaatatggcacgtcaaacttagatacgctgaagccatatactttcttgggacagattttatcaacggagattcatggatcctgtccataccacctactgatgctgaaacattgctatcagtttga

SEQ ID NO: 29 - protein, 280 aa, Zea mays
MAAVGRSSLSLVLVMAAVIISGAGGERVPPAAKEPWPPAPHAVVVQQARLEECHRVAPACYTSIGQALAEAGKLVPKVKHRFVVLIKTGEYIEQVNITRRNVVLLGEGRGNTVISGNLSNLTGTAMVMTATVNVVADGFLAQNLTIRNEAGPKGRQAVALRSNSNRTVVFGCAIEGFEDSLYAENGVQVYLETDIYGTVDFIFGNAKAVFQRCRILVRRPIPGKHNVVTAQGCDNNMYENSGFVFHRCSVEADPNPWPVGQNLTGVETWLGGVGQGTRHQ

SEQ ID NO: 30 - protein, 255 aa, Zea mays
ERVPPAAKEPWPPAPHAVVVQQARLEECHRVAPACYTSIGQALAEAGKLVPKVKHRFVVLIKTGEYIEQVNITRRNVVLLGEGRGNTVISGNLSNLTGTAMVMTATVNVVADGFLAQNLTIRNEAGPKGRQAVALRSNSNRTVVFGCAIEGFEDSLYAENGVQVYLETDIYGTVDFIFGNAKAVFQRCRILVRRPIPGKHNVVTAQGCDNNMYENSGFVFHRCSVEADPNPWPVGQNLTGVETWLGGVGQGTRHQ

SEQ ID NO: 31 - DNA, 843 bp, Zea mays
atggcagcagttggcagatcgtcgctgtcgctcgtgctggtgatggcggcggtgatcatctccggcgccggtggcgagcgggtgcctcctgccgcaaaggagccctggccgccggctccgcatgctgtggtcgtacagcaggcgcggctggaggagtgccaccgagtggctccggcgtgctacaccagcatcggacaagctctggctgaagctggcaagcttgtccccaaggtgaagcaccgttttgttgtactcatcaagaccggagagtacatagaacaggtgaacatcacgagacggaacgtcgtcctgctcggcgagggcagaggcaacacagtaatttctggcaacctaagcaacctaacggggacggcgatggtgatgacggccaccgtgaatgttgtagccgacggctttttggcccagaacctgacgatccgtaacgaggccgggccgaaaggaaggcaggcggtggctttgaggtcaaactccaaccggaccgtcgtatttggctgtgctatagaaggcttcgaagatagcctgtacgccgaaaatggagtccaggtgtacctggagacggacatatacgggaccgtggattttatattcggaaacgcgaaggcggtcttccagcgttgccgcatcctggtgcggcggcccatccccggcaagcacaacgtggtgacggcgcagggctgcgacaacaatatgtacgagaactccggcttcgtcttccaccggtgcagcgtcgaagctgacccgaatccatggccggtgggccagaacctgaccggcgtggaaacatggctgggtggcgtgggacagggcacacgtcatcaatga

SEQ ID NO: 32 - DNA, 768 bp, Zea mays
gagcgggtgcctcctgccgcaaaggagccctggccgccggctccgcatgctgtggtcgtacagcaggcgcggctggaggagtgccaccgagtggctccggcgtgctacaccagcatcggacaagctctggctgaagctggcaagcttgtccccaaggtgaagcaccgttttgttgtactcatcaagaccggagagtacatagaacaggtgaacatcacgagacggaacgtcgtcctgctcggcgagggcagaggcaacacagtaatttctggcaacctaagcaacctaacggggacggcgatggtgatgacggccaccgtgaatgttgtagccgacggctttttggcccagaacctgacgatccgtaacgaggccgggccgaaaggaaggcaggcggtggctttgaggtcaaactccaaccggaccgtcgtatttggctgtgctatagaaggcttcgaagatagcctgtacgccgaaaatggagtccaggtgtacctggagacggacatatacgggaccgtggattttatattcggaaacgcgaaggcggtcttccagcgttgccgcatcctggtgcggcggcccatccccggcaagcacaacgtggtgacggcgcagggctgcgacaacaatatgtacgagaactccggcttcgtcttccaccggtgcagcgtcgaagctgacccgaatccatggccggtgggccagaacctgaccggcgtggaaacatggctgggtggcgtgggacagggcacacgtcatcaatga

SEQ ID NO: 33 - DNA, 2053 bp, Zea mays
gaataaaaccaattatttgttttatttttccatcgcttatatatatatgcaccagagatttttgtgttacccttgcatatatgcatgcatgcatgtggaatgaatccaagtctaaaattaggcgcctaatggtgaccttccatatattagacaccttaattatttgaattggattttggatgcaaactagcttattagctatctatatataacaagattaaagaagaagacagacaacctaattaaagaatttgaggagagaaagaaagaaagagtgatcgtcatggagagcaggaggaagaatcaacagaagacgagctacattgtaatttccatccagctcctagcggccctcctactcttggagcttgtgtcgctagtgccgggggcgtcatgtgaggagttgcccttggatttttgggtgagtgcactacggggggtcggggcaaaggacgtttcaacagccgatggatgcaccaagaagaataaggacaccgtgctctgctctgcccaggctaacacggtaaccaatttcatcaaccctaccaactctgaggaacaaggctataggaccatcggggagtccattgctaacatccctgatgatagcaccaaacggtacatcctcatcctcagcggtggcaccgtgtaccgagagaaggtattggtgagcaaaagcaagccattcgtcaccataagatcagatgaccccatcaaccctgccatcattgtgtggaacgacactgccgccaccctggggaaggatagcaagccccttggagtagatggtagtagcaccatgaccgtagagtccgactacttcattgcttatggtgtcgtctttaggaatgatgctgcagcagcagcgaagaagaagaaggcagaaggcgaggcgccagcgttgcgggtgctaggaacaaaggcaaccttctacaactgcacaattgaaggtggacaaggcgccctatatgaccagatggggctgcactacttcaagtcctgcaccatcaggggaaccatcgacttcatctttggctctgccaagtccttttacgaggactgcaccattgtttccgtgaacaacatggaggagatcatgaccttgcctgtggcaccacctcaacttgacattcacgacaatccaatcaaagttgccccaggggagggcggcttctccttcaagacatgtaccatcactggggaagggcaacaaatcttcctcggaaggatgggcacgccttccatctactcctacacccagattgctaaggaggttgtgcccataatctacgacaaagggaacatcttcatgcccagtaatatgactggtagacgctgtgccactttcaagtgctatggacctgggttagagaaaatatggcacgtcaaacttagatacgctgaagccatatactttcttgggacagattttatcaacggagattcatggatcctgtccataccacctactgatgctgaaacattgctatcagtttgaatatatagaatcctcagagatcatcaaagatcatcatctaccactaattaataatgagccaaattaaataagttttgcgtatatattttatccctatctataaattatgcatgtgttgtaaacaagtcgtggatgggcttcatttgttctcgcgattgaaaaataacaaataaacaaagtagaaaagaggatgaaccagaagtcttgttcagcaagaaggaaggaggaatgacgatatttcccatgtgatacttgtaaatcatatgtgtaaacttcgtgggcgtacttgtaatttcatacgaagttgtacctctcgcctataaatagagtaacagtgtcatgcataaattcacctttcgagagctcaaagcttcatttagctcagccttcgaagtacctttgagctatcacttgtatgaagccgaaggtatgtttgtaattgtttgttatattgcaaggagtgaaataataagtgtggaagcatgaagatgagtaacatgtcttgctcgtaactgtttacatttcatccttgttaatatataagcatttgtgccc

SEQ ID NO: 34 - DNA, 1260 bp, Zea mays
cgcggccagcctatatatatatggtatcggacctgaggctgctggagtgctagttgagcttgagcaaggttgagtgaagagagtgacacacgcacacgacgcactacgttggcccgctacgagctgcaactggatcatggcagcagttggcagatcgtcgctgtcgctcgtgctggtgatggcggcggtgatcatctccggcgccggtggcgagcgggtgcctcctgccgcaaaggagccctggccgccggctccgcatgctgtggtcgtacagcaggcgcggctggaggagtgccaccgagtggctccggcgtgctacaccagcatcggacaagctctggctgaagctggcaagcttgtccccaaggtgaagcaccgttttgttgtactcatcaagaccggagagtacatagaacaggtgaacatcacgagacggaacgtcgtcctgctcggcgagggcagaggcaacacagtaatttctggcaacctaagcaacctaacggggacggcgatggtgatgacggccaccgtgaatgttgtagccgacggctttttggcccagaacctgacgatccgtaacgaggccgggccgaaaggaaggcaggcggtggctttgaggtcaaactccaaccggaccgtcgtatttggctgtgctatagaaggcttcgaagatagcctgtacgccgaaaatggagtccaggtgtacctggagacggacatatacgggaccgtggattttatattcggaaacgcgaaggcggtcttccagcgttgccgcatcctggtgcggcggcccatccccggcaagcacaacgtggtgacggcgcagggctgcgacaacaatatgtacgagaactccggcttcgtcttccaccggtgcagcgtcgaagctgacccgaatccatggccggtgggccagaacctgaccggcgtggaaacatggctgggtggcgtgggacagggcacacgtcatcaatgataccacgaagagcgtgagatacatggagtatgccaacacaggtcccggcgccgacaccacccaccgcgtcgactgggagggcgtccatgttctccacgaccccgcccaggtagccaaatacacgatagatgccttcatatcgggtaaggagtggattccccatcagatcccgtatgaccatgaggtccccagcgggcgcggcgccatcatcgtcaactagccggccggcggccagtctccctcccatcatctgcgcgcttcaattcatctgctccctcctg

(19) United States
(12) Patent Application Publication
(10) Pub. No.: US 2021/0238622 A1
(43) Pub. Date: Aug. 5, 2021

(54) POLLINATION BARRIERS AND THEIR USE

(71) Applicant: Carnegie Institution of Washington, Washington, DC (US)

(72) Inventors: Matthew EVANS, Stanford, CA (US); Yongxian LU, Palo Alto, CA (US); Samuel HOKIN, Sante Fe, NM (US); Jerry KERMICLE, Madison, WI (US)

(21) Appl. No.: 17/119,133

(22) Filed: Dec. 11, 2020

Related U.S. Application Data
(60) Provisional application No. 62/948,193, filed on Dec. 13, 2019.

(51) Int. Cl. C12N 15/82

(52) U.S. Cl. CPC C12N 15/8247; C12N 15/8218

(57) ABSTRACT

The present disclosure relates to plant reproductive barriers such as those conferred by the Teosinte crossing barrier 1-female (Tcb1-female) gene, the Teosinte crossing barrier 1-male (Tcb1-male) gene, the gametophytic factor2 female (GA2-female) gene, and the gametophytic factor2 male (GA2-male) gene. The disclosure provides compositions that contain nucleic acid(s) comprising Tcb1-female, Tcb1-male, GA2-female, and/or GA2-male nucleic acid sequences, such as expression cassettes, vectors, transformed host cells, and genetically engineered plants and uses of these compositions that include for example, controlling plant pollination and overcoming species pollination barriers, conferring cross-incompatibility and self-incompatibility in plants, and isolating a breeding population of plants, such as a genetically modified plant population.

### CROSS REFERENCE TO RELATED APPLICATIONS

This application claims the benefit of U.S. Provisional Patent Application No. 62/948,193, filed Dec. 13, 2019, which is incorporated herein by reference in its entirety.

### GOVERNMENT INTEREST

The invention was made with government support under grant number IOS-0951259 awarded by the National Science Foundation and grant number 35301-13314 awarded by the United States Department of Agriculture-National Research Initiative Competitive Grants Program. The U.S. government has certain rights in the invention.

### REFERENCE TO SEQUENCE LISTING SUBMITTED ELECTRONICALLY

The content of the electronically submitted sequence listing (Name: (6146_0181_Sequence_Listing; Size: 89.3 kilobytes; and Date of Creation: Dec. 11, 2020) filed with the application is incorporated herein by reference in its entirety.

### TECHNICAL FIELD

The disclosure relates to the field of plant breeding and plant molecular biology.

### BACKGROUND

A reliable method of controlling fertility in plants offers the opportunity for improved plant breeding. This is especially true for development of maize hybrids.

Maize (Zea mays L.), also known as corn, has separate male and female flowers on the same plant, located on the tassel and the ear, respectively, and can be bred by both self-pollination and cross-pollination techniques. Most commercial maize is produced from hybrid seed produced by homozygous inbred maize lines. The production of hybrid maize seed requires the elimination or inactivation of pollen produced by the female parent. Incomplete removal or inactivation of this pollen results in undesirable self-pollinated non-hybrid seed that is unintentionally harvested and packaged with hybrid seed. Several methods have been developed in an attempt to control male fertility and thus prevent self-pollination. These methods include manual or mechanical emasculation (commonly referred to as detasseling, wherein the pollen-bearing tassels are removed from the plant which is to be used as the female, prior to pollen being shed), cytoplasmic male sterility, genetic male sterility, and chemical applications (e.g., gametocides, pollen suppressants, and chemical hybridizing agents). However, these alternatives are costly and unreliable and each of the alternatives has its drawbacks.

Thereupon, there is a need in the art for genetically and physiologically well-characterized cross-incompatibility systems in maize which prevent the indiscriminate hybridization of maize plants from unwanted pollen sources.

In domesticated maize, cross-incompatibility ranges in degree from creating a preference among pollen classes up to preventing seed set. Genes responsible for these effects are called gametophyte factors (hereinafter “GA”) because the efficiency of pollen function is affected (see, e.g., Nelson, O. E., The Maize Handbook, Freeling and Walbot, eds. Springer-Verlag (1993)). GAfactors conferring only a preference among pollen genotypes are cryptic, influencing the transmission of linked genes and the competitive ability of pollen in mixtures. Examples that involve recognition between corresponding alleles in pollen and silks are Ga2, Ga4, Ga8, and certain combinations involving Ga1. Incompatibility leading to failure of seed set occurs in conjunction with the strong allele of Ga1, specifically when Ga1-s Ga1-s plants are pollinated with ga1 ga1, the cross used to isolate commercial popcorn from the pollen of other maize plants. As a system of isolation, Ga1-s is imperfect because some maize strains carry a ga1-s or yet another allele, Ga1-m, which permits these strains to cross to strains containing both ga1 and Ga1-s. In these strains, the pollination barrier breaks down.

Accordingly, there is a need for improved mechanisms for controlling plant fertility reproductive barriers. The disclosure provides transgenic plants and other compositions and methods of making and using these compositions that address this need.

### BRIEF SUMMARY

The disclosure relates to plant reproductive barriers such as those conferred by the Teosinte crossing barrier 1-female (Tcb1-female) gene, the Teosinte crossing barrier 1-male (Tcb1-male) gene, the gametophytic factor2 female (GA2-female) gene, and the gametophytic factor2 male (GA2-male) gene. The disclosure provides compositions that contain nucleic acid(s) comprising Tcb1-female, Tcb1-male, GA2-female, and/or GA2-male nucleic acid sequences, such as expression cassettes, vectors, transformed host cells, and genetically engineered plants and uses of these compositions that include for example, controlling plant pollination and overcoming species pollination barriers, conferring cross-incompatibility and self-incompatibility in plants, and isolating a breeding population of plants, such as a genetically modified plant population.

In some embodiments, the disclosure provides compositions that contain nucleic acid(s) comprising Tcb1-female (Tcb1-f) coding and/or regulatory sequences, such as expression cassettes, vectors, transformed host cells, and genetically engineered plants. In some embodiments, the disclosure provides compositions that contain nucleic acid(s) comprising Tcb1-male (Tcb1-m) coding and/or regulatory sequences, such as expression cassettes, vectors, transformed host cells, and genetically engineered plants. In further embodiments, the disclosure provides compositions that contain nucleic acid(s) comprising Tcb1-f coding and/or regulatory sequences and nucleic acid(s) comprising Tcb1-m coding and/or regulatory sequences, such as expression cassettes, vectors, transformed host cells, and genetically engineered plants. The disclosure also provides methods of making and using the genetically engineered plants, that incude for example, controlling plant pollination and overcoming species pollination barriers, conferring cross-incompatibility and self-incompatibility in plants, and isolating a breeding population of plants, such as a genetically modified plant population.

In some embodiments, the disclosure relates to genetically engineered plants containing Tcb1-f coding and/or regulatory sequences and/or Tcb1-m coding and/or regulatory sequences. In some embodiments, the genetically engineered plants contain Tcb1-f coding sequences. In some embodiments, the genetically engineered plants contain Tcb1-f regulatory sequences. In some embodiments, the genetically engineered plants contain Tcb1-f coding sequences, but do not contain Tcb1-m coding sequences. In some embodiments, the genetically engineered plants contain Tcb1-m coding sequences. In some embodiments, the genetically engineered plants contain Tcb1-m regulatory sequences. In some embodiments, genetically engineered plants contain Tcb1-m coding sequences, but do not contain Tcb1-f coding sequences. In some embodiments, the genetically engineered plants contain Tcb1-f coding sequences and/or Tcb1-m coding sequences and exhibit the phenotype of cross-incompatibility. In some embodiments, the genetically engineered plants contain Tcb1-f coding sequences and/or Tcb1-m coding sequences and exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In some embodiments, the genetically engineered plants comprise Tcb1-f coding and/or regulatory sequences, and/or Tcb1-m coding and/or regulatory sequences and further comprise ZmPME10-1 coding and/or regulatory sequences, GA1-female (GA1-f) coding and/or regulatory sequences, and/or GA1-male (GA1-m) coding and/or regulatory sequences. In some embodiments, the genetically engineered plants comprise Tcb1-f coding sequences and/or Tcb1-m coding sequences and further comprise ZmPME10-1 coding sequences, GA1-female (GA1-f) coding sequences, and/or GA1-encoding sequences. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In some embodiments, the genetically engineered plants comprise Tcb1-f coding and/or regulatory sequences, and/or Tcb1-m coding and/or regulatory sequences and further comprise ZmPME10-1 coding and/or regulatory sequences, GA2-female (GA2-f) coding and/or regulatory sequences, and/or GA2-male (GA2-m) coding and/or regulatory sequences. In some embodiments, the genetically engineered plants comprise Tcb1-f coding sequences and/or Tcb1-m coding sequences and further comprise ZmPME10-1 coding sequences, GA2-f coding sequences, and/or GA2-m coding sequences. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In some embodiments, the genetically engineered plants comprise Tcb1-f coding and/or regulatory sequences, and/or Tcb1-m coding and/or regulatory sequences and further comprise ZmPME10-1 coding and/or regulatory sequences, GA1-f coding and/or regulatory sequences, and/or GA1-mcoding and/or regulatory sequences. GA2-f coding and/or regulatory sequences, and/or GA2-m coding and/or regulatory sequences. In some embodiments, the genetically engineered plants comprise Tcb1-f coding sequences and/or Tcb1-m coding sequences and further comprise ZmPME10-1 coding sequences, GA1-f coding sequences, and/or GA2-m, GA2-f coding sequences, and/or GA2-m coding sequences. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In additional embodiments, the genetically engineered plants comprise Tcb1-f coding and/or regulatory sequences, Tcb1-m coding and/or regulatory sequences and further comprise ZmPME10-1 coding and/or regulatory sequences, GA1-f coding and/or regulatory sequences, GA1-mcoding and/or regulatory sequences GA2-f coding and/or regulatory sequences, and/or GA2-m coding and/or regulatory sequences. In further embodiments, the genetically engineered plants comprise Tcb1-f coding sequences and/or Tcb1-m coding sequences and further comprise ZmPME10-1 coding sequences, GA1-f coding sequences, GA1-mcoding sequences, GA2-f coding sequences, and/or GA2-m coding sequences. In some embodiments, the genetically engineered plants exhibit the phenotype of cross-incompatibility. In some embodiments, the genetically engineered plants exhibit the phenotype of self-incompatibility. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In additional embodiments, the disclosure provides genetically engineered plants containing GA2-f coding and/or regulatory sequences, and/or GA2-m coding and/or regulatory sequences. In some embodiments, the genetically engineered plants contain GA2-f coding sequences. In some embodiments, the genetically engineered plants contain GA2-f regulatory sequences. In some embodiments, the genetically engineered plants contain GA2-f coding sequences, but do not contain GA2-m coding sequences. In some embodiments, the genetically engineered plants contain GA2-m coding sequences. In some embodiments, the genetically engineered plants contain GA2-m regulatory sequences. In some embodiments, genetically engineered plants contain GA2-m coding sequences, but do not contain GA2-f coding sequences. In some embodiments, the genetically engineered plants contain GA2-f coding sequences and/or GA2-m coding sequences and exhibit the phenotype of cross-incompatibility. In some embodiments, the genetically engineered plants contain GA2-f coding sequences and/or GA2-m coding sequences and exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In some embodiments, the genetically engineered plants comprise GA2-f coding and/or regulatory sequences, and/or GA2-m coding and/or regulatory sequences and further comprise ZmPME10-1 coding and/or regulatory sequences, GA1-f coding and/or regulatory sequences, and/or GA1-mcoding and/or regulatory sequences. In some embodiments, the genetically engineered plants comprise GA2-f coding sequences, GA2-m coding sequences and further comprise ZmPME10-1 coding sequences, GA1-f coding sequences, and/or GA1-mcoding sequences. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In some embodiments, the genetically engineered plants comprise GA2-f coding and/or regulatory sequences, and/or GA2-m coding and/or regulatory sequences and further comprise Tcb1-female (Tcb1-f) coding and/or regulatory sequences, and/or Tcb1-mcoding and/or regulatory sequences. In some embodiments, the genetically engineered plants comprise GA2-f coding sequences and/or GA2-m coding sequences and further comprise Tcb1-f coding sequences, and/or Tcb1-mcoding sequences. In some embodiments, the genetically engineered plants exhibit the phenotype of cross-incompatibility. In some embodiments, the genetically engineered plants exhibit the phenotype of self-incompatibility. The disclosure also provides methods of making and using these genetically engineered plants that include for example, controlling plant pollination and overcoming species pollination barriers, conferring cross-incompatibility and self-incompatibility in plants, and isolating a breeding population of plants, such as a genetically modified plant population.

In one embodiment, the disclosure provides:
[1] an isolated nucleic acid(s) comprising a polynucleotide sequence selected from:
   (a) a polynucleotide sequence encoding a polypeptide comprising the amino acid sequence of SEQ ID NO:1 (Tcb1-m, fl) or SEQ ID NO:2 (Tcb1-m);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355, contiguous amino acids of SEQ ID NO:2;
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2, wherein the polypeptide has at least one Tcb1-m biological activity such as PME activity or the ability to bind ZmPME10-1 having the amino acid sequence of SEQ ID NO:21;
   (d) a polynucleotide sequence encoding a polypeptide comprising an amino acid sequence that is at least 98%, 98.5%, 99%, or 99.5% identical to the amino acid sequence of SEQ ID NO:2, wherein the polypeptide has at least one Tcb1-m biological activity such as PME activity or the ability to bind ZmPME10-1 having the amino acid sequence of SEQ ID NO:21;
   (e) a polynucleotide sequence encoding a polypeptide comprising the amino acid sequence of SEQ ID NO:5 (Tcb1-f fit aa) or SEQ ID NO:6 (Tcb1-f);
   (f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30 (Tcb1-f, mat), wherein the polypeptide has Tcb1-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (Tcb1-f), wherein the polypeptide has at least one Tcb1-f biological activity such as PME activity;
   (h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na); and/or
   (j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na);
[2] the nucleic acid(s) of [1], which
   (i) encodes the polypeptide of any one of (a)-(d), but does not encode the polypeptide of any one of (e)-(h);
   (ii) encodes the polypeptide of any one of (e)-(h), but does not encode the polypeptide of any one of (a)-(d);
   (iii) encodes the polypeptide of any one of (a)-(d), and the polypeptide of any one of (e) -(h);
   (iv) comprises the polynucleotide sequence of (i) but does not comprises the polynucleotide sequence of (j);
   (v) comprises the polynucleotide sequence of (j) but does not comprises the polynucleotide sequence of (i); and/or
   (vi) comprises the polynucleotide sequence of (i) and the polynucleotide sequence of (j);
[3] an isolated nucleic acid(s) comprising a polynucleotide sequence selected from:
   (a) a polynucleotide sequence encoding a polypeptide comprising the amino acid sequence of SEQ ID NO:25 (GA2-m, fl) or SEQ ID NO:26 (GA2-m, mat);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m, mat);
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m, mat), wherein the polypeptide has at least one GA2-m biological activity such as PME activity;
   (d) a polynucleotide sequence encoding a polypeptide comprising an amino acid sequence that is at least 98%, 98.5%, 99%, or 99.5% identical to the amino acid sequence of SEQ ID NO:26 (GA2-m, mat), wherein the polypeptide has at least one GA2-m biological activity such as PME activity;
   (e) a polynucleotide sequence encoding a polypeptide comprising the amino acid sequence of SEQ ID NO:29 (GA2-f, fl) or SEQ ID NO:30 (GA2-f, mat);
   (f) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f, mat), wherein the polypeptide has GA2-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO: 6 (GA2-f, mat), wherein the polypeptide has at least one GA2-f biological activity such as PME activity;
   (h) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na);
   (j) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:33;
   (k) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (l) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34;
[4] the nucleic acid(s) of [3], which
   (i) encodes the polypeptide of any one of (a)-(d), but does not encode the polypeptide of any one of (e)-(h);
   (ii) encodes the polypeptide of any one of (e)-(h), but does not encode the polypeptide of any one of (a)-(d);
   (iii) encodes the polypeptide of any one of (a)-(d), and the polypeptide of any one of (e)-(h);
   (iv) comprises the polynucleotide sequence of (i) but does not comprises the polynucleotide sequence of (j);
   (v) comprises the polynucleotide sequence of (j) but does not comprises the polynucleotide sequence of (i); and/or
   (vi) comprises the polynucleotide sequence of (i) and the polynucleotide sequence of (j);
[5] the nucleic acid(s) of any one of [1]-[4], which further comprises:
   (a) a nucleic acid comprising a polynucleotide sequence encoding a polypeptide comprising the amino acid sequence of SEQ ID NO:21 (ZmPME10-1, mat);
   (b) a polynucleotide sequence encoding a polypeptide comprising a fragment of a protein having the amino acid sequence of at least about 150, 200, 250 300, 350, 400, 450, 500, 550, 600, 650, 655, 660, 665, 670, 657, 676, 677, 678, 679, or 680, contiguous amino acids of SEQ ID NO:21, wherein the polypeptide has ZmPME10-1 biological activity such as the ability to bind Tcb1-m (SEQ ID NO:2);
   (c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind Tcb1-m (SEQ ID NO:2); and/or
   (d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 or SEQ ID NO:4; (Tcb1-m, fl, mat na))
[6] the nucleic acid(s) of any one of [1]-[5], which further comprises:
   (a1) a polynucleotide sequence encoding a polypeptide comprising the amino acid sequence of SEQ ID NO:13 (GA1-m, fl) or SEQ ID NO:14 (GA1-m, mat);
   (b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 having the amino acid sequence of SEQ ID NO:21;
   (c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21; and/or
   (d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
   (a2) a polynucleotide sequence encoding a polypeptide comprising the amino acid sequence of SEQ ID NO:17 (GA1-f, mat) or SEQ ID NO:18 (GA1-f, mat);
   (b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
   (c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
   (d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f prom, mat, full-length na);
[7] the nucleic acid(s) of any one of [1]-[6], which is operably linked to one, or more than one, promoter, optionally wherein the one, or more than one, promoter is a constitutive promoter or an inducible promoter;
[8] the nucleic acid(s) of [7], which is operably linked to one, or more than one, endogenous promoter or heterologous promoter, optionally wherein the nucleic acid(s) is operably linked to one, or more one, male specific promoter (e.g., a pollen-and pollen-tube specific promoter) or one, or more than one, female specific promoter (e.g., a corn silk specific promoter), or a fragment thereof;
[9] the nucleic acid(s) of [7] or [8] wherein the promoter comprises at least 200 nucleotides of a Tcb1-m promoter, a GA1-m promoter, Tcb1-f promoter, or a GA1-f promoter;
[10] the nucleic acid(s) of any one of [5]-[9], wherein the one, or more than one promoter comprises a polynucleotide sequence selected from:
   (a) a polynucleotide sequence comprising at least about 50, 60, 75, 100, 125, 150, 175, 200, 225, 260, 275, 300, 325, 350, 375, 400 or more nucleotides of SEQ ID NO:9 (Tcb1-m, prom);
   (b) a polynucleotide sequence that is at least 85%, 90%, 95%, 96%, 97%, 98%, or 99%, identical to the polynucleotide sequence of SEQ ID NO:9 (Tcb1-m, prom);
   (c) a polynucleotide sequence comprising at least about 50, 60, 75, 100, 125, 150, 175, 200, 225, 260, 275, 300, 325, 350, 375, 400 or more nucleotides of a GA1-m promoter;
   (d) a polynucleotide sequence comprising at least about 50, 60, 75, 100, 125, 150, 175, 200, 225, 260, 275, 300, 325, 350, 375, 400 or more nucleotides of a ZmPME10-1 promoter;
   (e) a polynucleotide sequence comprising at least about 50, 60, 75, 100, 125, 150, 175, 200, 225, 260, 275, 300, 325, 350, 375, 400 or more nucleotides of a Tcb1-f promoter;
   (f) a polynucleotide sequence comprising at least about 50, 60, 75, 100, 125, 150, 175, 200, 225, 260, 275, 300, 325, 350, 375, 400 or more nucleotides of SEQ ID NO:12 (GA1-f, prom); and/or
   (g) a polynucleotide sequence that is at least 85%, 90%, 95%, 96%, 97%, 98%, or 99%, identical to the polynucleotide sequence of SEQ ID NO:12 (GA1-f, prom);
[11] An expression cassette or vector comprising the nucleic acid(s) of any one of [1]-[10], optionally wherein the vector expresses a polypeptide with Tcb1-m, Tcb1-f, GA1-m, GA1-f, GA2-m, GA2-f, or ZmPME10-1, biological activity, such as PME activity;
[12] a host cell comprising the nucleic acid(s) of any one of [1]-[10] or the expression cassette or vector of [11];
[13] the host cell of [12], wherein the nucleic acid(s) is integrated into the DNA of the host cell;
[14] the host cell of [12] or [13], wherein the host cell is a bacterial, fungal, insect, animal, or plant cell;
[15] the host cell of any one of [12]-[14], wherein the host cell is a plant cell;
[16] the host cell of any one of [12]-[15], wherein the host cell is a monocot;
[17] the host cell of any one of [12]-[16], wherein the host cell is a maize cell;
[18] the host cell of any one of [12]-[16], wherein the host cell is a wheat cell, a rice cell, a barley cell, an oat cell, a proso millet cell, a rye cell, a turfgrass cell, a fescue cell, a sorghum cell, a sugarcane cell, or an algal cell;
[19] the host cell of any one of [12]-[15], wherein the host cell is a dicot, optionally wherein the cell is a soybean cell, canola cell, sunflower cell, a sugar beet cell, a quinoa cell, an alfalfa cell, or a cotton cell;
[20] a transgenic plant comprising the nucleic acid(s) of any one of [1]-[10], the expression cassette or vector of [11], or the host cell of any one of [12]-[19];
[21] a transgenic plant stably transformed with the isolated nucleic acid(s) of any one of [1]-[10], or the expression cassette or vector of [11];
[22] the transgenic plant of [21], wherein the transgenic plant is stably transformed with the nucleic acid of any one of [1](a)-(d) or [1](e)-(f);
[23] a transgenic plant of [21], wherein the transgenic plant is stably transformed with the nucleic acid of any one of [3](a)-(d) or [3](e)-(f);
[24] a method of conferring self-incompatibility of a plant, the method comprising:
   stably transforming a plant cell with an isolated nucleic(s) acid comprising a polynucleotide sequence selected from the group:
   (a1) a polynucleotide sequence encoding a polypeptide comprising the amino acid sequence of SEQ ID NO:6 (Tcb1-f);
   (b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
   (c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such PME activity;
   (d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity; and/or
   (e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   an isolated nucleic(s) acid comprising a polynucleotide sequence selected from the group:
   (a2) a polynucleotide sequence encoding a polypeptide comprising the amino acid sequence of SEQ ID NO:6 (Tcb1-f);
   (b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
   (c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
   (d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity; and/or
   (e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   regenerating or developing the transgenic plant from the plant cell comprising the isolated nucleic acid, and expressing the polynucleotide sequence in the plant, optionally, wherein the plant is not teosinte or does not express Tcb1-m;
[23] the method of [22], which further comprises using a Tcb1-m plant comprising a polynucleotide sequence encoding a polypeptide comprising the amino acid sequence of SEQ ID NO:2 (Tcb1-m) from a separate plant line (either a natural Tcb1-m line or a transgenic Tcb1-m line) to force cross-pollination;
[24] a method of conferring male sterility to a plant, the method comprising:
   stably transforming a plant cell with an isolated nucleic acid(s) comprising a polynucleotide sequence selected from:
   (a) a polynucleotide sequence encoding a polypeptide comprising the amino acid sequence of SEQ ID NO:6 (Tcb1-f);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
   (d) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity; and/or
   (e) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   regenerating or developing a plant from the transformed plant cell comprising the isolated nucleic acid, and expressing the polynucleotide sequence in the plant; optionally, wherein the polynucleotide sequence is operably associated with one, or more than one, constitutive or inducible promoter, or one, or more than one, male-specific promoter (e.g., a pollen and a pollen-tube specific promoter);
[25] a method of creating an isolated breeding population of plants, the method comprising:
   stably transforming a plant cell with an isolated nucleic acid(s) comprising a polynucleotide sequence selected from:
   (a) a polynucleotide sequence encoding a polypeptide comprising the amino acid sequence of SEQ ID NO:2 (Tcb1-m);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as PME activity or the ability to bind ZmPME10-1 having the amino acid sequence of SEQ ID NO:21 (ZmPME10-1, mat);
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
   (d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
   (e) a polynucleotide sequence encoding a polypeptide comprising the amino acid sequence of SEQ ID NO:6 (Tcb1-f, mat);
   (f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
   (h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na); and/or
   (j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter);
[26] a method for forming a substantially homogeneous population of plants of a predetermined hybrid variety of a crop which is capable of undergoing both self-pollination and cross-pollination comprising: the method comprising:
   stably transforming a plant cell with an isolated nucleic(s) acid comprising a polynucleotide sequence selected from the group:
      (a1) a polynucleotide sequence encoding a polypeptide comprising the amino acid sequence of SEQ ID NO:6 (Tcb1-f);
      (b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
      (c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such PME activity;
      (d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
      (e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   an isolated nucleic(s) acid comprising a polynucleotide sequence selected from the group:
      (a2) a polynucleotide sequence encoding a polypeptide comprising the amino acid sequence of SEQ ID NO:6 (Tcb1-f);
      (b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
      (c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
      (d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
      (e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   regenerating or developing a plant from the transformed plant cell comprising the isolated nucleic acid, and expressing the polynucleotide sequence in the plant, optionally, wherein the plant does not express Tcb1-m;
[27] a method of overcoming species barriers to enable fertilization and transfer of traits between species, the method comprising stably transforming a plant cell with an isolated nucleic acid encoding a polypeptide selected from the group:
   stably transforming a plant cell with an isolated nucleic acid(s) comprising a polynucleotide sequence selected from:
   (a) a polynucleotide sequence encoding a polypeptide comprising the amino acid sequence of SEQ ID NO:2 (Tcb1-m);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as PME activity or the ability to bind ZmPME10-1 having the amino acid sequence of SEQ ID NO:21;
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
   (d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
   (e) a polynucleotide sequence encoding a polypeptide comprising the amino acid sequence of SEQ ID NO:6 (Tcb1-f);
   (f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
   (h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na); and/or
   (j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
   regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter);
[28] a method of conferring self-incompatibility of a plant, the method comprising:
   stably transforming a plant cell with an isolated nucleic(s) acid comprising a polynucleotide sequence selected from the group:
   (a1) a polynucleotide sequence encoding a polypeptide comprising the amino acid sequence of SEQ ID NO:29 (GA2-f, fl) or SEQ ID NO:30 (GA2-f, mat);
   (b1) a polynucleotide sequence encoding a polypeptide comprising a fragment of a protein having the amino acid sequence of at least about 50, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
   (c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such PME activity;
   (d1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
   (e1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (f1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
   an isolated nucleic(s) acid comprising a polynucleotide sequence selected from the group:
   (a2) a polynucleotide sequence encoding a polypeptide comprising the amino acid sequence of SEQ ID NO:29 (GA2-f, fl) or SEQ ID NO:30 (GA2-f, mat);
   (b2) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
   (c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
   (d2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity
   (e2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (f2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
   regenerating or developing the transgenic plant from the plant cell comprising the isolated nucleic acid, and expressing the polynucleotide sequence in the plant, optionally, wherein the plant is not teosinte or does not express GA2-m;
[29] the method of [28], which further comprises using a GA2-m plant comprising a polynucleotide sequence encoding a polypeptide comprising the amino acid sequence of SEQ ID NO:26 (GA2-m, mat) from a separate plant line (either a natural GA2-m line or a transgenic GA2-m line) to force cross-pollination;
[30] a method of conferring male sterility to a plant, the method comprising:
stably transforming a plant cell with an isolated nucleic acid(s) comprising a polynucleotide sequence selected from:
   (a) a polynucleotide sequence encoding a polypeptide comprising the amino acid sequence of SEQ ID NO:29 (GA2-f, fl) SEQ ID NO:30 (GA2-f, mat);
   (b) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
   (d) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
   (e) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (f) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34;and
   regenerating or developing a plant from the transformed plant cell comprising the isolated nucleic acid, and expressing the polynucleotide sequence in the plant; optionally, wherein the polynucleotide sequence is operably associated with one, or more than one, constitutive or inducible promoter, or one, or more than one, male-specific promoter (e.g., a pollen and a pollen-tube specific promoter);
[31] a method of creating an isolated breeding population of plants, the method comprising:
   stably transforming a plant cell with an isolated nucleic acid(s) comprising a polynucleotide sequence selected from:
   (a) a polynucleotide sequence encoding a polypeptide comprising the amino acid sequence of SEQ ID NO:26 (GA2-m, mat);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m), wherein the polypeptide has at least one GA2-m biological activity such as PME activity;
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m), wherein the polypeptide has at least one GA2-m biological activity such as PME activity;
   (d) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m), wherein the polypeptide has at least one GA2-m biological activity such as PME activity;
   (e) a polynucleotide sequence encoding a polypeptide comprising the amino acid sequence of SEQ ID NO:29 (GA2-f, fl) or SEQ ID NO:30 (GA2-f, mat);
   (f) a polynucleotide sequence encoding a polypeptide comprising a fragment of a protein having the amino acid sequence of at least about 0, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
   (h) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na);
   (j) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:33;
   (k) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (l) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
   regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter);
[32] a method for forming a substantially homogeneous population of plants of a predetermined hybrid variety of a crop which is capable of undergoing both self-pollination and cross-pollination comprising: the method comprising:
   stably transforming a plant cell with an isolated nucleic(s) acid comprising a polynucleotide sequence selected from the group:
      (a1) a polynucleotide sequence encoding a polypeptide comprising the amino acid sequence of SEQ ID NO:29 (GA2-f, fl) or SEQ ID NO:30 (GA2-f, mat);
   (b1) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has GA2-f biological activity such as PME activity;
   (c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such PME activity;
   (d1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
   (e1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
   (f1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and an isolated nucleic(s) acid comprising a polynucleotide sequence selected from the group:
   (a2) a polynucleotide sequence encoding a polypeptide comprising the amino acid sequence of SEQ ID NO:29 (GA2-f, fl) or SEQ ID NO:30 (GA2-f, mat);
   (b2) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has GA2-f biological activity such as PME activity;
   (c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such as PME activity;
   (d2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
   (e2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
   (f2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and regenerating or developing a plant from the transformed plant cell comprising the isolated nucleic acid, and expressing the polynucleotide sequence in the plant, optionally, wherein the plant does not express GA2-m;
[33] a method of overcoming species barriers to enable fertilization and transfer of traits between species, the method comprising stably transforming a plant cell with an isolated nucleic acid encoding a polypeptide selected from the group:
   stably transforming a plant cell with an isolated nucleic acid(s) comprising a polynucleotide sequence selected from:
   (a) a polynucleotide sequence encoding a polypeptide comprising the amino acid sequence of SEQ ID NO:26 (GA2-m, mat);
   (b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m, mat);
   (c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m), wherein the polypeptide has at least one GA2-m biological activity such as PME activity;
   (d) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m), wherein the polypeptide has at least one GA2-m biological activity such as PME activity;
   (e) a polynucleotide sequence encoding a polypeptide comprising the amino acid sequence of SEQ ID NO:29 (GA2-f, fl) or SEQ ID NO:30 (GA2-f, mat);
   (f) a polynucleotide sequence encoding a polypeptide comprising a fragment of a protein having the amino acid sequence of at least about 50, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f, mat), wherein the polypeptide has GA2-f biological activity such as PME activity;
   (g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
   (h) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
   (i) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na);
   (j) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:33;
   (k) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
   (l) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
   regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter);
[34] an isolated nucleic acid comprising a polynucleotide sequence selected from the group:
   (a) the polynucleotide sequence of SEQ ID NO:9 (Tcb1-m, prom);
   (b) a polynucleotide sequence comprising at least 100 consecutive nucleotides of the polynucleotide sequence of SEQ ID NO:9;
   (c) a polynucleotide sequence that hybridizes to the complete complement of the polynucleotide sequence of (a) or (b) under stringent hybridization conditions; and
   (d) a polynucleotide sequence having at least 95% sequence identity to the polynucleotide sequences of (a) or (b);
[35] an isolated nucleic acid comprising a polynucleotide sequence selected from the group:
   (a) the polynucleotide sequence of SEQ ID NO:12 (GA1-f, prom);
   (b) a polynucleotide sequence comprising at least 100 consecutive polynucleotides of nucleotides 1 to 800 of SEQ ID NO:12;
   (c) a polynucleotide sequence that hybridizes to the complete complement of the polynucleotide sequence of (a or (b) under stringent hybridization conditions; and
   (d) a polynucleotide sequence having at least 95% sequence identity to the polynucleotide sequences of (a) or (b); and/or.
[36] an isolated nucleic acid comprising a polynucleotide sequence selected from the group:
   (a) the polynucleotide sequence of SEQ ID NO:10 (Tcb1-f, prom);
   (b) a polynucleotide sequence comprising at least 100, 125, 150, 175, 200, 225, 250, 300, 350, 375, 400, 450, 500, 750, 1,000, 1,250, 2,000, 2,500, 3,000, 4,000, or 5,000 consecutive polynucleotides of SEQ ID NO:10;
   (c) a polynucleotide sequence that hybridizes to the complete complement of the polynucleotide sequence of (a) or (b) under stringent hybridization conditions; and
   (d) a polynucleotide sequence having at least 95% sequence identity to the polynucleotide sequences of (a) or (b).

These and other embodiments, are set forth in more detail below.

### BRIEF DESCRIPTION OF THE DRAWINGS

FIGS. 1A-1C. Mixed pollination test of the Tcb1-s mutants. FIG. 1A depicts a scheme of the experiment: Two pollen donor lines and three pollen receiver lines were used. Pollen from a tcb1; R1-self color maize line (purple circles), produces purple kernels, while pollen from test plants (yellow circles) produces white or yellow kernels. Pollen from the two donors was mixed and put on three receiving ears: (Matsuoka et al., PNAS 99:6080-6084 (2002)) Tcb1-s ears to verify the Tcb1-male function from the KO line; (Kermicle et al., Maydica 35:399-408 (1990)) Tcb1-f(KO) ears to test the presence/absence of the female barrier in the knockout mutant individuals; and (Evans et al., Theoretical and Applied Genetics 103:259-265 (2001)) a maize (tcb1; r1) neutral ear to identify the ratio of viable pollen grains from the two donors in the mixture. (FIG. 1B) Ears from the three pollen recipients for the Tcb1-f(KO1)) test. (FIG. 1C) Ears from the three pollen recipients for the Tcb1-f(KO2) test. In both tests, pollen from the KO plants successfully fertilized the Tcb1-s ear (left ear in FIG. 1B and FIG. 1C), while the ears from KO plants showed no barrier to tcb1 maize pollen with a similar frequency of purple kernels on mutant ears and the neutral test ears (middle and right ears in FIG. 1B and FIG. 1C).

FIGS. 2A-B. Gene expression profiling identified Tcb1-female as a Tcb1-s candidate gene. RNA samples collected from silks of different genotypes was analyzed by RNA-Seq and RT-PCR. FIG. 2A: Tcb1-female gene structure is shown above the graph (solid line indicates single intron), and RNA-Seq read depth is shown for the Tcb1-female gene. FIG. 2B: Tcb1-female gene expression, compared to tubulin levels as a control, as measured by qRT-PCR in a variety of loss-of-function Tcb1-s lines: Tcb1-s, full strength Tcb1-s barrier line; Tcb1-f(KO1) and Tcb1-f(KO2), two loss-of-function alleles from a Mutator transposon mutagenesis; Tcb1-m, a spontaneous Tcb1-male only line; Tcb1-f:silent lineage1 and Tcb1-f:silent lineage2; and K452-13 and J456-13, two Tcb1-male only lines that lost Tcb1-s by recombination.

FIGS. 3A-3C. Reversion of Tcb1-female loss-of-function. FIG. 3A: Revertant ear (marked by asterisk in FIG. 3B and FIG. 3C) pollinated by a mix of tcb1; R1-sc and Tcb1-m; r1 pollen showing higher frequency of yellow kernels on the test ear than the tcb1 control ear. FIG. 3B: Results of mixed pollination tests on four genotypes: tcb1, Tcb1-s, Tcb1-female (loss-of-function(lof)) alleles, and Tcb1-f(lof) mop1 double mutants. The percentage of kernels from tcb1 pollen on the test ear are plotted on the x-axis and the percentage of kernels from tcb1 pollen on the control tcb1 ear on the y-axis. Equal percentages in the two ears indicates no barrier (line with slope=1). Red circles indicate ears with significantly fewer tcb1 kernels in the test ear vs. the control ear at p<0.0001 and blue circles at 0.0001<p<0.01. The loss-of-function line is indicated for each revertant. FIG. 3C Tcb1-female gene expression level and barrier strength. Barrier strength is expressed as the ratio of kernels from Tcb1-s vs tcb1 pollen on the test ear vs. the control tcb1 ear (Columns), with a fraction of 1.0 indicating no barrier and significantly higher values a functional barrier. Tcb1-female RNA levels are expressed as relative to the tubulin control gene (Orange Line).

FIG. 4. Expression of Tcb1-female in different Tcb1-s lines collected in different locations in Mexico (Kermicle et al., Genetics 172:499-506 (2006)). W22 is a standard maize tcb1 line, Tcb1-s:Col48703, Tcb1-s:109-4a, and Tcb1-s:207-5d are three independent collections of Zea mays ssp. mexicana tesointe lines; Tcb1-s:104-4a, a Zea mays ssp. parviglumis line; and DGF1222-2 from Maiz Dulce, an ancient Mexican maize sweet corn variety (Jones et al., Euphytica 209:63-69 (2016)).

FIGS. 5A and 5B. S1. Mix pollination testing of spontaneous Tcb1-male plant. FIG. 5A depicts the mix pollination testing scheme of a spontaneous Tcb1-male plant. FIG. 5B presents ears from the three pollen receivers for Tcb1-male plant test. Pollen from the Tcb1-male plant successfully fertilized the Tcb1-s ear and produced yellow kernels (left ear in FIG. 5B), while the ears from Tcb1-male plant had lost the barrier to block maize pollen as shown by the purple kernels produced by tcb1 pollen on the ears (middle ear in FIG. 5B).

FIG. 6. Presents the result of a PCR-based assay using a dCAPS (Derived Cleaved Amplified Polymorphic Sequence) marker to test presence/absence of Tcb1-female in recombinants from the mapping population. The marker was designed such that only the PCR amplicon from the Tcb1 genomic DNA (T), but not the unspecific PCR product amplified from the maize genomic DNA (t) would be cut by the enzyme Hae III after PCR and enzyme digestion. The results indicate that Tcb1-female maps to the tcb1 locus.

FIG. 7. PCR detection of Tcb1-female in two Tcb1-f(KO) mutants. PCR primers spanning the possible mutation site in the Tcb1-f(KO1) was designed and tested on the Tcb1-s (WT), Tcb1-f(KO1) and Tcb1-f(KO2). Using Tcb1-f(KO1) genomic DNA as template failed to produce amplicon. Thus confirming that there was a disruption within the coding region of Tcb1-female in KO1, In contrast, in Tcb1-f(KO2), Tcb1-female was fully assembled, consistent with the PCR data that the coding region is present.

FIG. 8. Whole genome sequencing identified a Hopscotch retrotransposon insertion in the first exon in Tcb1-f(KO1), close to the site where Tcb1-female expression drops sharply. PCR spanning both ends of the insertion confirmed the insertion event and the border sequences. Black bases, Tcb1-female gene sequence; Red bases, retrotransposon sequence; Blue bases, bases duplicated from the left border leading the retrotransposon sequence.

FIG. 9. Partial DNA alignment of the Tcb1-female gene intron between the different Tcb1-s lines Collections 109-4a, 48703 and 207-5d, ssp. mexicana teosintes; Collection104-4a, ssp. parviglumis teosinte; DGF1222, a traditional maize Dulce sweetcorn variety from Mexico. One Single Nucleotide Polymorphism (SNP) in the intron separates these lines into two groups: one group including the parviglumis line (Col104-4a) and one mexicana line (Col109-4a), and the other group including two mexicana lines (Col48703 and Col207-5d) and the Maiz Dulce line.

FIG. 10. Alignment of full-length Tcb1-female (SEQ ID NO:5) and full-length GA1-female (SEQ ID NO:17) sequences show that the two pectin methylesterases (PMEs) differ in nine amino acids. Putative signal sequences are underscored.

FIG. 11. Phylogenic tree of mature pectin methylesterase (PME) enzymes (predicted pre and pro domains removed) of Arabidopsis PME proteins and predicted PME proteins encoded by cross-incompatibility loci of Zea mays.

FIG. 12. Alignment of full-length Tcb1-male (SEQ ID NO:1) and full-length GA1-male (SEQ ID NO:13) sequences show that the two pectin methylesterases (PMEs) differ in eleven amino acids. Putative signal sequences are underscored.

FIG. 13. Alignment of full-length GA1-male (SEQ ID NO:13) and full-length GA2-male (SEQ ID NO:25) sequences. The GA1-m and GA2-m sequences share 59% identity. Putative signal sequences are underscored.

FIG. 14. Alignment of full-length GA1-female (SEQ ID NO:17) and full-length GA2-female (SEQ ID NO:29) sequences. The GA1-f and GA2-f sequences share 55% identity. Putative signal sequences are underscored.

### DETAILED DESCRIPTION

The present disclosure will now be described in more detail with reference to the accompanying drawings, in which preferred embodiments, provided herein are shown. The subject matter encompassed by the disclosure can, however, be embodied in different forms and should not be construed as limited to the embodiments, set forth herein. Rather, these embodiments, are provided so that this disclosure will be thorough and complete, and will fully convey the scope provided herein to those skilled in the art.

Unless the context indicates otherwise, it is specifically intended that the various features provided herein described herein can be used in any combination.

Moreover, the present disclosure also contemplates that in some embodiments, provided herein, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this disclosure belongs. The terminology used in the description provided herein is for the purpose of describing particular embodiments, only and is not intended to be limiting provided herein.

All publications, patent applications, patents, and other references mentioned herein are incorporated by reference in their entirety.

### Definitions

As used herein, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed subject matter. See, In re Herz, 537 F.2d 549, 551-52 (CCPA 1976); see also MPEP § 2111.03. Thus, the term “consisting essentially of” when used in a claim or elsewhere in the disclosure is not intended to be interpreted to be equivalent to “comprising.”

The term “modulate” (and grammatical variations) refers to an increase or decrease.

As used herein, the terms “increase,” “increases,” “increased,” “increasing” and similar terms indicate an elevation of at least about 25%, 50%, 75%, 100%, 150%, 200%, 300%, 400%, 500% or more.

As used herein, the terms “reduce,” “reduces,” “reduced,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

As used herein, the term “heterologous” means foreign, exogenous, non-native and/or non-naturally occurring.

As used herein, “homologous” means native. For example, a homologous polynucleotide sequence or amino acid sequence is a polynucleotide sequence or amino acid sequence naturally associated with a host cell into which it is introduced, and a homologous promoter sequence is the promoter sequence that is naturally associated with a coding sequence, and the like.

The terms “nucleic acid,” “polynucleotide” and “nucleotide sequence” can be used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid can be double-stranded or single-stranded. The present disclosure further provides a nucleic acid comprising a polynucleotide sequence that is the complement (which can be either a full complement or a partial complement) of a nucleic acid sequence provided herein (e.g., a polynucleotide sequence comprising a Tcb1-m or Tcb1-f promoter element and/or is the complement of a Tcb1-m or Tcb1-f coding sequence provided herein). Polynucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage

The nucleic acids provided herein are optionally isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide can exist in a purified form or can exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides provided herein can be considered to be “isolated.”

Further, in some embodiments, an “isolated” nucleic acid contains a polynucleotide sequence (e.g., DNA or RNA) that is not immediately contiguous with polynucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism (e.g., maize), but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a polynucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid or polynucleotide refers to a nucleic acid containing a polynucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

The term “fragment,” as applied to a nucleic acid will be understood to mean a nucleic acid comprising a polynucleotide sequence of reduced length relative to the reference or full-length polynucleotide sequence and comprising and/or consisting of contiguous polynucleotides from the reference or full-length polynucleotide sequence. Such a fragment can be, where appropriate, included in a larger polynucleotide of which it is a constituent. In some embodiments, such fragments can comprise, consist essentially of, and/or consist of oligonucleotides having a length of at least about 8, 10, 12, 15, 16, 17, 18, 19, 20, 25, 30, 35, 40, 45, 50, 75, 100, 125, 150, 175, 200, 225, 250, 275, 300, 325, 350, 375, 400, 405, 410, 425, 450, 455, 460, 475, 500, 505, 510, 515 or 520, or more, contiguous nucleotides from the reference or full-length polynucleotide sequence (e.g., SEQ ID NOS:3, 4, 7, 9, 10, 12, 19, 20, 23, 24, 27, 28, 31, 32, 33, 34), as long as the fragment is shorter than the reference or full-length polynucleotide sequence. In representative embodiments, the fragment is a biologically active polynucleotide sequence, as that term is described herein.

A “biologically active” polynucleotide, or a polynucleotide having a “biological activity” is one that substantially retains at least one biological activity normally associated with the wild-type polynucleotide sequence, for example, a polynucleotide having the ability to drive transcription of an operatively associated coding sequence. In particular embodiments, the “biologically active” polynucleotide substantially retains all of at least one biological activity possessed by the unmodified corresponding sequence. By “substantially retains” biological activity, it is meant that the polynucleotide retains at least about 50%, 60%, 75%, 85%, 90%, 95%, 97%, 98%, 99%, or more, of the biological activity of the native polynucleotide (and can even have a higher level of activity than the native polynucleotide). For example, a biologically active promoter element is able to control, regulate and/or enhance the expression of a polynucleotide sequence operably associated with the promoter. Methods of measuring expression of a polynucleotide sequence are well known in the art and include Northern blots, RNA run-on assays and methods of measuring the presence of an encoded polypeptide (e.g., antibody based methods or visual inspection in the case of a reporter polypeptide).

Two polynucleotide sequences are said to be “substantially identical” to each other when they share at least 95%, 97%, 98%, 99% or even 100% sequence identity. Two polynucleotide sequences can also be considered to be substantially identical when the two sequences hybridize to each other under stringent conditions. A nonlimiting example of “stringent” hybridization conditions include conditions represented by a wash stringency of 50% Formamide with 5× Denhardt's solution, 0.5% SDS and 1× SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. (1993). In some representative embodiments, two polynucleotide sequences considered to be substantially identical hybridize to each other under highly stringent conditions. Generally, highly stringent hybridization and wash conditions are selected to be about 5° C. lower than the thermal melting point (Tm) for the specific sequence at a defined ionic strength and pH.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

A “conservative amino acid substitution” is one in which one amino acid residue is replaced with another amino acid residue having a similar side chain. Families of amino acid residues having similar side chains have been defined in the art, including, for example, basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides provided herein do not abrogate the biological activity (e.g., PME activity) of the polypeptide containing the amino acid sequence. Methods of identifying nucleotide and amino acid conservative substitutions which do not eliminate biological activity (e.g., PME activity) binding are well-known in the art (see, e.g., Brummell et al., Biochem. 32:1180-1187 (1993); Kobayashi et al., Protein Eng. 12(10):879-884 (1999); and Burks et al., PNAS 94:412-417 (1997)).

As is known in the art, a number of different programs can be used to identify whether a nucleic acid has sequence identity or an amino acid sequence has sequence identity or similarity to a known sequence. Sequence identity or similarity can be determined using standard techniques known in the art, including, but not limited to, the local sequence identity algorithm of Smith & Waterman, Adv. Appl. Math. 2, 482 (1981), by the sequence identity alignment algorithm of Needleman et al., J. Mol. Biol. 48,443 (1970), by the search for similarity method of Pearson & Lipman, PNAS 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng et al., J. Mol. Evol. 35:351-360 (1987); the method is similar to that described by Higgins et al., CABIOS 5:151-153 (1989).

Another example of a useful algorithm is the BLAST algorithm, described in Altschul et al., J. Mol. Biol. 215:403-410, (1990) and Karlin et al., PNAS 90:5873-5787 (1993). A particularly useful BLAST program is the WU-BLAST-2 program which was obtained from Altschul et al., Methods Enzymol. 266:460-480 (1996); blast.wustl/edu/blastl READMEhtml. WU-BLAST-2 uses several search parameters, which are preferably set to the default values. The parameters are dynamic values and are established by the program itself depending upon the composition of the particular sequence and composition of the particular database against which the sequence of interest is being searched; however, the values can be adjusted to increase sensitivity.

An additional useful algorithm is gapped BLAST as reported by Altschul et al., Nucleic Acids Res. 25:3389-3402 (1997).

The CLUSTAL program can also be used to determine sequence similarity. This algorithm is described by Higgins et al., Gene 73:237 (1988); Higgins et al., CABIOS 5:151-153 (1989); Corpet et al., Nucleic Acids Res. 16:10881-90 (1988); Huang et al., CABIOS 8:155-65 (1992); and Pearson et al., Meth. Mol. Biol. 24:307-331 (1994).

The alignment can include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

As used herein a “chimeric nucleic acid” or “chimeric polynucleotide” comprises a promoter operably linked to a polynucleotide sequence of interest that is heterologous to the promoter (or vice versa). In particular embodiments, the “chimeric nucleic acid,” “chimeric polynucleotide sequence” or “chimeric polynucleotide” comprises a Tcb1-f promoter element (e.g., SEQ ID NO:10, or a fragment thereof) operably associated with a heterologous polynucleotide sequence of interest to be transcribed. In other representative embodiments, the “chimeric nucleic acid,” “chimeric polynucleotide sequence” or “chimeric polynucleotide” comprises a Tcb1-f coding sequence operably associated with a heterologous promoter. In additional particular embodiments, the “chimeric nucleic acid,” “chimeric polynucleotide sequence” or “chimeric polynucleotide” comprises a Tcb1-m promoter element operably associated with a heterologous polynucleotide sequence of interest to be transcribed. In other representative embodiments, the “chimeric nucleic acid,” “chimeric polynucleotide sequence” or “chimeric polynucleotide” comprises a Tcb1-m coding sequence operably associated with a heterologous promoter.

A “promoter” is a polynucleotide sequence that controls or regulates the transcription of a polynucleotide sequence (i.e., a coding sequence) that is operatively associated with the promoter. The coding sequence can encode a polypeptide and/or a functional RNA Typically, a “promoter” refers to a polynucleotide sequence that contains a binding site for RNA polymerase II and directs the initiation of transcription. In general, promoters are found 5′, or upstream, relative to the start of the coding region of the corresponding coding sequence. The promoter region can comprise other elements that act as regulators of gene expression. These include a TATA box consensus sequence, and often a CAAT box consensus sequence (Breathnach et al., Annu. Rev. Biochem. 50:349 (1981)). In plants, the CAAT box can be substituted by the AGGA box (Messing et al., (1983) in Genetic Engineering of Plants, Kosuge et al. (eds.), Plenum Press, pp. 211-227). The promoter region, including all the ancillary regulatory elements, typically contain between about 100 and 1000 nucleotides, but can be as long as 2 kb, 3 kb, 4 kb or longer in length.

Promoters according to the present disclosure can function as constitutive and/or inducible regulatory elements. The promoters can also be endogenous and/or heterologous. Non-limiting examples of constitutive promoters include cestrum virus promoter (cmp) (U.S. Pat. No. 7,166,770), an actin promoter (e.g., the rice actin 1 promoter; Wang et al., Mol. Cell. Biol. 12:3399-3406 (1992); as well as U.S. Pat. No. 5,641,876), Cauliflower Mosaic Virus (CaMV) 35S promoter (Odell et al., Nature 313:810-812 (1985)), CaMV 19S promoter (Lawton et al., Plant Mol. Biol. 9:315-324 (1987)), an opine synthetase promoter (e.g., nos, mas, ocs, etc.; (Ebert et al., PNAS 84:5745-5749 (1987)), Adh promoter (Walker et al., PNAS 84:6624-6629 (1987)), sucrose synthase promoter (Yang & Russell, PNAS 87:4144-4148 (1990)), and a ubiquitin promoter.

In addition, promoters functional in plastids can be used. Non-limiting examples of such promoters include the bacteriophage T3 gene 9 5′ UTR and other promoters disclosed in U.S. Pat. No. 7,579,516. Other promoters useful with the present disclosure include but are not limited to the S-E9 small subunit RuBP carboxylase promoter and the Kunitz trypsin inhibitor gene promoter (Kti3).

In some embodiments, inducible promoters are used in the embodiments, of the present disclosure. Examples of inducible promoters useable with the present disclosure include, but are not limited to, tetracycline repressor system promoters, Lac repressor system promoters, copper-inducible system promoters, salicylate-inducible system promoters (e.g., the PRla system), glucocorticoid-inducible promoters (Aoyama et al., Plant J. 11:605-612 (1997)), and ecdysone-inducible system promoters. Other non-limiting examples of inducible promoters include ABA- and turgor-inducible promoters, the auxin-binding protein gene promoter (Schwab et al., Plant J. 4:423-432 (1993)), the UDP glucose flavonoid glycosyl-transferase promoter (Ralston et al., Genetics 119:185-197 (1988)), the 1VIPI proteinase inhibitor promoter (Cordero et al., Plant J. 6:141-150 (1994)), the glyceraldehyde-3-phosphate dehydrogenase promoter (Kohler et al., Plant Mol. Biol. 29:1293-1298 (1995); Martinez et al., J. Mol. Biol. 208:551-565 (1989); and Quigley et al., J. Mol. Evol. 29:412-421 (1989)) the benzene sulphonamide-inducible promoters (U.S. Pat. No. 5,364,780) and the glutathione S-transferase promoters. Likewise, one can use any appropriate inducible promoter described in Gatz et al., Current Opinion Biotechnol. 7:168-172 (1996) and Gatz et al., Annu. Rev. Plant Physiol. Plant Mol. Biol. 48:89-108 (1997).

In some embodiments, the disclosure provides compositions that comprise a heterologous promoter operably linked to the provided polynucleotide sequences. The heterologous promoter can be any suitable heterologous promoter known in the art (including bacterial, yeast, fungal, insect, mammalian, and plant promoters). In particular embodiments, the promoter is a promoter for expression in plants. In some embodiments, the heterologous promoter is a promoter for expression in a monocot plant. In further embodiments, the heterologous promoter is selected from: ZmUbi1 (Ubiquitin), Act1 (Actin), OsTubA1, (Tubulin), OsCc1 (Cytochrome c), rubi3 (polyubiquitin), APX (ascorbate peroxidase), SCP1, PGD1 (phosphogluconate dehydrogenase), R1G1B (early drought induced protein) and EIF5 (translation initiation factor). In some embodiments, the heterologous promoter is a promoter for expression in a dicot plant. In further embodiments, the heterologous promoter is a CsVMV (cassava vein mosaic virus) or ScBV (sugarcane bacilliform badnavirus) promoter. Other suitable promoters include promoters from viruses that infect the host plant including, but not limited to, promoters isolated from Dasheen mosaic virus, Chlorella virus (e.g., the Chlorella virus adenine methyltransferase promoter; Mitra et al., Plant Molecular Biology 26:85 (1994)), tomato spotted wilt virus, tobacco rattle virus, tobacco necrosis virus, tobacco ring spot virus, tomato ring spot virus, cucumber mosaic virus, peanut stump virus, alfalfa mosaic virus, and the like.

In some embodiments, the promoter preferentially expresses a polynucleotide provided herein in one or more male tissues of a plant. Male-tissue promoters useful in driving the expression of one or more polynucleotide sequences provided herein are known in the art. Such promoters may include, but are not limited to promoters that are expressed after tetrad formation within the maturing pollen grain, the mature pollen grain, during pollen germination, and/or within the pollen tube. In some embodiments, the male-tissue promoter is a member selected from: the PG47 promoter (see, e.g., U.S. Pat. No. 5,412,085); the Mpcbp promoter (see, e.g., Reddy et al., J. Biol. Chem. 275(45):35457-70 (2000)); the MS45 promoter (see, e.g., U.S. Pat. No. 6,037,523, sequence identifier numbers 1 and 2); the 5126 promoter (see, e.g., U.S. Pat. No. 5,837,851); the BS7promoter (see, e.g., Intl. Publ. No. WO 2002/063021); the SGB6 promoter (see, e.g., U.S. Pat. No. 5,470,359); the G9 promoter (see, e.g., U.S. Pat. No. 5,837,850); the SB200 promoter (see, e.g., Intl. Publ. No. WO 2002/26789), or a biologically active fragment of anyone of the above (e.g., a fragment that drives expression in the male tissue of a plant).

In some embodiments, the male-tissue promoter is a member selected from: the ZmC5 promoter (see, e.g., U.S. Publ. No. US20040045053A1, Int. Publ. No. W01999042587, and/or Wakeley et al., Plant Mol. Biol. 37:187-192 (1998)); the Zm908 promoter (see, e.g., Peng et al., Front Plant Sci. 8:685 (2017)); the ZmMADS2 promoter (see, e.g., Schreiber et al., Plant Physiol. 134(3):1069-79 (2004)); the Zm13 promoter (see, e.g., Hamilton et al., Plant Mol Biol. 38:663-669 (1998)), and U.S. Pat. No. 5,086,169); the Zmprol promoter (see, e.g., Kovar et al., The Plant Cell 12:583-598 (2000)); the ZmPSK1 promoter (see, e.g., Lorbiecke et al., J. Exp. Bot. 56(417):1805-1819(2005)); the Zmabp1 or Zmabp2 promoter (see, e.g., Lopez et al., PNAS 93:7415-7420 (1996)); the maize Ms45 promoter (see, e.g., U.S. Pat. No. 6,037,523); a pollen-specific promoter having the sequence of any one of the sequences corresponding to sequence identifier numbers 2-6 of U.S. Pat. No. 5,412,085; a pollen specific promoter described in Fearing et al., Mol. Breeding 3:169-176 (1997)); or a biologically active fragment of anyone of the above (e.g., a fragment that drives expression in a plant male tissue (e.g., a pollen tube)).

In some embodiments, the male-tissue promoter is a member selected from: the tomato LAT52 promoter (see, e.g., Twell et al., Development 109:705-713 (1990)); the Brassica Bp19 promoter (see, e.g., Albani et al., PMB 16:501-513 (1991)); the Brassica Bca9 promoter (see, e.g., Lee et al., Plant Cell Rep. 22:268-273 (2003)); the tobacco NTP303 promoter (see, e.g., Weterings et al., Plant J. 8:55-63 (1991)); the wheat TaPSG719 promoter (see, e.g., Chen et al., Mol Biol. Rep. 37:737-744 (2010)); the tobacco NTPp13 promoter (see, e.g., Yang et al., Genetika 46:458-463 (2010)); the 5126 promoter described in U.S. Pat. Nos. 5,837,851 and 5,689,051; the SF3 promoter described in U.S. Pat. No. 6,452,069; the BS92-7 promoter described in Intl. Publ. No. WO 02/063021; a SGB6 regulatory element described in U.S. Pat. No. 5,470,359; the TA29 promoter (see, e.g., Koltunow et al., Plant Cell 2:1201-1224 (1990), Goldberg et al., Plant Cell 5:1217-1229 (1993), and U.S. Pat. No. 6,399,856); the type 2 metallothionein-like gene promoter (see, e.g., Charbonnel-Campaa et al., Gene 254:199-208 (2000); the PG47 promoter (see, e.g., U.S. Pat. No. 5,412,085; U.S. Pat. No. 5,545,546; Plant J 3(2):261-271 (1993)); the Mpcbp promoter (Reddy et al., J. Biol. Chem. 275(45):35457-70 (2000)); a promoter of one of the pollen-specific genes described in Khurana et al. (Critical Rev. Plant Science 31:359-390 (2012)); or a biologically active fragment of anyone of the above (e.g., a fragment that drives expression in a plant male tissue (e.g., a pollen tube)).

In some embodiments, the promoter preferentially expresses a polynucleotide provided herein in one or more female tissues of a plant. Female-tissue promoters useful in driving the expression of one or more polynucleotide sequences provided herein are known in the art. In some embodiments, the female-tissue promoter is a member selected from: a corn silk promoter disclosed in CA2481504, a promoter disclosed in U.S. Pat. No. 6,515,204 (see, e.g., nucleotides 1-1986 of the sequences corresponding to sequence identifier numbers SEQ ID NO:1; SEQ ID NO 2), the promoter of the maize silk expressed gene disclosed in Xu et al., PLoS One 8(1): (2013), and a promoter disclosed in U.S. Pat. No. 6,392,123 (e.g., nucleotides 1 to 1390 of the sequence corresponding to sequence identifier number 11).

In some embodiments, the expression cassettes provided herein can further comprise enhancer elements and/or tissue preferred elements in combination with the promoter. In some embodiments, the expression cassette comprises a constitutive S35 promoter operably associated with a polynucleotide sequence encoding Tcb1-m having the amino acid sequence of SEQ ID NO:2. In some embodiments, the expression cassette comprises a constitutive S35 promoter operably associated with a polynucleotide sequence encoding Tcb1-m comprising the amino acid sequence of SEQ ID NO:2.

In some embodiments, the expression cassettes provided herein can further comprise enhancer elements and/or tissue preferred elements in combination with the promoter. In some embodiments, the expression cassette comprises a constitutive S35 promoter operably associated with a polynucleotide sequence encoding Tcb1-f having the amino acid sequence of SEQ ID NO:6. In some embodiments, the expression cassette comprises a constitutive S35 promoter operably associated with a polynucleotide sequence encoding Tcb1-f comprising the amino acid sequence of SEQ ID NO:6.

In some embodiments, the expression cassettes provided herein can further comprise enhancer elements and/or tissue preferred elements in combination with the promoter. In some embodiments, the expression cassette comprises a constitutive S35 promoter operably associated with a polynucleotide sequence encoding GA2-m having the amino acid sequence of SEQ ID NO:25. In some embodiments, the expression cassette comprises a constitutive S35 promoter operably associated with a polynucleotide sequence encoding GA2-m comprising the amino acid sequence of SEQ ID NO:26.

In some embodiments, the expression cassettes provided herein can further comprise enhancer elements and/or tissue preferred elements in combination with the promoter. In some embodiments, the expression cassette comprises a constitutive S35 promoter operably associated with a polynucleotide sequence encoding GA2-f having the amino acid sequence of SEQ ID NO:29. In some embodiments, the expression cassette comprises a constitutive S35 promoter operably associated with a polynucleotide sequence encoding GA2-f comprising the amino acid sequence of SEQ ID NO:30.

By “operably linked” or “operably associated” as used herein, it is meant that the indicated elements are functionally related to each other, and are also generally physically related. For example, a promoter is operatively linked or operably associated to a coding sequence (e.g., polynucleotide sequence of interest) if it controls the transcription of the sequence. Thus, the term “operatively linked” or “operably associated” as used herein, refers to polynucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

The term “expression cassette” as used herein includes a polynucleotide sequence encoding a polypeptide to be expressed and sequences controlling its expression such as a promoter and optionally an enhancer sequence, including any combination of cis-acting transcriptional control elements. The sequences controlling the expression of the gene, i.e. its transcription and the translation of the transcription product, are commonly referred to as regulatory unit. Most parts of the regulatory unit are located upstream of coding sequence of the gene and are operably linked thereto. The expression cassette may also contain a downstream 3′ untranslated region comprising a polyadenylation site. The regulatory unit can be operably linked to the coding sequence to be expressed, i.e. transcription unit, or separated therefrom by intervening DNA such as for example by the 5′-untranslated region of the heterologous gene. Preferably the expression cassette is flanked by one or more suitable restriction sites in order to enable the insertion of the expression cassette into a vector and/or its excision from a vector. Thus, the expression cassette provided herein can be used for the construction of an expression vector, in particular a plant expression vector. The expression cassette provided herein may comprise one or more e.g., two, three or even more non-translated genomic DNA sequences downstream of a plant promoter or fragments thereof, and/or one or more e.g. two, three or even more non-translated genomic DNA sequences upstream of a plant promoter or fragments thereof. The expression cassette may be in the form of a vector, and can be used, alone or in combination with other expression cassettes or vectors.

In some embodiments, the disclosure provides an expression cassette comprising a Tcb1-f nucleic acid provided herein operably associated with a promoter. In some embodiments, the Tcb1-f nucleic acid is operably associated with a Tcb1-f promoter sequence or fragment thereof. In other embodiments, the Tcb1-f nucleic acid nucleic acid is operably associated with a heterologous promoter or fragment thereof.

In some embodiments, the disclosure provides an expression cassette comprising a Tcb1-m nucleic acid provided herein operably associated with a promoter. In some embodiments, the Tcb1-m nucleic acid is operably associated with a Tcb1-m promoter sequence or fragment thereof. In other embodiments, the Tcb1-m nucleic acid nucleic acid is operably associated with a heterologous promoter or fragment thereof

The disclosure also provides an expression cassette comprising a Tcb1-f promoter or fragment thereof, optionally in operable association with a polynucleotide sequence of interest. The expression cassette can further have a plurality of restriction sites for insertion of a polynucleotide sequence of interest to be operably linked to the regulatory regions. In some embodiments, the Tcb1-f promoter or fragment thereof is operably associated with a nucleic acid encoding a Tcb1-f polypeptide provided herein. In some embodiments, the Tcb1-f promoter or fragment is operably associated with a heterologous polynucleotide sequence of interest. In some embodiments, the Tcb1-f promoter or fragment thereof is operably associated with a heterologous polynucleotide sequence of interest. In some embodiments, the Tcb1-f promoter or fragment thereof is operably associated with a polynucleotide sequence encoding Tcb1-f (SEQ ID NO:6) or GA1-f (SEQ ID NO:18).

In an additional embodiment, the disclosure provides an expression cassette comprising a Tcb1-m promoter or fragment thereof, optionally in operable association with a polynucleotide sequence of interest. The expression cassette can further have a plurality of restriction sites for insertion of a polynucleotide sequence of interest to be operably linked to the regulatory regions. In some embodiments, the Tcb1-m promoter or fragment thereof is operably associated with a nucleic acid encoding a Tcb1-m polypeptide provided herein. In some embodiments, the Tcb1-m promoter or fragment is operably associated with a heterologous polynucleotide sequence of interest. In some embodiments, the Tcb1-m promoter or fragment thereof is operably associated with a heterologous polynucleotide sequence of interest. In some embodiments, the Tcb1-m promoter comprises the polynucleotide sequence of SEQ ID NO:9 or a fragment thereof. In further embodiments, the Tcb1-m promoter (SEQ ID NO:9) or fragment thereof is operably associated with a polynucleotide sequence encoding Tcb1-f (SEQ ID NO:6), GA1-f (SEQ ID NO:18), Tcb1-m (SEQ ID NO:2), GA1-m (SEQ ID NO:14), or ZmPME10-1 (SEQ ID NO:21).

In some embodiments, the disclosure provides an expression cassette comprising a GA2-f nucleic acid provided herein operably associated with a promoter. In some embodiments, the GA2-f nucleic acid is operably associated with a Tcb1-f promoter sequence or fragment thereof. In other embodiments, the GA2-f nucleic acid nucleic acid is operably associated with a heterologous promoter or fragment thereof.

In some embodiments, the disclosure provides an expression cassette comprising a GA2-m nucleic acid provided herein operably associated with a promoter. In some embodiments, the GA2-m nucleic acid is operably associated with a Tcb1-f promoter sequence or fragment thereof. In other embodiments, the GA2-m nucleic acid nucleic acid is operably associated with a heterologous promoter or fragment thereof.

The disclosure also provides an expression cassette comprising a Tcb1-f promoter or fragment thereof, optionally in operable association with a polynucleotide sequence of interest. The expression cassette can further have a plurality of restriction sites for insertion of a polynucleotide sequence of interest to be operably linked to the regulatory regions. In some embodiments, the Tcb1-f promoter or fragment thereof is operably associated with a nucleic acid encoding a Tcb1-f polypeptide provided herein. In some embodiments, the Tcb1-f promoter or fragment is operably associated with a heterologous polynucleotide sequence of interest. In some embodiments, the Tcb1-f promoter or fragment thereof is operably associated with a heterologous polynucleotide sequence of interest. In some embodiments, the Tcb1-f promoter or fragment thereof is operably associated with a polynucleotide sequence encoding Tcb1-f (SEQ ID NO:6) or GA1-f (SEQ ID NO:18).

In an additional embodiment, the disclosure provides an expression cassette comprising a Tcb1-f promoter or fragment thereof, optionally in operable association with a polynucleotide sequence of interest. The expression cassette can further have a plurality of restriction sites for insertion of a polynucleotide sequence of interest to be operably linked to the regulatory regions. In some embodiments, the Tcb1-f promoter or fragment thereof is operably associated with a nucleic acid encoding a Tcb1-f polypeptide provided herein. In some embodiments, the Tcb1-f promoter comprises the polynucleotide sequence of SEQ ID NO:10 or a fragment thereof. In further embodiments, the Tcb1-f promoter (SEQ ID NO:10) or fragment thereof is operably associated with a polynucleotide sequence encoding Tcb1-f (SEQ ID NO:6), GA1-f (SEQ ID NO:18), Tcb1-m (SEQ ID NO:2), GA1-m (SEQ ID NO:14), or ZmPME10-1 (SEQ ID NO:21).

“Nucleotide sequence of interest” refers to any polynucleotide sequence which, when introduced into a plant, confers upon the plant a desired characteristic such as antibiotic resistance, virus resistance, insect resistance, disease resistance, or resistance to other pests, herbicide tolerance, abiotic stress resistance (e.g., drought tolerance, salt tolerance, tolerance to waterlogging and/or submergence stress, and the like), improved nutritional value, improved performance in an industrial process or altered reproductive capability. The “nucleotide sequence of interest” can encode a polypeptide or functional RNA (e.g., a regulatory RNA). For example, the “nucleotide sequence of interest” can be one that is transferred to plants for the production of a polypeptide (e.g., an enzyme, hormone, growth factor or antibody) for commercial production.

A “heterologous polynucleotide sequence” or “heterologous polynucleotide sequence of interest” as used herein is a coding sequence that is heterologous to an associated (e.g., operably linked) promoter sequence referenced herein (e.g., a Tcb1-f promoter or Tcb1-m promoter or fragment thereof (i.e., is not the native sequence corresponding to the expressed protein of interest). The heterologous polynucleotide sequence can encode a polypeptide or a functional RNA. A “heterologous promoter” is a promoter that is heterologous to the polynucleotide sequence with which it is operatively associated. For example, a Tcb1-f coding sequence can be operatively associated with a heterologous promoter (e.g., a promoter that is not the native Tcb1-f promoter sequence with which the Tcb1-f coding sequence is associated in its naturally occurring state).

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

“Wild-type” polynucleotide sequence or amino acid sequence refers to a naturally occurring (“native”) or endogenous polynucleotide sequence (including a cDNA corresponding thereto) or amino acid sequence.

A “vector” is any nucleic acid molecule for the cloning of and/or transfer of a nucleic acid into a cell. A vector can be a replicon to which another polynucleotide can be attached to allow for replication of the attached polynucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art can be used to manipulate, deliver and express polynucleotides. Vectors can be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more heterologous polynucleotide sequences (e.g., transgenes), e.g., two, three, four, five or more heterologous polynucleotide sequences.

Viral vectors have been used in a wide variety of gene delivery applications in cells, as well as living animal subjects. Plant viral vectors that can be used include, but are not limited to, Agrobacterium tumefaciens, Agrobacterium rhizogenes and geminivirus vectors. Non-viral vectors include, but are not limited to, plasmids, liposomes, electrically charged lipids (cytofectins), nucleic acid-protein complexes, and biopolymers. In addition to a nucleic acid of interest, a vector can also comprise one or more regulatory regions, and/or selectable markers useful in selecting, measuring, and monitoring nucleic acid transfer results (e.g., delivery to specific tissues, duration of expression, etc.).

As used herein, the term “polypeptide” encompasses both peptides and proteins (including fusion proteins), unless indicated otherwise.

A “fusion protein” is a polypeptide produced when two heterologous polynucleotide sequences or fragments thereof coding for two (or more) different polypeptides not found fused together in nature are fused together in the correct translational reading frame.

The polypeptides provided herein are optionally “isolated.” An “isolated” polypeptide is a polypeptide that, by the hand of man, exists apart from its native environment and is therefore not a product of nature. An isolated polypeptide can exist in a purified form or can exist in a non-native environment such as, for example, a recombinant host cell. The recombinant polypeptides provided herein can be considered to be “isolated.”

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material. In representative embodiments, the isolated polypeptide is a recombinant polypeptide produced using recombinant nucleic acid techniques. In some embodiments, the polypeptide is a fusion protein.

The term “fragment,” as applied to a polypeptide, will be understood to mean an amino acid of reduced length relative to a reference polypeptide or the full-length polypeptide (e.g., Tcb1-f) and comprising, and/or consisting of a sequence of contiguous amino acids from the reference or full-length polypeptide. Such a fragment can be, where appropriate, included as part of a fusion protein of which it is a constituent. In some embodiments, such fragments can comprise, consist essentially of, and/or consist of polypeptides having a length of at least about 50, 75, 100, 125, 150, 200, 250 300, 350, 400, 450, 500, 550, 600, 650, 655, 660, 665, 666, 667, 668, 669, 670, 671, or 672 contiguous amino acid residues from the reference or full-length polypeptide (e.g., SEQ ID NO: 1 or 2, SEQ ID NO:5 or 6, SEQ ID NO:13 or 14, SEQ ID NO:17 or 18, or SEQ ID NO:21 or 22), as long as the fragment is shorter than the reference or full-length polypeptide. In representative embodiments, the fragment is biologically active, as that term is defined herein.

A “biologically active” polypeptide or a polypeptide having a “biological activity” is one that substantially retains at least one biological activity normally associated with the wild-type polypeptide, such as, PME activity under physiological conditions in vitro, or the ability to bind ZmPME10-1 under physiological conditions in vitro. In particular embodiments, the “biologically active” polypeptide substantially retains at least one of the biological activities possessed by the unmodified (wild-type) sequence. By “substantially retains” biological activity, it is meant that the polypeptide retains at least about 50%, 60%, 75%, 85%, 90%, 95%, 97%, 98%, 99%, or more, of a biological activity of the native polypeptide, such as PME activity or the ability to bind ZmPME10-1. Methods of measuring PME and/or the ability of a polypeptide to bind another protein are known in the art.

“Pectin Methylesterase (PME) activity” or “PME activity” as used herein, is the ability to catalyze the cleavage of methylester groups from pectin. PMEs (pectin methylesterases) catalyse the demethylesterification of plant cell wall polygalacturonans such as pectins Pectin deesterification catalyzed by PMEs frees carboxyl groups on pectin chains, which in turn promotes pectin crosslinking with Ca²⁺ and the stiffening of plant cell walls that increase the mechanical strength, but decrease the plasticity of plant cells such as the apical region of pollen tube walls that is essential for pollen tube growth. Methods of measuring PME activity are described herein and known in the art.

PME activity cleaves methyl groups from galacturonic acid residues in pectin chains and results in the formation of carboxyl groups that leads to a drop in pH. In one embodiment, the PME activity of a sample (e.g., a plant sample) is be detected using a pH indicator test. In one embodiment, the PME activity of a sample (e.g., a plant sample) is be detected using a methyl red indicator test. The pH indicator methyl red changes color at pH drop from yellow (pH 6.2) to pink (pH 4.2). In some embodiments, the assay contains 1 ml 0.5 Grindsted™ Pectin 1450 (Danisco Ingredients, Danisco A/S)) solubilized in 0.15 M NaCl pH 7 and 25 ul sample. Samples that indicate a positive methyl red test after 10 minute incubation at 30° C. are then further measured by a titration method (Versteeg et al., Wiss. Technol. 11:267-274 (1978), the contents of which are herein incorporated by reference in its entirety). In some embodiments, the titration method assay for PME activity is performed using 10 ml 0.5 lime pectin (Grindsted™ Pectin 1450 (Danisco Ingredients, Danisco A/S) solubilized in 0.15 M NaCI pH 6.8 and 10-100 ul sample. Titration is performed with 0.02 M NaOH and the reaction is measured at room temperature. An automatic titrator can be used (Versteeg et al. Lebensmittel-Wiss. U. Technol. 11: 267-274 (1978)). In another embodiment, PME activity is quantified using a titration technique in which different amounts of sample fractions are added to a reaction solution (0.01 U/μL alcohol oxidase, 0.5% citrus pectin, 200 mM sodium phosphate, pH 6.2) and incubated at room temperature for 30 min. Fluoral-P (Sigma) is then added to a final concentration of 4 mg/mL. After incubation at room temperature for 5 min, the fluorescent intensity is measured in an Enspire reader (PerkinElmer) at 510 nm upon excitation at 405 nm. The released methanol amounts that represent PME activity are quantified based on the standard curve established using a methanol gradient. Methods and reagents for detecting and quantifying PME are known in the art. See, e.g., Intl. Publ. No. WO 00/78982, the contents of which are herein incorporated by reference in its entirety.

In another embodiment, pectin methylesterase activity is assayed according to the methods set forth in Lionetti, Front. Plant Sci. 6:331 (2015), the contents of which are herein incorporated by reference in its entirety). Briefly, agarose plates are prepared by pouring 50 mL of media containing 0.1% (w/v) citrus pectin (Sigma), 1% (w/v) LE agarose (USB), 12.5 mM citric acid, and 50 mM Na₂HPO₄, pH 6.5, into 12-cm² petri dishes. After solidification, the plates are punched using a capillary tube at equal distance. Equal volumes of the eluent fractions are loaded into the punched wells and incubated at 30° C. for 10-16 h. The plates are then stained with 0.05% (w/v) ruthenium red (R2751, Sigma) for 30 min and destained by rinsing with distilled water. The stained circle size indicated PME activity.

Methods for determining the ability of proteins to bind one another are known in the art. In some embodiments, the ability of a protein to bind ZmPME10-1 (SEQ ID NO:21), is determined using a yeast two hybrid system or a firefly luciferase complementation assay.

“Introducing”, “introduction” (and similar terms) in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide construct, or as separate polynucleotide-constructs, and can be located on the same or different polynucleotide constructs. Accordingly, these nucleic acid molecules can be introduced into plant cells in a single transformation event, in separate transformation events, or, e.g., as part of a breeding protocol. The nucleic acid molecules can be DNA or RNA and can be single stranded or double stranded.

The term “transformation” as used herein refers to the introduction of a heterologous nucleic acid into a cell. Transformation of a cell can be stable or transient. Thus, a transgenic cell (e.g., plant cell), plant tissue, plant part and/or plant provided herein can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants. In particular embodiments, the polynucleotide is stably integrated into the genome of the cell by methods of site directed integration (e.g., using a zinc-finger nuclease, engineered or native meganuclease, TALE-endonuclease, or an RNA-guided endonuclease such as Cas9 or Cpf1)).

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated polynucleotide. In representative embodiments, the recombinant or isolated polynucleotide sequence is stably integrated into the genome of the plant (e.g., into a chromosome or as a stable extra-chromosomal element), so that it is passed on to subsequent generations of the cell or plant. In particular embodiments, the polynucleotide is stably integrated into the genome of the cell by site directed integration.

The term “plant” as used herein, includes reference to an immature or mature whole plant, including a plant that has been detasseled or from which seed or grain has been removed. Seed or embryo that will produce the plant is also considered to be the plant.

The term “plant part,” as used herein, includes but is not limited to reproductive tissues (e.g., petals, sepals, stamens, silk, stigma, pistils, receptacles, anthers, pollen, flowers, fruits, flower bud, ovules, seeds, embryos, nuts, kernels, grain, ears, pericarp, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell provided herein can be in the form of an isolated single cell or can be a cultured cell or can be a part of a higher-organized unit such as, for example, a plant tissue (including callus) or a plant organ. Any plant (or groupings of plants, for example, into a genus or higher order classification) can be employed in practicing the present disclosure including monocots or dicots.

Exemplary transgenic plants provided herein include, but are not limited to, corn (Zea mays), canola (Brassica napus, Brassica rapa ssp.), alfalfa (Medicago saliva), rice (Oryza sativa), rape (Brassica napus), rye (Secale cereale), sorghum (Sorghum bicolor, Sorghum vulgare), sunflower (Hehanthus annuus), wheat (Triticum aestivum), soybean (Glycine max), tobacco (Nicotiana tobacum), cotton (Gossypium hirsutum), sugar beets (Beta vulgaris), oats, barley, turfgrasses (e.g., for ornamental, recreational or forage purposes), and biomass grasses (e.g., switchgrass and miscanthus). Turfgrass which can be employed in practicing the compositions and methods provided herein include but are not limited to zoysia grasses, bent grasses, fescue grasses, bluegrasses, St. Augustine grasses, bermudagrasses, buffalo grasses, rye grasses, and orchard grasses.

In particular embodiments, the transgenic plant provided herein is corn (Zea mays). In some embodiments, the transgenic plant is wheat (Tritium aestivum), or rice (Oryza sativa). In particular embodiments, the transgenic plant provided herein is a member selected from wheat (Tritium aestivum), corn (Zea mays) and rice (Oryza sativa). In particular embodiments, the transgenic plant is corn (Zea mays). In other embodiments, the transgenic plant is wheat (Tritium aestivum) or rice (Oryza sativa). In an additional embodiment, the transgenic plant is alfalfa or sunflower. In a particular embodiment, the transgenic plant is soybean (Glycine max).

In particular embodiments, the transgenic plant is an algae.

### Nucleic Acids

In some embodiments, the disclosure provides compositions that contain isolated nucleic acid(s) comprising a pollination barrier factor coding sequence (e.g., a Tcb1-f coding sequence, Tcb1-m coding sequence, GA2-f coding sequence, and/or GA2-m coding sequence).

In additional embodiments, the provided compositions contain isolated nucleic acid(s) comprising a polynucleotide sequence encoding a fragment or variant of a pollination barrier factor such as Tcb1-f, Tcb1-m, GA2-f, and/or GA2-m. In further embodiments, the encoded fragment or variant has at least one biological activity of the reference pollination barrier factor protein e.g., pectin methylesterase (PME) activity).

In some embodiments, the provided compositions comprise a nucleic acid encoding a fragment of a pollination barrier factor that has the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of the reference pollination barrier factor. In some embodiments, the reference pollination barrier factor is a member selected from: Tcb1-f, Tcb1-m, GA1-f, GA1-m GA2-f, GA2-m, and/or ZmPME10-1. In further embodiments, the encoded fragment has at least one biological activity of the reference pollination barrier factor protein (e.g., PME activity or the ability to bind ZmPME10-1 having the amino acid sequence of SEQ ID NO:21).

In some embodiments, the provided compositions comprise a nucleic acid encoding ft polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference pollination barrier factor In some embodiments, the reference pollination barrier factor is a member selected from: Tcb1-f, Tcb1-m, GA1-f, GA1-m GA2-f, GA2-m, and/or ZmPME10-1. In further embodiments, the encoded polypeptide has at least one biological activity of the reference pollination barrier factor protein (e.g., PME activity or the ability to bind ZmPME10-1 having the amino acid sequence of SEQ ID NO:21).

In additional embodiments, the provided compositions comprise a nucleic acid encoding a polypeptide comprising an amino acid sequence that is at least 98%, 98.5%, 99%, or 99.5% identical to the sequence of a reference pollination barrier factor. In some embodiments, the reference pollination barrier factor is a member selected from: Tcb1-f, Tcb1-m, GA1-f, GA1-m GA2-f, GA2-m, and/or ZmPME10-1. In further embodiments, the encoded polypeptide has at least one biological activity of the reference pollination barrier factor protein (e.g., PME activity or the ability to bind ZmPME10-1 having the amino acid sequence of SEQ ID NO:21) SEQ ID NO:2 (Tcb1-m). In further embodiments, the encoded polypeptide has at least one Tcb1-m biological activity such as PME activity or the ability to bind ZmPME10-1 having the amino acid sequence of SEQ ID NO:21.

In some embodiments, the nucleic acid(s) provided herein is operably linked with one, or more than one, constitutive or inducible promoter or a fragment thereof, or one, or more than one, male specific promoter (e.g., a pollen/pollen-tube specific promoter) or a fragment thereof, or one, or more than one, female specific promoter (e.g., a corn silk specific promoter), or a fragment thereof. The disclosure also provides expression cassettes comprising the nucleic acids.

Vectors comprising the nucleic acids and expression cassettes provided herein as also encompassed by the disclosure, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a cosmid, BAC, or YAC. In particular embodiments, the vector is not a BAC or a YAC. In particular embodiments, the vector is not a BAC or a cosmid. In further embodiments, embodiments, the vector is not a BAC.

The disclosure also provides host cells comprising the nucleic acids, expression cassettes and vectors provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, fungal (e.g., yeast), insect and/or mammalian cells. In representative embodiments, the host cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algale cell. In some embodiments, the plant cell is a dicot cell. In further embodiments, the host cell is a soybean cell, canola cell, sunflower cell, sugar beet cell, quinoa cell, alfalfa cell, or a cotton cell.

In additional embodiments, the disclosure provides compositions that contain the provided nucleic acid(s) comprising Tcb1-m coding and/or promoter sequences, and/or Tcb1-f coding and/or promoter sequences, such as expression cassettes and vectors, transformed host cells, and genetically engineered plants. In some embodiments, the disclosure provides compositions that contain nucleic acid(s) comprising Tcb1-m coding and/or promoter sequences, such as expression cassettes, vectors, transformed host cells, and genetically engineered plants. In some embodiments, the disclosure provides compositions that contain nucleic acid(s) comprising Tcb1-f coding and/or promoter sequences and nucleic acid(s) comprising Tcb1-m coding and/or regulatory sequences, such as expression cassettes, vectors, transformed host cells, and genetically engineered plants.

In some embodiments, the disclosure provides genetically engineered plants containing the Tcb1-f nucleic acid(s) provided herein including for example, expression cassettes and vectors comprising the nucleic acids. In some embodiments, the disclosure relates to genetically engineered plants containing the Tcb1-m nucleic acid(s) provided herein including for example, expression cassettes and vectors comprising the nucleic acids. In some embodiments, the disclosure relates to genetically engineered plants containing the Tcb1-f nucleic acid(s) and Tcb1-m nucleic acid(s) provided herein including for example, expression cassettes and vectors comprising the Tcb1-f nucleic acid(s) and Tcb1-m nucleic acid(s). In additional embodiments, the genetically engineered plants further comprise ZmPME10-1 nucleic acid(s) provided herein including for example, expression cassettes and vectors comprising the ZmPME10-1 nucleic acids. In some embodiments, the genetically engineered plants further comprise GA1-f nucleic acid(s) provided herein including for example, expression cassettes and vectors comprising the GA1-f nucleic acids. In some embodiments, the genetically engineered plants further comprise GA1-m nucleic acid(s) provided herein including for example, expression cassettes and vectors comprising the GA1-m nucleic acids. In some embodiments, the genetically engineered plants further comprise GA1-f nucleic acid(s) and GA1-m nucleic acid(s) provided herein including for example, expression cassettes and vectors comprising the GA1-f nucleic acid(s) and GA1-m nucleic acid(s). Methods of making and using the plants are also encompassed by the disclosure.

### Tcb1-m Nucleic Acid(s), Expression Constructs and Vectors

The term “Tcb1-m nucleic acid(s)” and grammatical variants thereof, as used herein is intended to encompass Tcb1-m nucleic acids provided herein (e.g., polynucleotides encoding the amino acid sequence of SEQ ID NO:1 and SEQ ID NO:2) and fragments and variants thereof. In some embodiments, the disclosure provides nucleic acids that encode Tcb1-m polypeptides. In representative embodiments, the nucleic acids are isolated. In particular embodiments, the Tcb1-m fragments or variants have at least one Tcb1-m biological activity such as PME activity or the ability to bind ZmPME10-1 having the amino acid sequence of SEQ ID NO:21.

In some embodiments, the disclosure provides an isolated nucleic acid(s) comprising a polynucleotide sequence that encodes the amino acid sequence of SEQ ID NO:2. In some embodiments, the polynucleotide sequence comprises the sequence of SEQ ID NO:4. In further embodiments, the isolated nucleic acid(s) comprises a polynucleotide sequence that encodes the amino acid sequence of SEQ ID NO:1. In yet further embodiments, the polynucleotide sequence comprises the sequence of SEQ ID NO:3. Polypeptides encoded by the polynucleotides are also encompassed by the disclosure.

In additional embodiments, the disclosure provides an isolated Tcb1-m nucleic acid(s) comprising a polynucleotide sequence that encodes a fragment or variant of the amino acid sequence of SEQ ID NO:2. In some embodiments, the polynucleotide sequence encodes a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355, contiguous amino acids of SEQ ID NO:2 (Tcb1-m). In particular embodiments, the encoded polypeptide has at least one Tcb1-m biological activity such as PME activity or the ability to bind ZmPME10-1 having the amino acid sequence of SEQ ID NO:21. Polypeptides encoded by the polynucleotides are also encompassed by the disclosure.

In some embodiments, the disclosure provides an isolated Tcb1-m nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m). In some embodiments, the encoded polypeptide comprises an amino acid sequence having a total of one, two, three, four, five, six, or seven, conservative amino acid substitutions from a reference amino acid sequence of SEQ ID NO:2. In some embodiments, the encoded polypeptide comprises an amino acid sequence having a total of one, two, three, four, five, six, or seven, non-conservative amino acid substitutions from a reference amino acid sequence of SEQ ID NO:2. In other embodiments, the encoded polypeptide comprises an amino acid sequence having conservative and non-conservative amino acid substitutions from a reference amino acid sequence of SEQ ID NO:2. In particular embodiments, the encoded polypeptide has at least one Tcb1-m biological activity such as PME activity or the ability to bind ZmPME10-1 having the amino acid sequence of SEQ ID NO:21. Polypeptides encoded by the polynucleotides are also encompassed by the disclosure.

In some embodiments, the provided isolated Tcb1-m nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:2. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:2. In particular embodiments, the encoded polypeptide has at least one Tcb1-m biological activity such as PME activity or the ability to bind ZmPME10-1 having the amino acid sequence of SEQ ID NO:21. Polypeptides encoded by the nucleic acid(s) are also encompassed by the disclosure.

In some embodiments, the provided Tcb1-m isolated nucleic acid(s) encodes a polypeptide comprising an amino acid sequence that is at least 98%, 98.5%, 99%, or 99.5% identical to the amino acid sequence of SEQ ID NO:2 (Tcb1-m). In further embodiments, the encoded polypeptide is at least 98%, 98.5%, 99%, or 99.5% identical to the amino acid sequence of SEQ ID NO:1. In particular embodiments, the encoded polypeptide has at least one Tcb1-m biological activity such as PME activity or the ability to bind ZmPME10-1 having the amino acid sequence of SEQ ID NO:21. Polypeptides encoded by the nucleic acid(s) are also encompassed by the disclosure.

In additional embodiments, the disclosure provides an isolated Tcb1-m nucleic acid(s) comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na), or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:4 (Tcb1-m, mat na), or the complete complementary strand thereto.

In additional embodiments, the disclosure provides an isolated nucleic acid(s) comprising a Tcb1-m promoter or fragment or variant thereof. In some embodiments, the nucleic acid comprises the polynucleotide sequence of SEQ ID NO:9 (Tcb1-m, prom). In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In additional embodiments, a Tcb1-m nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the Tcb1-m nucleic acid is operably linked with male specific promoter. Suitable male specific promoters are described herein and/or otherwise known in the art. In further embodiments, the Tcb1-m nucleic acid is operably linked with a pollen/pollen-tube specific promoter. Suitable male pollen/pollen-tube specific promoters are described herein and/or otherwise known in the art. In yet further embodiments, the Tcb1-m nucleic acid is operably linked with a maize pollen/pollen-tube specific promoter. Suitable maize pollen/pollen-tube specific promoters are described herein and/or otherwise known in the art.

In some embodiments, the Tcb1-m nucleic acid is operably linked with a polynucleotide sequence comprising a Tcb1-m promoter. In some embodiments, the Tcb1-m nucleic acid is operably linked with a polynucleotide sequence comprising at least, 75, 100, 125, 150, 175, 200, 225, or 250 contiguous nucleotides of a the sequence of a Tcb1-m promoter. In some embodiments, the Tcb1-m nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the Tcb1-m nucleic acid is operably linked with a polynucleotide sequence comprising a GA1-m promoter. In some embodiments, the Tcb1-m nucleic acid is operably linked with a GA1-m polynucleotide sequence comprising at least, 75, 100, 125, 150, 175, 200, 225, or 250 contiguous nucleotides of a GA1-m promoter.

In some embodiments, the Tcb1-m nucleic acid is operably linked with a polynucleotide sequence comprising a GA2-m promoter. In some embodiments, the Tcb1-m nucleic acid is operably linked with a GA2-m polynucleotide sequence comprising at least, 75, 100, 125, 150, 175, 200, 225, or 250 contiguous nucleotides of a GA2-m promoter.

In some embodiments, the Tcb1-m nucleic acid is operably linked with a female specific promoter. Suitable female specific promoters are described herein and/or otherwise known in the art. In further embodiments, the Tcb1-m nucleic acid is operably linked with a maize female tissue specific promoter. Suitable maze female tissue specific promoters are described herein and/or otherwise known in the art. In yet further embodiments, the Tcb1-m nucleic acid is operably linked with a maize corn silk specific promoter specific promoter. Suitable maize corn silk specific promoter are described herein and/or otherwise known in the art.

In some embodiments, the Tcb1-m nucleic acid is operably linked with a polynucleotide sequence comprising a Tcb1-m promoter. In some embodiments, the Tcb1-m nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the Tcb1-m nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the Tcb1-m nucleic acid is operably linked with a polynucleotide sequence comprising a ZmPME10-1 promoter. In some embodiments, the Tcb1-m nucleic acid is operably linked with a ZmPME10-1 polynucleotide sequence comprising at least, 75, 100, 125, 150, 175, 200, 225, or 250 contiguous nucleotides of a ZmPME10-1 promoter.

In some embodiments, the Tcb1-m nucleic acid is operably linked with a polynucleotide sequence comprising a GA2-f promoter. In some embodiments, the Tcb1-m nucleic acid is operably linked with a GA2-f polynucleotide sequence comprising at least, 75, 100, 125, 150, 175, 200, 225, or 250 contiguous nucleotides of a GA2-f promoter.

In some embodiments, the Tcb1-m nucleic acid is operably linked with a Tcb1 polynucleotide sequence. In some embodiments, the Tcb1-m nucleic acid is operably linked with a GA1-m polynucleotide sequence comprising at least, 75, 100, 125, 150, 175, 200, 225, or 250 contiguous nucleotides of the sequence of SEQ ID NO: 10 or SEQ ID NO:12. In other embodiments, the nucleic acid is operably linked with a nucleic acid sequence that hybridizes with the polynucleotide sequence of SEQ ID NO: 10, or the reverse complementary sequence thereof, under stringent hybridization conditions.

In some embodiments, the Tcb1-m nucleic acid is operably linked with a polynucleotide sequence comprising a GA1-f promoter. In some embodiments, the Tcb1-m nucleic acid is operably linked with a polynucleotide sequence comprising at least, 75, 100, 125, 150, 175, 200, 225, or 250 contiguous nucleotides of a the sequence of a GA1-f promoter. In some embodiments, the Tcb1-m nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

Expression cassette(s) comprising the Tcb1-m nucleic acid(s) are also provided.

The disclosure further provides vectors comprising the Tcb1-m nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

In additional embodiments, the disclosure provides host cells comprising the Tcb1-m nucleic acid(s) nucleic acids, expression cassettes and vectors comprising the Tcb1-m nucleic acids provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### Tcb1-f Nucleic Acids, Expression Constructs and Vectors

The term “Tcb1-f nucleic acid(s)” and grammatical variants thereof, as used herein is intended to encompass Tcb1-f nucleic acids provided herein (e.g., polynucleotides encoding the amino acid sequence of SEQ ID NO:5 and SEQ ID NO:6) and fragments and variants thereof. In additional embodiments, the disclosure provides nucleic acids that encode Tcb1-f polypeptides. In representative embodiments, the nucleic acids are isolated. In particular embodiments, the Tcb1-f fragments or variants have at least one Tcb1-f biological activity such as PME activity.

In some embodiments, the disclosure provides an isolated Tcb1-f nucleic acid(s) comprising a polynucleotide sequence that encodes the amino acid sequence of SEQ ID NO:5. In some embodiments, the polynucleotide sequence comprises the sequence of SEQ ID NO:6. In further embodiments, the isolated nucleic acid(s) comprises a polynucleotide sequence that encodes the amino acid sequence of SEQ ID NO:5. In yet further embodiments, the polynucleotide sequence comprises the sequence of SEQ ID NO:7. Polypeptides encoded by the polynucleotides are also encompassed by the disclosure.

In additional embodiments, the disclosure provides an isolated Tcb1-f nucleic acid(s) comprising a polynucleotide sequence that encodes a fragment or variant of the amino acid sequence of SEQ ID NO:6. In some embodiments, the polynucleotide sequence encodes a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f). In particular embodiments, the encoded polypeptide has at least one Tcb1-f biological activity such as PME activity. Polypeptides encoded by the nucleic acids are also encompassed by the disclosure.

In some embodiments, the disclosure provides an isolated Tcb1-f nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f). In some embodiments, the encoded polypeptide comprises an amino acid sequence having a total of one, two, three, four, five, six, or seven, conservative amino acid substitutions from a reference amino acid sequence of SEQ ID NO:6. In some embodiments, the encoded polypeptide comprises an amino acid sequence having a total of one, two, three, four, five, six, or seven, non-conservative amino acid substitutions from a reference amino acid sequence of SEQ ID NO:6. In other embodiments, the encoded polypeptide comprises an amino acid sequence having conservative and non-conservative amino acid substitutions from a reference amino acid sequence of SEQ ID NO:6. In particular embodiments, the encoded polypeptide has at least one Tcb1-f biological activity such as PME activity. Polypeptides encoded by the polynucleotides are also encompassed by the disclosure.

In some embodiments, the provided isolated Tcb1-f nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:6. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:5. In particular embodiments, the encoded polypeptide has at least one Tcb1-f biological activity such as PME. Polypeptides encoded by the nucleic acid(s) are also encompassed by the disclosure.

In some embodiments, the provided isolated Tcb1-f nucleic acid(s) encodes a polypeptide comprising an amino acid sequence that is at least 98%, 98.5%, 99%, or 99.5% identical to the amino acid sequence of SEQ ID NO:6 (Tcb1-f). In further embodiments, the encoded polypeptide is at least 98%, 98.5%, 99%, or 99.5% identical to the amino acid sequence of SEQ ID NO:5. In particular embodiments, the encoded polypeptide has at least one Tcb1-f biological activity such as PME activity. Polypeptides encoded by the nucleic acid(s) are also encompassed by the disclosure.

In additional embodiments, the disclosure provides isolated Tcb1-f nucleic acid(s) comprising at least about 10, 15, 20, 25, 30, 35, 40, 45, 50, 75, 100, 125, 150, 175, 200, 250 300, 350, 400, 450, 500, 600, 700, 800, 900, 1,000, 1,100, or 1,200 contiguous nucleotides of the sequence of SEQ ID NO:3 (Tcb1-f, fl na), or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, mat na), or the complete complementary strand thereto.

In additional embodiments, the disclosure provides isolated Tcb1-f nucleic acid(s) comprising a Tcb1-f promoter or fragment or variant thereof. In some embodiments, the nucleic acid comprises at least 100 consecutive nucleotides of the polynucleotide sequence of Tcb1-f promoter. In some embodiments, the nucleic acid comprises at least 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of the sequence of SEQ IDNO:10. In particular embodiments, the Tcb1-f promoter or fragment or variant thereof is able to drive a female specific expression in a plant cell.

In additional embodiments, a Tcb1-f nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the Tcb1-f nucleic acid is operably linked with a female specific promoter. Suitable female specific promoters are described herein and/or otherwise known in the art. In further embodiments, the Tcb1-f nucleic acid is operably linked with a maize female tissue specific promoter. Suitable maze female tissue specific promoters are described herein and/or otherwise known in the art. In yet further embodiments, the Tcb1-f nucleic acid is operably linked with a maize corn silk specific promoter specific promoter.

In some embodiments, the Tcb1-f nucleic acid is operably linked with a polynucleotide sequence comprising a Tcb1-m promoter. In some embodiments, the Tcb1-f nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the Tcb1-f nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the Tcb1-f nucleic acid is operably linked with a polynucleotide sequence comprising a GA1-f promoter. In some embodiments, the Tcb1-f nucleic acid is operably linked with a polynucleotide sequence comprising at least, 75, 100, 125, 150, 175, 200, 225, or 250 contiguous nucleotides of a the sequence of a GA1-f promoter. In some embodiments, the Tcb1-f nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

In some embodiments, the Tcb1-f nucleic acid is operably linked with a polynucleotide sequence comprising a GA2-f promoter. In some embodiments, the Tcb1-f nucleic acid is operably linked with a GA2-f polynucleotide sequence comprising at least, 75, 100, 125, 150, 175, 200, 225, or 250 contiguous nucleotides of a GA2-f promoter.

In some embodiments, the Tcb1-f nucleic acid is operably linked with a polynucleotide sequence comprising a ZmPME10-1 promoter. In some embodiments, the Tcb1-f nucleic acid is operably linked with a ZmPME10-1 polynucleotide sequence comprising at least, 75, 100, 125, 150, 175, 200, 225, or 250 contiguous nucleotides of a ZmPME10-1 promoter.

In other embodiments, the Tcb1-f nucleic acid is operably linked with male specific promoter. Suitable male specific promoters are described herein and/or otherwise known in the art. In further embodiments, the Tcb1-f nucleic acid is operably linked with a pollen/pollen-tube specific promoter. Suitable male pollen/pollen-tube specific promoters are described herein and/or otherwise known in the art. In yet further embodiments, the Tcb1-f nucleic acid is operably linked with a maize pollen/pollen-tube specific promoter. Suitable maize pollen/pollen-tube specific promoters are described herein and/or otherwise known in the art.

In some embodiments, the Tcb1-f nucleic acid is operably linked with a polynucleotide sequence comprising a Tcb1-m promoter. In some embodiments, the Tcb1-f nucleic acid is operably linked with a polynucleotide sequence comprising at least, 75, 100, 125, 150, 175, 200, 225, or 250 contiguous nucleotides of a the sequence of a Tcb1-m promoter. In some embodiments, the Tcb1-f nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the Tcb1-f nucleic acid is operably linked with a polynucleotide sequence comprising a GA1-m promoter. In some embodiments, the Tcb1-f nucleic acid is operably linked with a GA1-m polynucleotide sequence comprising at least, 75, 100, 125, 150, 175, 200, 225, or 250 contiguous nucleotides of a GA1-m promoter.

In some embodiments, the Tcb1-f nucleic acid is operably linked with a polynucleotide sequence comprising a GA2-m promoter. In some embodiments, the Tcb1-f nucleic acid is operably linked with a GA2-m polynucleotide sequence comprising at least, 75, 100, 125, 150, 175, 200, 225, or 250 contiguous nucleotides of a GA2-m promoter.

Expression cassette(s) comprising the Tcb1-f nucleic acid(s) are also provided.

The disclosure further provides vectors comprising the Tcb1-f nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

In additional embodiments, the disclosure provides host cells comprising Tcb1-f nucleic acid(s), expression cassettes and vectors comprising the Tcb1-f nucleic acids provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### ZmPME10-1 Nucleic Acids, Expression Constructs and Vectors

The term “ZmPME10-1 nucleic acid(s)” and grammatical variants thereof, as used herein is intended to encompass ZmPME10-1 nucleic acids provided herein (e.g., polynucleotides encoding the amino acid sequence of SEQ ID NO:21 and SEQ ID NO:22) and fragments and variants thereof. In some embodiments, the disclosure provides nucleic acids that encode ZmPME 10-1 polypeptides. In representative embodiments, the nucleic acids are isolated. In particular embodiments, the ZmPME10-1 fragments or variants have at least one ZmPME10-1 biological activity such as the ability to bind GA1-m having the amino acid sequence of SEQ ID NO:14.

In some embodiments, the disclosure provides an isolated ZmPME10-1 nucleic acid(s) comprising a polynucleotide sequence that encodes the amino acid sequence of SEQ ID NO:21. In further embodiments, the isolated nucleic acid(s) comprises a polynucleotide sequence that encodes the amino acid sequence of SEQ ID NO:22. Polypeptides encoded by the polynucleotides are also encompassed by the disclosure.

In additional embodiments, the disclosure provides an isolated ZmPME10-1 nucleic acid(s) comprising a polynucleotide sequence that encodes a fragment or variant of the amino acid sequence of SEQ ID NO:21. In some embodiments, the polynucleotide sequence encodes a polypeptide comprising a fragment of a protein having the amino acid sequence of at least about 150, 200, 250 300, 350, 400, 450, 500, 550, 600, 650, 655, 660, 665, 670, 657, 676, 677, 678, 679, or 680, contiguous amino acids of SEQ ID NO:21 (ZmPME10-1, mat). In particular embodiments, the encoded polypeptide has at least one ZmPME10-1 biological activity such as the ability to bind GA1-m having the amino acid sequence of SEQ ID NO:14. Polypeptides encoded by the polynucleotides are also encompassed by the disclosure.

In some embodiments, the disclosure provides an isolated ZmPME10 nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1). In some embodiments, the encoded polypeptide comprises an amino acid sequence having a total of one, two, three, four, five, six, or seven, conservative amino acid substitutions from a reference amino acid sequence of SEQ ID NO:21. In some embodiments, the encoded polypeptide comprises an amino acid sequence having a total of one, two, three, four, five, six, or seven, non-conservative amino acid substitutions from a reference amino acid sequence of SEQ ID NO:21. In other embodiments, the encoded polypeptide comprises an amino acid sequence having conservative and non-conservative amino acid substitutions from a reference amino acid sequence of SEQ ID NO:21. In particular embodiments, the encoded polypeptide has at least one ZmPME10-1 biological activity such as the ability to bind GA1-m having the amino acid sequence of SEQ ID NO:14. Polypeptides encoded by the polynucleotides are also encompassed by the disclosure.

In some embodiments, the disclosure provides an isolated ZmPME10 nucleic acid(s) comprising a polynucleotide sequence that encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:21. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:21. In particular embodiments, the encoded polypeptide has at least one ZmPME10-1 biological activity such as the ability to bind GA1-m having the amino acid sequence of SEQ ID NO:14. Polypeptides encoded by the nucleic acid(s) are also encompassed by the disclosure.

In some embodiments, the provided ZmPME10 isolated nucleic acid encodes a polypeptide comprising an amino acid sequence that is at least 80%, 85%, 90%, 95%, 98%, 98.5%, 99%, or 99.5% identical to the amino acid sequence of SEQ ID NO:21 (ZmPME10-1). In further embodiments, the encoded polypeptide is at least 80%, 85%, 90%, 95%, 98%, 98.5%, 99%, or 99.5% identical to the amino acid sequence of SEQ ID NO:22. In particular embodiments, the encoded polypeptide has at least one ZmPME10-1 biological activity such as the ability to bind GA1-m having the amino acid sequence of SEQ ID NO:14. Polypeptides encoded by the nucleic acid(s) are also encompassed by the disclosure.

In additional embodiments, the disclosure provides isolated ZmPME10 nucleic acid(s) comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of a polynucleotide sequence encoding SEQ ID NO:21, or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of a polynucleotide sequence encoding SEQ ID NO:21, or the complete complementary strand thereto.

In additional embodiments, the disclosure provides an isolated nucleic acid(s) comprising a ZmPME10-1 promoter or fragment or variant thereof. In some embodiments, the nucleic acid comprises the polynucleotide sequence of a ZmPME10-1 promoter. In some embodiments, the nucleic acid comprises at least 100 consecutive nucleotides of a ZmPME10-1 promoter.

In additional embodiments, a ZmPME10-1 nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the nucleic acid is operably linked with a female specific promoter. Suitable female specific promoters are described herein and/or otherwise known in the art. In further embodiments, the ZmPME10-1 nucleic acid is operably linked with a maize female tissue specific promoter. Suitable maze female tissue specific promoters are described herein and/or otherwise known in the art. In yet further embodiments, the ZmPME10-1 nucleic acid is operably linked with a maize corn silk specific promoter specific promoter. Suitable maize corn silk specific promoter are described herein and/or otherwise known in the art. In some embodiments, the nucleic acid is operably linked with a ZmPME10-1 promoter sequence.

In some embodiments, the ZmPME10-1 nucleic acid is operably linked with a polynucleotide sequence comprising a ZmPME10-1 promoter. In some embodiments, the Tcb1-m nucleic acid is operably linked with a ZmPME10-1 polynucleotide sequence comprising at least, 75, 100, 125, 150, 175, 200, 225, or 250 contiguous nucleotides of a ZmPME10-1 promoter.

In some embodiments, the ZmPME10-1 nucleic acid is operably linked with a polynucleotide sequence comprising a Tcb1-f promoter. In some embodiments, the Tcb1-m nucleic acid is operably linked with a Tcb1-f promoter polynucleotide sequence comprising at least, 75, 100, 125, 150, 175, 200, 225, or 250 contiguous nucleotides of a Tcb1-f promoter.

In some embodiments, the ZmPME10-1 is operably linked with a polynucleotide sequence comprising a Tcb1-m promoter. In some embodiments, the ZmPME10-1 nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the ZmPME10-1 nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the ZmPME10-1 is operably linked with a polynucleotide sequence comprising a GA1-f promoter. In some embodiments, the ZmPME10-1 nucleic acid is operably linked with a polynucleotide sequence comprising at least 75, 100, 125, 150, 175, 200, 225, or 250 contiguous nucleotides of a GA1-f promoter. In some embodiments, the ZmPME10-1 nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

In some embodiments, the ZmPME10-1 nucleic acid is operably linked with the polynucleotide sequence of a GA2-f promoter. In some embodiments, the nucleic acid is operably linked with a GA2-f polynucleotide sequence comprising at least, 75, 100, 125, 150, 175, 200, 225, or 250 contiguous nucleotides of the sequence of a GA2-f promoter.

In some embodiments, the ZmPME10-1 nucleic acid is operably linked with male specific promoter. Suitable male specific promoters are described herein and/or otherwise known in the art. In further embodiments, the ZmPME10-1 nucleic acid is operably linked with a pollen/pollen-tube specific promoter. Suitable male pollen/pollen-tube specific promoters are described herein and/or otherwise known in the art. In yet further embodiments, the ZmPME10-1 nucleic acid is operably linked with a maize pollen/pollen-tube specific promoter. Suitable maize pollen/pollen-tube specific promoters are described herein and/or otherwise known in the art.

In some embodiments, the ZmPME10-1 nucleic acid is operably linked with a polynucleotide sequence comprising a Tcb1-m promoter. In some embodiments, the ZmPME10-1 nucleic acid is operably linked with a polynucleotide sequence comprising at least, 75, 100, 125, 150, 175, 200, 225, or 250 contiguous nucleotides of a the sequence of a ZmPME10-1 promoter. In some embodiments, the ZmPME10-1 nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the nucleic acid is operably linked with a polynucleotide sequence comprising a GA1-m promoter. In some embodiments, the ZmPME10-1 -m sequence is operably linked with a GA1-m polynucleotide sequence comprising at least 50, 75, 100, 125, 150, 175, 200, 225, or 250 contiguous nucleotides of a the sequence of a GA1-m promoter.

In some embodiments, the nucleic acid is operably linked with a polynucleotide sequence comprising a GA2-m promoter. In some embodiments, the ZmPME10-1 -m sequence is operably linked with a GA2-m polynucleotide sequence comprising at least 50, 75, 100, 125, 150, 175, 200, 225, or 250 contiguous nucleotides of a the sequence of a GA2-m promoter.

Expression cassette(s) comprising the ZmPME10-1 nucleic acid(s) are also provided.

The disclosure further provides vectors comprising the ZmPME10-1 nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

In additional embodiments, the disclosure provides host cells comprising the nucleic acids, expression cassettes and vectors comprising the ZmPME10-1 nucleic acids provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### GA2-m Nucleic Acid(s), Expression Constructs and Vectors

The term “GA2-m nucleic acid(s)” and grammatical variants thereof, as used herein is intended to encompass GA2-m nucleic acids provided herein (e.g., polynucleotides encoding the amino acid sequence of SEQ ID NO:25 (GA2-m, fl) and SEQ ID NO:26 (GA2-m, mat)) and fragments and variants thereof. In some embodiments, the disclosure provides nucleic acids that encode GA2-m polypeptides. In representative embodiments, the nucleic acids are isolated. In particular embodiments, the GA2-m fragments or variants have at least one GA2-m biological activity such as PME activity.

In some embodiments, the disclosure provides an isolated nucleic acid(s) comprising a polynucleotide sequence that encodes the amino acid sequence of SEQ ID NO:26. In some embodiments, the polynucleotide sequence comprises the sequence of SEQ ID NO:28. In further embodiments, the isolated nucleic acid(s) comprises a polynucleotide sequence that encodes the amino acid sequence of SEQ ID NO:25. In yet further embodiments, the polynucleotide sequence comprises the sequence of SEQ ID NO:27. In yet further embodiments, the polynucleotide sequence comprises the sequence of SEQ ID NO:33. Polypeptides encoded by the nucleic acids are also encompassed by the disclosure.

In additional embodiments, the disclosure provides an isolated GA2-m nucleic acid(s) comprising a polynucleotide sequence that encodes a fragment or variant of the amino acid sequence of SEQ ID NO:26. In some embodiments, the polynucleotide sequence encodes a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26. In particular embodiments, the encoded polypeptide has at least one GA2-m biological activity such as PME activity. Polypeptides encoded by the nucleic acids are also encompassed by the disclosure.

In some embodiments, the disclosure provides an isolated GA2-m nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m). In some embodiments, the encoded polypeptide comprises an amino acid sequence having a total of one, two, three, four, five, six, or seven, conservative amino acid substitutions from a reference amino acid sequence of SEQ ID NO:26. In some embodiments, the encoded polypeptide comprises an amino acid sequence having a total of one, two, three, four, five, six, or seven, non-conservative amino acid substitutions from a reference amino acid sequence of SEQ ID NO:26. In other embodiments, the encoded polypeptide comprises an amino acid sequence having conservative and non-conservative amino acid substitutions from a reference amino acid sequence of SEQ ID NO:26. In particular embodiments, the encoded polypeptide has at least one GA2-m biological activity such as PME activity. Polypeptides encoded by the nucleic acids are also encompassed by the disclosure.

In some embodiments, the provided isolated GA2-m nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:26. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:26. In particular embodiments, the encoded polypeptide has at least one GA2-m biological activity such as PME activity. Polypeptides encoded by the nucleic acid(s) are also encompassed by the disclosure.

In some embodiments, the provided GA2-m isolated nucleic acid(s) encodes a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m). In further embodiments, the encoded polypeptide is at least 98%, 98.5%, 99%, or 99.5% identical to the amino acid sequence of SEQ ID NO:25. In particular embodiments, the encoded polypeptide has at least one GA2-m biological activity such as PME activity. Polypeptides encoded by the nucleic acid(s) are also encompassed by the disclosure.

In additional embodiments, the disclosure provides an isolated GA2-m nucleic acid(s) comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na), or the complete complementary strand thereto. In additional embodiments, the disclosure provides an isolated GA2-m nucleic acid(s) comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:33, or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:4 (GA2-m mature na), or the complete complementary strand thereto.

In additional embodiments, a GA2-m nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the GA2-m nucleic acid is operably linked with male specific promoter. Suitable male specific promoters are described herein and/or otherwise known in the art. In further embodiments, the GA2-m nucleic acid is operably linked with a pollen/pollen-tube specific promoter. Suitable male pollen/pollen-tube specific promoters are described herein and/or otherwise known in the art. In yet further embodiments, the GA2-m nucleic acid is operably linked with a maize pollen/pollen-tube specific promoter. Suitable maize pollen/pollen-tube specific promoters are described herein and/or otherwise known in the art.

In some embodiments, the GA2-m nucleic acid is operably linked with a polynucleotide sequence comprising a GA2-m promoter. In some embodiments, the GA2-m nucleic acid is operably linked with a GA2-m polynucleotide sequence comprising at least, 75, 100, 125, 150, 175, 200, 225, or 250 contiguous nucleotides of a the sequence of a GA2-m promoter.

In some embodiments, the GA2-m nucleic acid is operably linked with a polynucleotide sequence comprising a Tcb1-m promoter. In some embodiments, the GA2-m nucleic acid is operably linked with a polynucleotide sequence comprising at least, 75, 100, 125, 150, 175, 200, 225, or 250 contiguous nucleotides of a the sequence of a Tcb1-m promoter. In some embodiments, the GA2-m nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the GA2-m nucleic acid is operably linked with a polynucleotide sequence comprising a GA1-m promoter. In some embodiments, the GA1-m nucleic acid is operably linked with a GA2-m polynucleotide sequence comprising at least, 75, 100, 125, 150, 175, 200, 225, or 250 contiguous nucleotides of a the sequence of a GA1-m promoter.

In some embodiments, the GA2-m nucleic acid is operably linked with a female specific promoter. Suitable female specific promoters are described herein and/or otherwise known in the art. In further embodiments, the GA2-m nucleic acid is operably linked with a maize female tissue specific promoter. Suitable maze female tissue specific promoters are described herein and/or otherwise known in the art. In yet further embodiments, the GA2-m nucleic acid is operably linked with a maize corn silk specific promoter specific promoter. Suitable maize corn silk specific promoter are described herein and/or otherwise known in the art.

In some embodiments, the GA2-m nucleic acid is operably linked with a polynucleotide sequence comprising a GA2-f promoter. In some embodiments, the GA2-m nucleic acid is operably linked with a GA2-f polynucleotide sequence comprising at least, 75, 100, 125, 150, 175, 200, 225, or 250 contiguous nucleotides of a GA2-f promoter.

In some embodiments, the GA2-m nucleic acid is operably linked with a polynucleotide sequence comprising a Tcb1-m promoter. In some embodiments, the GA2-m nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the GA2-m nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the GA2-m nucleic acid is operably linked with a polynucleotide sequence comprising a GA1-f promoter. In some embodiments, the GA2-m nucleic acid is operably linked with a polynucleotide sequence comprising at least, 75, 100, 125, 150, 175, 200, 225, or 250 contiguous nucleotides of a the sequence of a GA2-m promoter. In some embodiments, the GA2-m nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

In some embodiments, the GA2-m nucleic acid is operably linked with a polynucleotide sequence comprising a ZmPME10-1 promoter. In some embodiments, the GA2-m nucleic acid is operably linked with a polynucleotide sequence comprising at least, 75, 100, 125, 150, 175, 200, 225, or 250 contiguous nucleotides of a ZmPME10-1 promoter.

Expression cassette(s) comprising the GA2-m nucleic acid(s) are also provided.

The disclosure further provides vectors comprising the GA2-m nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

The disclosure also provides host cells comprising the GA2-m nucleic acids, expression cassettes and vectors provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### GA2-f Nucleic Acids, Expression Constructs and Vectors

The term “GA2-f nucleic acid(s)” and grammatical variants thereof, as used herein is intended to encompass GA2-f nucleic acids provided herein (e.g., polynucleotides encoding the amino acid sequence of SEQ ID NO:29 (GA2-f, fl) and SEQ ID NO:30 (GA2-f, mat)) and fragments and variants thereof. In additional embodiments, the disclosure provides nucleic acids that encode GA2-f polypeptides. In representative embodiments, the nucleic acids are isolated. In particular embodiments, the GA2-f fragments or variants have at least one GA2-f biological activity such as PME activity.

In some embodiments, the disclosure provides an isolated GA2-f nucleic acid(s) comprising a polynucleotide sequence that encodes the amino acid sequence of SEQ ID NO:30. In some embodiments, the polynucleotide sequence comprises the sequence of SEQ ID NO:32. In further embodiments, the isolated nucleic acid(s) comprises a polynucleotide sequence that encodes the amino acid sequence of SEQ ID NO:29. In yet further embodiments, the polynucleotide sequence comprises the sequence of SEQ ID NO:31. In yet further embodiments, the polynucleotide sequence comprises the sequence of SEQ ID NO:34. Polypeptides encoded by the polynucleotides are also encompassed by the disclosure.

In additional embodiments, the disclosure provides an isolated GA2-f nucleic acid(s) comprising a polynucleotide sequence that encodes a fragment or variant of the amino acid sequence of SEQ ID NO:30. In some embodiments, the polynucleotide sequence encodes a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f, mat). In particular embodiments, the encoded polypeptide has at least one GA2-f biological activity such as PME activity. Polypeptides encoded by the polynucleotides are also encompassed by the disclosure.

In some embodiments, the disclosure provides an isolated GA2-f nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30. In some embodiments, the encoded polypeptide comprises an amino acid sequence having a total of one, two, three, four, five, six, or seven, conservative amino acid substitutions from a reference amino acid sequence of SEQ ID NO:30. In some embodiments, the encoded polypeptide comprises an amino acid sequence having a total of one, two, three, four, five, six, or seven, non-conservative amino acid substitutions from a reference amino acid sequence of SEQ ID NO:30. In other embodiments, the encoded polypeptide comprises an amino acid sequence having conservative and non-conservative amino acid substitutions from a reference amino acid sequence of SEQ ID NO:30. In particular embodiments, the encoded polypeptide has at least one GA2-f biological activity such as PME activity. Polypeptides encoded by the polynucleotides are also encompassed by the disclosure.

In some embodiments, the provided isolated GA2-f nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:30. In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:30. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:30. In particular embodiments, the encoded polypeptide has at least one GA2-f biological activity such as PME. Polypeptides encoded by the nucleic acid(s) are also encompassed by the disclosure.

In some embodiments, the provided isolated GA2-f nucleic acid(s) encodes a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30. In further embodiments, the encoded polypeptide is at least 98%, 98.5%, 99%, or 99.5% identical to the amino acid sequence of SEQ ID NO:29. In particular embodiments, the encoded polypeptide has at least one GA2-m biological activity such as PME activity. Polypeptides encoded by the nucleic acid(s) are also encompassed by the disclosure.

In additional embodiments, the disclosure provides isolated GA2-f nucleic acid(s) comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na), or the complete complementary strand thereto. In further embodiments, the nucleic acid(s) comprise at least about 50, 75, 100, 125, 150, 175, 200, 205, 210, 215, 220, 225, 230, 235, 240, 245, 246, 247, 248, 249, 250, 251, 252, 253, or 254 contiguous nucleotides of the sequence of SEQ ID NO:32 (GA2-f, mat na), or the complete complementary strand thereto. In additional embodiments, the disclosure provides isolated GA2-f nucleic acid(s) comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34, or the complete complementary strand thereto.

In additional embodiments, a GA2-f nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the GA2-f nucleic acid is operably linked with a female specific promoter. Suitable female specific promoters are described herein and/or otherwise known in the art. In further embodiments, the GA2-f nucleic acid is operably linked with a maize female tissue specific promoter. Suitable maze female tissue specific promoters are described herein and/or otherwise known in the art. In yet further embodiments, the GA2-f nucleic acid is operably linked with a maize corn silk specific promoter specific promoter. Suitable maize corn silk specific promoter are described herein and/or otherwise known in the art.

In some embodiments, the GA2-f nucleic acid is operably linked with a polynucleotide sequence comprising a GA2-f promoter. In some embodiments, the GA2-f nucleic acid is operably linked with a GA2-f polynucleotide sequence comprising at least, 75, 100, 125, 150, 175, 200, 225, or 250 contiguous nucleotides of a the sequence of a GA2-f promoter.

In some embodiments, the GA2-f nucleic acid is operably linked with a polynucleotide sequence comprising a Tcb1-f promoter. In some embodiments, the GA2-f nucleic acid is operably linked with a polynucleotide sequence comprising at least, 75, 100, 125, 150, 175, 200, 225, or 250 contiguous nucleotides of a the sequence of a Tcb1-f promoter.

In some embodiments, the GA2-f nucleic acid is operably linked with a polynucleotide sequence comprising a Tcb1-m promoter. In some embodiments, the GA2-f nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the GA2-f nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the GA2-f nucleic acid is operably linked with a polynucleotide sequence comprising a GA1-f promoter. In some embodiments, the GA2-f nucleic acid is operably linked with a polynucleotide sequence comprising at least, 75, 100, 125, 150, 175, 200, 225, or 250 contiguous nucleotides of a the sequence of a GA1-f promoter. In some embodiments, the GA2-f nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

In some embodiments, the GA2-f nucleic acid is operably linked with a polynucleotide sequence comprising a Tcb1-f promoter. In some embodiments, the GA2-f nucleic acid is operably linked with a polynucleotide sequence comprising at least, 75, 100, 125, 150, 175, 200, 225, or 250 contiguous nucleotides of a the sequence of a Tcb1-f promoter.

In some embodiments, the GA2-f nucleic acid is operably linked with a polynucleotide sequence comprising a ZmPME10-1 promoter. In some embodiments, the GA2-f nucleic acid is operably linked with a polynucleotide sequence comprising at least, 75, 100, 125, 150, 175, 200, 225, or 250 contiguous nucleotides of a ZmPME10-1 promoter.

In other embodiments, the GA2-f nucleic acid is operably linked with male specific promoter. Suitable male specific promoters are described herein and/or otherwise known in the art. In further embodiments, the GA2-f nucleic acid is operably linked with a pollen/pollen-tube specific promoter. Suitable male pollen/pollen-tube specific promoters are described herein and/or otherwise known in the art. In yet further embodiments, the GA2-f nucleic acid is operably linked with a maize pollen/pollen-tube specific promoter. Suitable maize pollen/pollen-tube specific promoters are described herein and/or otherwise known in the art.

In some embodiments, the GA2-f nucleic acid is operably linked with a polynucleotide sequence comprising a GA2-m promoter. In some embodiments, the GA2-f nucleic acid is operably linked with a GA2-m polynucleotide sequence comprising at least, 75, 100, 125, 150, 175, 200, 225, or 250 contiguous nucleotides of a GA2-m promoter.

In some embodiments, the GA2-f nucleic acid is operably linked with a polynucleotide sequence comprising a Tcb1-m promoter. In some embodiments, the GA2-f nucleic acid is operably linked with a polynucleotide sequence comprising at least, 75, 100, 125, 150, 175, 200, 225, or 250 contiguous nucleotides of a the sequence of a Tcb1-m promoter. In some embodiments, the GA2-f nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the GA2-f nucleic acid is operably linked with a polynucleotide sequence comprising a GA1-m promoter. In some embodiments, the GA2-m nucleic acid is operably linked with a GA1-m polynucleotide sequence comprising at least, 75, 100, 125, 150, 175, 200, 225, or 250 contiguous nucleotides of a GA1-m promoter.

Expression cassette(s) comprising the GA2-f nucleic acid(s) are also provided.

The disclosure further provides vectors comprising the GA2-f nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

The disclosure also provides host cells comprising the nucleic acids, expression cassettes and vectors provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### GA1-m Nucleic Acids, Expression Constructs and Vectors

The term “GA1-m nucleic acid(s)” and grammatical variants thereof, as used herein is intended to encompass GA1-m nucleic acids specifically described herein (e.g., polynucleotides encoding the amino acid sequence of SEQ ID NO:13 and SEQ ID NO:14) and fragments and variants thereof. In some embodiments, the disclosure provides nucleic acids that encode GA1-m polypeptides. In representative embodiments, the nucleic acids are isolated. In particular embodiments, the GA1-m fragments or variants have at least one GA1-m biological activity such as PME activity or the ability to bind ZmPME10-1 having the amino acid sequence of SEQ ID NO:21.

In some embodiments, the disclosure provides an isolated GA1-m nucleic acid(s) comprising a polynucleotide sequence that encodes the amino acid sequence of SEQ ID NO:14. In some embodiments, the polynucleotide sequence comprises the sequence of SEQ ID NO:16. In further embodiments, the isolated nucleic acid(s) comprises a polynucleotide sequence that encodes the amino acid sequence of SEQ ID NO:13. In yet further embodiments, the polynucleotide sequence comprises the sequence of SEQ ID NO:15. Polypeptides encoded by the polynucleotides are also encompassed by the disclosure.

In additional embodiments, the disclosure provides an isolated GA1-m nucleic acid(s) comprising a polynucleotide sequence that encodes a fragment or variant of the amino acid sequence of SEQ ID NO: 14. In some embodiments, the polynucleotide sequence encodes a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, or 354, contiguous amino acids of SEQ ID NO:14 (GA1-m, mat). In particular embodiments, the encoded polypeptide has at least one GA1-m biological activity such as PME activity or the ability to bind ZmPME10-1 having the amino acid sequence of SEQ ID NO:21. Polypeptides encoded by the polynucleotides are also encompassed by the disclosure.

In some embodiments, the disclosure provides an isolated GA1-m nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:14 (GA1-m). In some embodiments, the encoded polypeptide comprises an amino acid sequence having a total of one, two, three, four, five, six, or seven, conservative amino acid substitutions from a reference amino acid sequence of SEQ ID NO:14. In some embodiments, the encoded polypeptide comprises an amino acid sequence having a total of one, two, three, four, five, six, or seven, non-conservative amino acid substitutions from a reference amino acid sequence of SEQ ID NO:14. In other embodiments, the encoded polypeptide comprises an amino acid sequence having conservative and non-conservative amino acid substitutions from a reference amino acid sequence of SEQ ID NO:14. In particular embodiments, the encoded polypeptide has at least one GA1-m biological activity such as PME activity or the ability to bind ZmPME10-1 having the amino acid sequence of SEQ ID NO: 21. Polypeptides encoded by the polynucleotides are also encompassed by the disclosure.

In some embodiments, the isolated GA1-m nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:14 (GA1-m). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:14. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:14. In particular embodiments, the encoded polypeptide has at least one GA1-m biological activity such as PME activity or the ability to bind ZmPME10-1 having the amino acid sequence of SEQ ID NO:21. Polypeptides encoded by the nucleic acid(s) are also encompassed by the disclosure.

In some embodiments, the disclosure provides an isolated GA1-m nucleic acid(s) nucleic acid(s) encodes a polypeptide comprising an amino acid sequence that is at least 98%, 98.5%, 99%, or 99.5% identical to the amino acid sequence of SEQ ID NO:14 (GA1-m). In further embodiments, the encoded polypeptide is at least 98%, 98.5%, 99%, or 99.5% identical to the amino acid sequence of SEQ ID NO:13. In particular embodiments, the encoded polypeptide has at least one GA1-m biological activity such as PME activity or the ability to bind ZmPME10-1 having the amino acid sequence of SEQ ID NO:21. Polypeptides encoded by the nucleic acid(s) are also encompassed by the disclosure.

In additional embodiments, the disclosure provides an isolated GA1-m nucleic acid(s) comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 (GA1-m, fl na), or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:16 (GA1-m mature na), or the complete complementary strand thereto.

In additional embodiments, the disclosure provides an isolated nucleic acid(s) comprising a GA1-m promoter or fragment or variant thereof. In some embodiments, the nucleic acid comprises the polynucleotide sequence of a GA1-m promoter. In some embodiments, the nucleic acid comprises at least 100 consecutive nucleotides of the polynucleotide sequence of a GA1-m promoter. In other embodiments, the nucleic acid comprises a polynucleotide sequence that hybridizes to the complete complement of the polynucleotide sequence of a GA1-m promoter or the polynucleotide sequence of 50, 75, 100, or 150, consecutive nucleotides of the polynucleotide sequence of a GA1-m promoter under stringent hybridization conditions. In another embodiment, the isolated nucleic acid(s) comprises a GA1-m promoter or fragment thereof having at least 95% sequence identity to the polynucleotide sequence of a GA1-m promoter or at least 100 consecutive nucleotides of the polynucleotide sequence of a GA1-m promoter. In particular embodiments, the GA1-m promoter or fragment or variant thereof is able to drive male specific expression in a plant cell.

In additional embodiments, a GA1-m nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the nucleic acid is operably linked with male specific promoter. Suitable male specific promoters are described herein and/or otherwise known in the art. In further embodiments, the nucleic acid is operably linked with a pollen/pollen-tube specific promoter. Suitable male pollen/pollen-tube specific promoters are described herein and/or otherwise known in the art. In yet further embodiments, the nucleic acid is operably linked with a maize pollen/pollen-tube specific promoter. Suitable maize pollen/pollen-tube specific promoters are described herein and/or otherwise known in the art.

In some embodiments, the nucleic acid is operably linked with a polynucleotide sequence comprising a GA2-m promoter. In some embodiments, the nucleic acid is operably linked with a GA2-m polynucleotide sequence comprising at least, 75, 100, 125, 150, 175, 200, 225, or 250 contiguous nucleotides of a GA2-m promoter.

In some embodiments, the nucleic acid is operably linked with a polynucleotide sequence comprising a Tcb1-m promoter. In some embodiments, the nucleic acid is operably linked with a polynucleotide sequence comprising at least, 75, 100, 125, 150, 175, 200, 225, or 250 contiguous nucleotides of a the sequence of a Tcb1-m promoter. In some embodiments, the nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. In particular embodiments, the Tcb1-m promoter or fragment or variant thereof is able to drive male tissue specific expression in a plant cell.

In some embodiments, the nucleic acid is operably linked with a polynucleotide sequence comprising a ZmPME10-1 -m promoter. In some embodiments, the nucleic acid is operably linked with a GA2-m polynucleotide sequence comprising at least, 75, 100, 125, 150, 175, 200, 225, or 250 contiguous nucleotides of a ZmPME10-1 promoter.

In some embodiments, the GA1-m nucleic acid is operably linked with a female specific promoter. Suitable female specific promoters are described herein and/or otherwise known in the art. In further embodiments, the GA1-m nucleic acid is operably linked with a maize female tissue specific promoter. Suitable maze female tissue specific promoters are described herein and/or otherwise known in the art. In yet further embodiments, the GA1-m nucleic acid is operably linked with a maize corn silk specific promoter specific promoter. Suitable maize corn silk specific promoter are described herein and/or otherwise known in the art.

In some embodiments, the GA1-m nucleic acid is operably linked with a polynucleotide sequence comprising a Tcb1-m promoter. In some embodiments, the GA1-m nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the GA1-m nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the GA1-m nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

Expression cassette(s) comprising the GA1-m nucleic acid(s) are also provided.

The disclosure further provides vectors comprising the GA1-m nucleic acid(s) and expression cassette(s) provided herein, including expression vectors, transformation vectors and vectors for replicating and/or manipulating the polynucleotide sequences in plants and other organisms (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC, YAC, and/or a cosmid.

In additional embodiments, the disclosure provides host cells comprising the nucleic acids, expression cassettes and vectors comprising the GA1-m nucleic acids provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### GA1-f Nucleic Acids, Expression Constructs and Vectors

The term “GA1-f nucleic acid(s)” and grammatical variants thereof, as used herein is intended to encompass GA1-f nucleic acids specifically described herein (e.g., polynucleotides encoding the amino acid sequence of SEQ ID NO:17 and SEQ ID NO:18) and fragments and variants thereof. In additional embodiments, the disclosure provides nucleic acids that encode GA1-f polypeptides. In representative embodiments, the nucleic acids are isolated. In particular embodiments, the GA1-f fragments or variants have at least one GA1-f biological activity such as PME activity.

In some embodiments, the disclosure provides an isolated GA1-f nucleic acid(s) comprising a polynucleotide sequence that encodes the amino acid sequence of SEQ ID NO:18. In some embodiments, the polynucleotide sequence comprises the sequence of SEQ ID NO:20. In further embodiments, the isolated nucleic acid(s) comprises a polynucleotide sequence that encodes the amino acid sequence of SEQ ID NO:17. In yet further embodiments, the polynucleotide sequence comprises the sequence of SEQ ID NO:20. Polypeptides encoded by the polynucleotides are also encompassed by the disclosure.

In additional embodiments, the disclosure provides an isolated GA1-f nucleic acid(s) comprising a polynucleotide sequence that encodes a fragment or variant of the amino acid sequence of SEQ ID NO:18. In some embodiments, the polynucleotide sequence encodes a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18 (GA1-f mature). In particular embodiments, the encoded polypeptide has at least one GA1-f biological activity such as PME activity. Polypeptides encoded by the polynucleotides are also encompassed by the disclosure.

In some embodiments, the disclosure provides an isolated GA1-f nucleic acid(s) comprising a polynucleotide sequence that encodes a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:18 (GA1-f). In some embodiments, the encoded polypeptide comprises an amino acid sequence having a total of one, two, three, four, five, six, or seven, conservative amino acid substitutions from a reference amino acid sequence of SEQ ID NO:18. In some embodiments, the encoded polypeptide comprises an amino acid sequence having a total of one, two, three, four, five, six, or seven, non-conservative amino acid substitutions from a reference amino acid sequence of SEQ ID NO:18. In other embodiments, the encoded polypeptide comprises an amino acid sequence having conservative and non-conservative amino acid substitutions from a reference amino acid sequence of SEQ ID NO:18. In particular embodiments, the encoded polypeptide has at least one GA1-f biological activity such as PME activity. Polypeptides encoded by the polynucleotides are also encompassed by the disclosure.

In some embodiments, the provided isolated GA1-f nucleic acid(s) encodes a polypeptide comprising an amino acid sequence having fewer than eight, fewer than seven, fewer than six, fewer than five, fewer than four, fewer than three, or fewer than two amino acid substitutions, deletions, and/or insertions from a reference amino acid sequence of SEQ ID NO:18 (GA1-f). In some embodiments, the encoded polypeptide comprises an amino acid sequence having fewer than eight, fewer than seven, fewer than six, fewer than five, fewer than four, fewer than three, or fewer than two, conservative amino acid substitutions from a reference amino acid sequence of SEQ ID NO:18. In some embodiments, the encoded polypeptide comprises an amino acid sequence having fewer than eight, fewer than seven, fewer than six, fewer than five, fewer than four, fewer than three, or fewer than two non-conservative amino acid substitutions from a reference amino acid sequence of SEQ ID NO:18. In particular embodiments, the encoded polypeptide has at least one GA1-f biological activity such as PME. Polypeptides encoded by the nucleic acid(s) are also encompassed by the disclosure.

In some embodiments, the provided isolated GA1-f nucleic acid(s) encodes a polypeptide comprising an amino acid sequence that is at least 98%, 98.5%, 99%, or 99.5% identical to the amino acid sequence of SEQ ID NO:18 (GA1-f). In further embodiments, the encoded polypeptide is at least 98%, 98.5%, 99%, or 99.5% identical to the amino acid sequence of SEQ ID NO:17. In particular embodiments, the encoded polypeptide has at least one Tcb1-m biological activity such as PME activity. Polypeptides encoded by the nucleic acid(s) are also encompassed by the disclosure.

In additional embodiments, the disclosure provides isolated GA1-f nucleic acid(s) comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:20 (GA1-f, fl na), or the complete complementary strand thereto. In further embodiments, the nucleic acid(s) comprise at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:20 (GA1-f, mat na), or the complete complementary strand thereto.

In additional embodiments, the disclosure provides isolated GA1-f nucleic acid(s) comprising a GA1-f promoter or fragment or variant thereof. In some embodiments, the nucleic acid comprises the polynucleotide sequence of SEQ ID NO:12 (GA1-f, prom). In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive male specific expression in a plant cell.

In additional embodiments, a GA1-f nucleic acid provided herein is operably linked with a promoter. In some embodiments, the nucleic acid is operably linked with a constitutive promoter. Suitable constitutive promoters are described herein and/or otherwise known in the art. In other embodiments, the nucleic acid is operably linked with an inducible promoter. Suitable inducible promoters are described herein and/or otherwise known in the art. In some embodiments, the nucleic acid is operably linked with a heterologous promoter. In some embodiments, the nucleic acid is operably linked with an endogenous promoter.

In some embodiments, the GA1-f nucleic acid is operably linked with a female specific promoter. Suitable female specific promoters are described herein and/or otherwise known in the art. In further embodiments, the GA1-f nucleic acid is operably linked with a maize female tissue specific promoter. Suitable maze female tissue specific promoters are described herein and/or otherwise known in the art. In yet further embodiments, the GA1-f nucleic acid is operably linked with a maize corn silk specific promoter specific promoter. Suitable maize corn silk specific promoter are described herein and/or otherwise known in the art.

In some embodiments, the Tcb1-f nucleic acid is operably linked with a polynucleotide sequence comprising a GA1-f promoter. In some embodiments, the GA1-f nucleic acid is operably linked with a polynucleotide sequence comprising at least, 75, 100, 125, 150, 175, 200, 225, 250, 300, 350, 375, 400, 450, 500, 750, 1,000, 1,250, 2,000, 2,500, 3,000, 4,000, or 5,000 contiguous nucleotides of a the sequence of a Tcb1-f promoter. In some embodiments, the GA1-f nucleic acid is operably linked with a Tcb1-m polynucleotide sequence comprising the nucleic acid sequence of SEQ ID NO: 10, or a fragment thereof. In some embodiments, the nucleic acid comprises at least 100 consecutive nucleotides of the polynucleotide sequence of SEQ ID NO:10. In other embodiments, the nucleic acid comprises a polynucleotide sequence that hybridizes to the complete complement of the polynucleotide sequence of SEQ ID NO:10 or the polynucleotide sequence of 50, 75, 100, or 150, consecutive nucleotides of the polynucleotide sequence of SEQ ID NO:10 under stringent hybridization conditions. In another embodiment, the isolated nucleic acid(s) comprises a Tcb1-f promoter or fragment thereof having at least 95% sequence identity to the polynucleotide sequence of SEQ ID NO:10 or at least 100 consecutive nucleotides of the polynucleotide sequence of SEQ ID NO:10. In particular embodiments, the Tcb1-f promoter or fragment or variant thereof is able to drive female tissue specific expression in a plant cell.

In some embodiments, the GA1-f nucleic acid is operably linked with a GA1-f polynucleotide sequence comprising the nucleic acid sequence of SEQ ID NO: 12, or a fragment thereof is operably linked with a polynucleotide sequence comprising a GA1-f promoter. In some embodiments, the GA2-f nucleic acid is operably linked with a polynucleotide sequence comprising at least, 75, 100, 125, 150, 175, 200, 225, or 250 contiguous nucleotides of a the sequence of a GA1-f promoter. In some embodiments, the GA2-f nucleic acid is operably linked with a GA1-f polynucleotide sequence comprising the nucleic acid sequence of SEQ ID NO: 12, or a fragment thereof. In some embodiments, the nucleic acid comprises at least 100 consecutive nucleotides of the polynucleotide sequence of SEQ ID NO:12. In other embodiments, the nucleic acid comprises a polynucleotide sequence that hybridizes to the complete complement of the polynucleotide sequence of SEQ ID NO:12 or the polynucleotide sequence of 50, 75, 100, or 150, consecutive nucleotides of the polynucleotide sequence of SEQ ID NO:12 under stringent hybridization conditions. In another embodiment, the isolated nucleic acid(s) comprises a GA1-f promoter or fragment thereof having at least 95% sequence identity to the polynucleotide sequence of SEQ ID NO:12 or at least 100 consecutive nucleotides of the polynucleotide sequence of SEQ ID NO:12. In particular embodiments, the GA1-f promoter or fragment or variant thereof is able to drive female tissue specific expression in a plant cell.

In some embodiments, the GA1-f nucleic acid is operably linked with a GA1-f polynucleotide sequence comprising the nucleic acid sequence of SEQ ID NO: 12, or a fragment thereof. In some embodiments, the nucleic acid is operably linked with a GA1-f polynucleotide sequence comprising at least, 75, 100, 125, 150, 175, 200, 225, or 250 contiguous nucleotides of the sequence of SEQ ID NO: 12. In other embodiments, the nucleic acid is operably linked with a nucleic acid sequence that hybridizes with the polynucleotide sequence of SEQ ID NO: 12, or the reverse complementary sequence thereof, under stringent hybridization conditions.

In other embodiments, the GA1-f nucleic acid is operably linked with male specific promoter. Suitable male specific promoters are described herein and/or otherwise known in the art. In further embodiments, the GA1-f nucleic acid is operably linked with a pollen/pollen-tube specific promoter. Suitable male pollen/pollen-tube specific promoters are described herein and/or otherwise known in the art. In yet further embodiments, the GA1-f nucleic acid is operably linked with a maize pollen/pollen-tube specific promoter. Suitable maize pollen/pollen-tube specific promoters are described herein and/or otherwise known in the art.

In some embodiments, the GA1-f nucleic acid is operably linked with a polynucleotide sequence comprising a GA1-m promoter. In some embodiments, the GA1-f nucleic acid is operably linked with a polynucleotide sequence comprising at least, 75, 100, 125, 150, 175, 200, 225, or 250 contiguous nucleotides of a GA1-m promoter.

In some embodiments, the GA1-f nucleic acid is operably linked with a polynucleotide sequence comprising a Tcb1-m promoter. In some embodiments, the GA1-f nucleic acid is operably linked with a polynucleotide sequence comprising at least, 75, 100, 125, 150, 175, 200, 225, or 250 contiguous nucleotides of a the sequence of a Tcb1-m promoter. In some embodiments, the GA2-f nucleic acid is operably linked with a Tcb1-m polynucleotide sequence comprising the nucleic acid sequence of SEQ ID NO: 9, or a fragment thereof. In some embodiments, the nucleic acid comprises at least 100 consecutive nucleotides of the polynucleotide sequence of SEQ ID NO:9. In other embodiments, the nucleic acid comprises a polynucleotide sequence that hybridizes to the complete complement of the polynucleotide sequence of SEQ ID NO:9 or the polynucleotide sequence of 50, 75, 100, or 150, consecutive nucleotides of the polynucleotide sequence of SEQ ID NO:9 under stringent hybridization conditions. In another embodiment, the isolated nucleic acid(s) comprises a Tcb1-m promoter or fragment thereof having at least 95% sequence identity to the polynucleotide sequence of SEQ ID NO:9 or at least 100 consecutive nucleotides of the polynucleotide sequence of SEQ ID NO:9. Tcb1-m polynucleotide sequence comprising the nucleic acid sequence of SEQ ID NO: 9, or a fragment thereof. In some embodiments, the GA1-f nucleic acid is operably linked with a Tcb1-m polynucleotide sequence comprising at least, 75, 100, 125, 150, 175, 200, 225, or 250 contiguous nucleotides of the sequence of SEQ ID NO: 9. In other embodiments, the GA1-f nucleic acid is operably linked with a nucleic acid sequence that hybridizes with the polynucleotide sequence of SEQ ID NO: 9, or the reverse complementary sequence thereof, under stringent hybridization conditions.

In some embodiments, the GA1-f nucleic acid is operably linked with a polynucleotide sequence comprising a GA1-m promoter. In some embodiments, the GA1-f nucleic acid is operably linked with a polynucleotide sequence comprising at least, 75, 100, 125, 150, 175, 200, 225, or 250 contiguous nucleotides of a GA1-m promoter.

Expression cassette(s) comprising the GA1-f nucleic acid(s) are also provided.

The disclosure further provides vectors comprising the GA1-f nucleic acid(s) and expression cassette(s) provided herein, including expression vectors, transformation vectors and vectors for replicating and/or manipulating the polynucleotide sequences in organisms other than plants (e.g., a bacteria or fungi such as yeast). The vector can be a plant vector, animal (e.g., insect or mammalian) vector, bacterial vector, yeast vector or fungal vector. Generally, the vector is a plant vector, a bacterial vector, or a shuttle vector that can replicate in either host under appropriate conditions. Bacterial and plant vectors are well-known in the art. Exemplary plant vectors include plasmids (e.g., pUC or the Ti plasmid), cosmids, phage, bacterial artificial chromosomes (BACs), yeast artificial chromosomes (YACs) and plant viruses. In particular embodiments, the vector is not a BAC ora YAC.

In additional embodiments, the disclosure provides host cells comprising the nucleic acids, expression cassettes and vectors comprising the GA1-f nucleic acids provided herein. The host cell can be transiently or stably transformed with the nucleic acid, expression cassette or vector. Further, the host cell can be a cultured cell, a cell obtained from a plant, plant part, or plant tissue, or a cell in situ in a plant, plant part or plant tissue. The host cells can be from any suitable species, including plant (e.g., maize), bacterial, yeast, insect and/or mammalian cells. In representative embodiments, the cell is a plant cell or bacterial cell. In particular embodiments, the host cell is a plant cell. In some embodiments, the plant cell is a monocot cell. In further embodiments, the host cell is a maize cell, wheat cell, rice cell, barley cell, oat cell, millet cell, rye cell, turfgrass cell, fescue cell, sorghum cell, or a sugarcane cell. In particular embodiments, the host cell is a maize cell. In particular embodiments, the host cell is an algal cell. In some embodiments, the plant cell is a dicot cell. In further embodiments, the host cell is a soybean cell, canola cell, sunflower cell, sugar beet cell, quinoa cell, alfalfa cell, or a cotton cell.

### Methods of Introducing Nucleic Acids

In some embodiments, the disclosure provides transgenic plants, plant parts and plant cells comprising the nucleic acids, expression cassettes, and/or vectors provided herein.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and Agrobacterium, physicochemical methods such as electroporation (see, e.g., Fromm et al., PNAS 82:5824 (1985)), protoplast fusion (see, e.g., Fraley et al., PNAS 79:1859 (1982)), floral dip methods, polyethylene glycol (see, e.g., Krens et al., Nature 296:72 (1982))., ballistic bombardment (see, e.g., U.S. Pat. Nos. 5,015,580 (soybean); 5,914,451 (soybean); 5,550,318 (corn); 5,538,880 (corn); 6,160,208 (corn); 6,399,861 (corn); 6,153,812 (wheat) and 6,365,807 (rice)), microinjection, and the like. Other transformation technology includes the whiskers technology (see, e.g., U.S. Pat. Nos. 5,302,523 and 5,464,765) and pollen tube transformation. In one form of direct transformation, the vector is microinjected directly into plant cells by use of micropipettes to mechanically transfer the recombinant DNA (see, e.g., Crossway, Mol. Gen. Genetics 202:179 (1985)).

In some embodiments, the plants are transformed with nucleic acid(s) provided herein using an Agrobacterium-mediated nucleic acid transfer. Agrobacterium-mediated nucleic acid transfer exploits the natural ability of A. tumefaciens and A. rhizogenes to transfer DNA into plant chromosomes. Transfer by means of engineered Agrobacterium strains has become routine for many dicotyledonous plants and has been achieved in several monocot species, including cereal species such as maize (Rhodes et al., Science 240, 204 (1988)), rice (Hiei et al., Plant J. 6:271 (1994)), and rye. Exemplary Agrobacterium-mediated transformation methods are described, for example, in U.S. Pat. Nos. 5,591,616 (corn); 7,026,528 (wheat) and 6,329,571 (rice), 5,159,135 (cotton); 5,824,877 (soybean); 5,463,174 (canola); 5,846,797 (cotton); 8,044,260 (cotton); 6,384,301 (soybean), U.S. Publ. No. 2004/0087030 (cotton), and U.S. Publ. No. 2001/0042257 (sugar beet), all of which are incorporated herein by reference in their entirety. Plant host cells can be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

In another embodiment, the pollination barrier factor nucleic acid is transformed into a plant cell via genome editing. In a further embodiment, the transformed pollination barrier factor nucleic acid encodes a Tcb1-f, Tcb1-m, GA1-f, GA1-m GA2-f, GA2-m, and/or ZmPME10-1 provided herein and the encoded polypeptide has at least one biological activity (e.g., PME). In some embodiments, the pollination barrier factor nucleic acid is transformed into a plant cell via genome editing using for example a recombinant DNA donor template at a predetermined site of the genome by methods of site-directed integration. Site-directed integration may be accomplished by any method known in the art, including, but not limited to zinc-finger nucleases, engineered or native meganucleases, TALE-endonucleases, or an RNA-guided endonuclease (for example Cas9 or Cpf1). The recombinant DNA construct may be inserted at the pre-determined site by homologous recombination (HR) or by nonhomologous end joining (NHEJ). In addition to insertion of a recombinant DNA construct into a plant chromosome at a pre-determined site, genome editing can be achieved through oligonucleotide-directed mutagenesis (ODM) (U.S. Pat. No. 8,268,622) or by introduction of a double-strand break (DSB) or nick with a site specific nuclease, followed by NHEJ or repair. The repair of the DSB or nick may be used to introduce insertions or deletions at the site of the DSB or nick, and these mutations may result in the introduction of frame-shifts, amino acid substitutions, and/or an early termination codon of protein translation or alteration of a regulatory sequence of a gene. Genome editing may be achieved with or without a donor template molecule.

Protoplasts, which have been transformed by any method known in the art, can also be regenerated to produce intact plants using known techniques.

Plant cells or tissues, including protoplasts, which have been transformed by any method known in the art, can be regenerated to produce intact plants using known techniques. (see, e.g., Evans et al., Handbook of Plant Cell Cultures, Vol. 1: (MacMillan Publishing Co. New York, 1983); and Vasil I. R. (ed.), Cell Culture and Somatic Cell Genetics of Plants, Acad. Press, Orlando, Vol. I, 1984, and Vol. II, 1986). Means for regeneration vary from species to species of plants, but generally a suspension of transformed protoplasts or a petri plate containing transformed explants is first provided. Transformation of plant material can be practiced in tissue culture on nutrient media in vitro. Recipient cell targets include, but are not limited to, meristem cells, shoot tips, hypocotyls, calli, immature or mature embryos, and gametic cells such as microspores, pollen, sperm and egg cells. Cells containing a transgenic nucleus are grown into transgenic plants. Callus tissue is formed and shoots can be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. The regenerated plants are transferred to standard soil conditions and cultivated in a conventional manner. The plants are grown and harvested using conventional procedures.

In addition to direct transformation of a plant material with a recombinant DNA construct, a transgenic plant can be prepared by crossing a first plant comprising a recombinant DNA with a second plant lacking the recombinant DNA. For example, recombinant DNA can be introduced into a first plant line that is amenable to transformation, which can be crossed with a second plant line to introgress the recombinant DNA into the second plant line.

In particular embodiments, the introgression of Tcb1-m nucleic acid(s) and/or Tcb1-f nucleic acid(s) into a plant or plant cell (e.g., an inbred, hybrid, haploid, apomictic and/or genetically engineered plant or plant cell) which does not contain one or both of Tcb1-m and/or Tcb1-f traits produces a new cross-incompatible, cross-compatible, or self-incompatible plant/cell containing the Tcb1-m nucleic acid(s) and/or Tcb1-f nucleic acid(s). In particular embodiments, the plant or plant cell is maize, or a maize cell, respectively.

In particular embodiments, the introgression of GA2-m nucleic acid(s) and/or GA2-f nucleic acid(s) into a plant or plant cell (e.g., an inbred, hybrid, haploid, apomictic and/or genetically engineered plant or plant cell) which does not contain one or both of GA2-m and/or GA2-f traits produces a new cross-incompatible, cross-compatible, or self-incompatible plant/cell containing the GA2-m nucleic acid(s) and/or GA2-f nucleic acid(s). In particular embodiments, the plant or plant cell is maize, or a maize cell, respectively.

In some embodiments, the disclosure provides a method of introducing a nucleic acid, expression cassette or vector provided herein into a plant, plant part or plant cell. In representative embodiments, the method comprises transforming the plant, plant part or plant cell with a Tcb1-m nucleic acid, expression cassette, or vector provided herein. In some embodiments, the Tcb1-m nucleic acid is operably associated with a polynucleotide sequence of interest (e.g., a heterologous polynucleotide sequence of interest). In some embodiments, the polynucleotide sequence of interest is a promoter sequence. In further embodiments, the promoter sequence is a constitutive or inducible promoter sequence. In other embodiments, the polynucleotide sequence of interest is a male- or female-specific promoter sequence described herein or otherwise known in the art. In other embodiments, the Tcb1-m nucleic acid comprises a Tcb1-m promoter sequence, or fragment thereof, and the polynucleotide sequence of interest encodes a polypeptide that imparts a desirable agronomic trait to the plant (e.g., drought resistance, heat resistance, salt resistance, disease resistance, insect and other pest resistance [e.g., a Bacillus thuringiensis endotoxin], herbicide resistance, and the like), confers male sterility, improves fertility and/or improves nutritional quality. The disclosure further comprises host plants, cells, plant parts, seeds, tissue culture (including callus) transiently or stably transformed with the Tcb1-m nucleic acids, expression cassettes or vectors provided herein.

In some embodiments, the method comprises transforming the plant, plant part or plant cell with a Tcb1-f nucleic acid, expression cassette, or vector provided herein. In some embodiments, the Tcb1-f nucleic acid is operably associated with a polynucleotide sequence of interest (e.g., a heterologous polynucleotide sequence of interest). In some embodiments, the polynucleotide sequence of interest is a promoter sequence. In further embodiments, the promoter sequence is a constitutive or inducible promoter sequence. In other embodiments, the polynucleotide sequence of interest is a male- or female-specific promoter sequence described herein or otherwise known in the art. In other embodiments, the Tcb1-f nucleic acid comprises a Tcb1-f promoter sequence, or fragment thereof, and the polynucleotide sequence of interest encodes a polypeptide that imparts a desirable agronomic trait to the plant (e.g., resistance to an abiotic stress such as drought, heat, and salt; disease resistance, insect and other pest resistance [e.g., a Bacillus thuringiensis endotoxin], herbicide resistance, and the like), confers male sterility, improves fertility and/or improves nutritional quality. The disclosure further comprises host plants, cells, plant parts, seeds, tissue culture (including callus) transiently or stably transformed with the Tcb1-f nucleic acids, expression cassettes or vectors provided herein.

In some embodiments, the disclosure provides a transgenic plant comprising Tcb1-f nucleic acid(s) and/or Tcb1-m nucleic acid(s), and expression cassette(s), and/or vector(s) provided herein. The plant can be transiently or stably transformed with the nucleic acid(s), expression cassette(s) or vector(s). In representative embodiments, the plant comprises a cell or plant part provided herein. In further embodiments, the transgenic plant has a cross-incompatible, cross-compatible, or self-incompatible phenotype.

In some embodiments, the disclosure provides a method of introducing a nucleic acid, expression cassette or vector provided herein into a plant, plant part or plant cell. In representative embodiments, the method comprises transforming the plant, plant part or plant cell with a GA1-m nucleic acid, expression cassette, or vector provided herein. In some embodiments, the GA1-m nucleic acid is operably associated with a polynucleotide sequence of interest (e.g., a heterologous polynucleotide sequence of interest). In some embodiments, the polynucleotide sequence of interest is a promoter sequence. In further embodiments, the promoter sequence is a constitutive or inducible promoter sequence. In other embodiments, the polynucleotide sequence of interest is a male- or female-specific promoter sequence described herein or otherwise known in the art. In other embodiments, the GA1-m nucleic acid comprises a GA1-m promoter sequence, or fragment thereof, and the polynucleotide sequence of interest encodes a polypeptide that imparts a desirable agronomic trait to the plant (e.g., drought resistance, heat resistance, salt resistance, disease resistance, insect and other pest resistance [e.g., a Bacillus thuringiensis endotoxin], herbicide resistance, and the like), confers male sterility, improves fertility and/or improves nutritional quality. The disclosure further comprises host plants, cells, plant parts, seeds, tissue culture (including callus) transiently or stably transformed with the GA1-m nucleic acids, expression cassettes or vectors provided herein.

In some embodiments, the method comprises transforming the plant, plant part or plant cell with a GA1-f nucleic acid, expression cassette, or vector provided herein. In some embodiments, the GA1-f nucleic acid is operably associated with a polynucleotide sequence of interest (e.g., a heterologous polynucleotide sequence of interest). In some embodiments, the polynucleotide sequence of interest is a promoter sequence. In further embodiments, the promoter sequence is a constitutive or inducible promoter sequence. In other embodiments, the polynucleotide sequence of interest is a male- or female-specific promoter sequence described herein or otherwise known in the art. In other embodiments, the GA1-f nucleic acid comprises a GA1-f promoter sequence, or fragment thereof, and the polynucleotide sequence of interest encodes a polypeptide that imparts a desirable agronomic trait to the plant (e.g., resistance to an abiotic stress such as drought, heat, and salt; disease resistance, insect and other pest resistance [e.g., a Bacillus thuringiensis endotoxin], herbicide resistance, and the like), confers male sterility, improves fertility and/or improves nutritional quality. The disclosure further comprises host plants, cells, plant parts, seeds, tissue culture (including callus) transiently or stably transformed with the GA1-f nucleic acids, expression cassettes or vectors provided herein.

In some embodiments, the disclosure provides a transgenic plant comprising GA1-f nucleic acid(s) and/or GA1-m nucleic acid(s), and expression cassette(s), and/or vector(s) provided herein. The plant can be transiently or stably transformed with the nucleic acid(s), expression cassette(s) or vector(s). In representative embodiments, the plant comprises a cell or plant part provided herein. In further embodiments, the transgenic plant has a cross-incompatible, cross-compatible, or self-incompatible phenotype.

### Methods:

In some embodiments, the disclosure provides a method of conferring self-incompatibility of a plant, the method comprising:

stably transforming a plant cell with an isolated nucleic(s) acid comprising a polynucleotide sequence selected from the group:
(a1) a polynucleotide sequence encoding a polypeptide comprising the amino acid sequence of

SEQ ID NO:6 (Tcb1-f);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such PME activity;
(d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and/or
(e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
an isolated nucleic(s) acid comprising a polynucleotide sequence selected from the group:
(a2) a polynucleotide sequence encoding a polypeptide comprising the amino acid sequence of SEQ ID NO:6 (Tcb1-f);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
(d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and/or
(e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
regenerating or developing a plant from the transformed plant cell comprising the isolated nucleic acid, and expressing the polynucleotide sequence in the plant, optionally, wherein the plant does not express Tcb1-m. In some embodiments, the plant does not express Tcb1-m or is not teosinte. In some embodiments, the method further comprises using a Tcb1-m plant comprising a polynucleotide sequence encoding a polypeptide comprising the amino acid sequence of SEQ ID NO:2 (Tcb1-m) from a separate plant line (either a natural Tcb1-m line or a transgenic Tcb1-m line) to force cross-pollination and/or hybrid production.

In another embodiment, the disclosure provides a substantially homogeneous population of plants of a predetermined hybrid variety of a crop which is capable of undergoing both self-pollination and cross-pollination comprising: the method comprising:
stably transforming a plant cell with an isolated nucleic(s) acid comprising a polynucleotide sequence selected from the group:
(a1) a polynucleotide sequence encoding a polypeptide comprising the amino acid sequence of SEQ ID NO:6 (Tcb1-f);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such PME activity;
(d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
(e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
an isolated nucleic(s) acid comprising a polynucleotide sequence selected from the group:
(a2) a polynucleotide sequence encoding a polypeptide comprising the amino acid sequence of SEQ ID NO:6 (Tcb1-f);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
(d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
(e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
regenerating or developing a plant from the transformed plant cell comprising the isolated nucleic acid, and expressing the polynucleotide sequence in the plant, optionally, wherein the plant does not express Tcb1-m.

In some embodiments, the methods provided herein further comprise transforming a plant cell with:
(a) a nucleic acid comprising a polynucleotide sequence encoding a polypeptide comprising the amino acid sequence of SEQ ID NO:21 (ZmPME10-1, mat);
(b) a nucleic acid comprising a polynucleotide sequence encoding a polypeptide comprising a fragment of a protein having the amino acid sequence of at least about 150, 200, 250 300, 350, 400, 450, 500, 550, 600, 650, 655, 660, 665, 666, 667, 668, 669, 670, 671, or 672, contiguous amino acids of SEQ ID NO:21, wherein the polypeptide has ZmPME10-1 biological activity such as the ability to bind Tcb1-m (SEQ ID NO:2);
(c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind Tcb1-m (SEQ ID NO:2); and/or
(d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides encoding SEQ ID NO:21.

In some embodiments, the methods provided herein further comprise transforming a plant cell with:
(a1) a polynucleotide sequence encoding a polypeptide comprising the amino acid sequence of

SEQ ID NO:26 (GA2-m mat);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO: 26, wherein the polypeptide has at least one GA2-m biological activity such as PME;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:26, wherein the polypeptide has at least one GA2-m biological activity such as PME activity;
(d1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na) or SEQ ID NO:28 (GA2-m, mat na) or SEQ I DNO: 33; and/or
(a2) a polynucleotide sequence encoding a polypeptide comprising the amino acid sequence of SEQ ID NO:30 (GA2-f mat);
(b2) a polynucleotide sequence encoding a polypeptide comprising a fragment of a protein having the amino acid sequence of at least about 50, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30, wherein the polypeptide has at least one GA2-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f, mat), wherein the polypeptide has at least one one GA2-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 or SEQ ID NO:32 (GA2-f fl, mat na) or SEQ ID NO: 34.

In some embodiments, the methods provided herein further comprise transforming a plant cell with:
(a1) a polynucleotide sequence encoding a polypeptide comprising the amino acid sequence of SEQ ID NO:14 (GA1-m mat);
(b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21;
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
(a2) a polynucleotide sequence encoding a polypeptide comprising the amino acid sequence of SEQ ID NO:18 (GA1-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f; prom, mat, fl, na).

In another embodiment, the disclosure provides a method of overcoming species barriers to enable fertilization and transfer of traits between species, the method comprising stably transforming a plant cell with
an isolated nucleic acid(s) comprising a polynucleotide sequence selected from:
(a) a polynucleotide sequence encoding a polypeptide comprising the amino acid sequence of SEQ ID NO:1 (Tcb1-m) or SEQ ID NO:2 (Tcb1-m);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as (PME activity or the ability to bind ZmPME10-1 having the amino acid sequence of SEQ ID NO:21;
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(e) a polynucleotide sequence encoding a polypeptide comprising the amino acid sequence of SEQ ID NO:6 (Tcb1-f);
(f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
(h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity;
(i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na); and/or
(j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter).

In some embodiments, the method of overcoming species barriers further comprises the step of stably transforming the plant cell with:
(a) a nucleic acid comprising a polynucleotide sequence encoding a polypeptide comprising the amino acid sequence of SEQ ID NO:21 (ZmPME10-1, mat);
(b) a nucleic acid comprising a polynucleotide sequence encoding a polypeptide comprising a fragment of a protein having the amino acid sequence of at least about 150, 200, 250 300, 350, 400, 450, 500, 550, 600, 650, 655, 660, 665, 666, 667, 668, 669, 670, 671, or 672, contiguous amino acids of SEQ ID NO:21, wherein the polypeptide has ZmPME10-1 biological activity such as the ability to bind Tcb1-m (SEQ ID NO:2);
(c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind Tcb1-m (SEQ ID NO:2); and/or
(d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 or SEQ ID NO:4 (ZmPME10-1, fl na).

In some embodiments, the method of overcoming species barriers further comprises the step of stably transforming the plant cell with:
(a1) a polynucleotide sequence encoding a polypeptide comprising the amino acid sequence of

SEQ ID NO:13 or SEQ ID NO:14 (GA1-m mat);
(b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21;
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
(a2) a polynucleotide sequence encoding a polypeptide comprising the amino acid sequence of SEQ ID NO:17 or SEQ ID NO:18 (GA1-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f; prom, mat, fl, na).

In some embodiments, the method of overcoming species barriers further comprises the step of stably transforming the plant cell with:
(a1) a polynucleotide sequence encoding a polypeptide comprising the amino acid sequence of SEQ ID NO:25 or SEQ ID NO:26 (GA2-m mat);
(b1) a polynucleotide sequence encoding a polypeptide comprising a fragment of a protein having the amino acid sequence of at least about 150, 200, 225, 250 275, 300, 325, 350, 360, 311, 362, 363, or 364, contiguous amino acids of SEQ ID NO:26, wherein the polypeptide has at least one GA2-m biological activity such as PME activity or the ability to bind ZmPME10-1 bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:26, wherein the polypeptide has at least one GA2-m biological activity such as PME activity;
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:27 (GA2-m, mat na) or SEQ ID NO:28 (GA2-m, fl na) or SEQ OD NO: 33; and/or
(a2) a polynucleotide sequence encoding a polypeptide comprising the amino acid sequence of SEQ ID NO:29 or SEQ ID NO:30 (GA2-f mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30, wherein the polypeptide has at least one GA2-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:30, wherein the polypeptide has at least one GA2-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:31, or SEQ ID NO:32(GA2-f prom, mat, fl, na) or SEQ ID NO: 34.

In some embodiments, the disclosure provides a method of conferring self-incompatibility of a plant, the method comprising:
stably transforming a plant cell with an isolated nucleic(s) acid comprising a polynucleotide sequence selected from the group:
(a1) a polynucleotide sequence encoding a polypeptide comprising the amino acid sequence of SEQ ID NO:29 (GA2-f, fl) or SEQ ID NO:30 (GA2-f, mat);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30 (GA2-f), wherein the polypeptide has GA2-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such PME activity;
(d1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
(e1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
(f1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
an isolated nucleic(s) acid comprising a polynucleotide sequence selected from the group:
(a2) a polynucleotide sequence encoding a polypeptide comprising the amino acid sequence of SEQ ID NO:29 (GA2-f, fl) or SEQ ID NO:30 (GA2-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30 (GA2-f), wherein the polypeptide has GA2-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such as PME activity;
(d2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
(e2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or (f2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
regenerating or developing a plant from the transformed plant cell comprising the isolated nucleic acid, and expressing the polynucleotide sequence in the plant, optionally, wherein the plant does not express GA2-m. In some embodiments, the plant does not express GA2-m or is not teosinte. In some embodiments, the method further comprises using a GA2-m plant comprising a polynucleotide sequence encoding a polypeptide comprising the amino acid sequence of SEQ ID NO:26 (GA2-m, mat) from a separate plant line (either a natural GA2-m line or a transgenic GA2-m line) to force cross-pollination and/or hybrid production.

In another embodiment, the disclosure provides a substantially homogeneous population of plants of a predetermined hybrid variety of a crop which is capable of undergoing both self-pollination and cross-pollination comprising: the method comprising:
stably transforming a plant cell with an isolated nucleic(s) acid comprising a polynucleotide sequence selected from the group:
(a1) a polynucleotide sequence encoding a polypeptide comprising the amino acid sequence of SEQ ID NO:29 (GA2-f, fl) or SEQ ID NO:30 (GA2-f, mat);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30 (GA2-f), wherein the polypeptide has GA2-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such PME activity;
(d1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
(e1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
(f1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
an isolated nucleic(s) acid comprising a polynucleotide sequence selected from the group:
(a2) a polynucleotide sequence encoding a polypeptide comprising the amino acid sequence of SEQ ID NO:29 (GA2-f, fl) or SEQ ID NO:30 (GA2-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:30 (GA2-f), wherein the polypeptide has GA2-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such as PME activity;
(d2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
(e2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
(f2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
regenerating or developing a plant from the transformed plant cell comprising the isolated nucleic acid, and expressing the polynucleotide sequence in the plant, optionally, wherein the plant does not express GA2-m.

In some embodiments, the methods provided herein further comprise transforming a plant cell with:
(a) a nucleic acid comprising a polynucleotide sequence encoding a polypeptide comprising the amino acid sequence of SEQ ID NO:21 (ZmPME10-1, mat);
(b) a nucleic acid comprising a polynucleotide sequence encoding a polypeptide comprising a fragment of a protein having the amino acid sequence of at least about 150, 200, 250 300, 350, 400, 450, 500, 550, 600, 650, 655, 660, 665, 666, 667, 668, 669, 670, 671, or 672, contiguous amino acids of SEQ ID NO:21, wherein the polypeptide has ZmPME10-1 biological activity such as the ability to bind GA2-m (SEQ ID NO:2);
(c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind GA2-m (SEQ ID NO:2); and/or
(d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 or SEQ ID NO:4 (ZmPME10-1, fl na).

In some embodiments, the methods provided herein further comprise transforming a plant cell with:
(a1) a polynucleotide sequence encoding a polypeptide comprising the amino acid sequence of SEQ ID NO:14 (GA1-m mat);
(b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21;
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
(a2) a polynucleotide sequence encoding a polypeptide comprising the amino acid sequence of SEQ ID NO:18 (GA1-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f; prom, mat, fl, na).

In another embodiment, the disclosure provides a method of overcoming species barriers to enable fertilization and transfer of traits between species, the method comprising stably transforming a plant cell with an isolated nucleic acid(s) comprising a polynucleotide sequence selected from:
(a) a polynucleotide sequence encoding a polypeptide comprising the amino acid sequence of SEQ ID NO:25 (GA2-m, fl) or SEQ ID NO:26 (GA2-m, mat);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m), wherein the polypeptide has at least one GA2-m biological activity such as PME;
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m), wherein the polypeptide has at least one GA2-m biological activity such as PME activity;
(d) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m), wherein the polypeptide has at least one GA2-m biological activity such as PME activity;
(e) a polynucleotide sequence encoding a polypeptide comprising the amino acid sequence of SEQ ID NO:29 (GA2-f, fl) or SEQ ID NO:30 (GA2-f, mat);
(f) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has GA2-f biological activity such as PME activity;
(g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such as PME activity;
(h) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity;
(i) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na);
(j) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:33;
(k) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and/or
(l) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:34; and
regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter).

In some embodiments, the method of overcoming species barriers further comprises the step of stably transforming the plant cell with:
(a) a nucleic acid comprising a polynucleotide sequence encoding a polypeptide comprising the amino acid sequence of SEQ ID NO:21 (ZmPME10-1, mat);
(b) a nucleic acid comprising a polynucleotide sequence encoding a polypeptide comprising a fragment of a protein having the amino acid sequence of at least about 150, 200, 250 300, 350, 400, 450, 500, 550, 600, 650, 655, 660, 665, 666, 667, 668, 669, 670, 671, or 672, contiguous amino acids of SEQ ID NO:21, wherein the polypeptide has ZmPME10-1 biological activity such as the ability to bind GA2-m (SEQ ID NO:2);
(c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind GA2-m (SEQ ID NO:2); and/or
(d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 or SEQ ID NO:4 (ZmPME10-1, fl na).

In some embodiments, the method of overcoming species barriers further comprises the step of stably transforming the plant cell with:
(a1) a polynucleotide sequence encoding a polypeptide comprising the amino acid sequence of

SEQ ID NO:13 or SEQ ID NO:14 (GA1-m mat);
(b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21;
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
(a2) a polynucleotide sequence encoding a polypeptide comprising the amino acid sequence of SEQ ID NO:18 (GA1-f, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f; prom, mat, fl, na).

Having described the present the methods and compositions provided herein, the same will be explained in greater detail in the following examples, which are included herein for illustration purposes only, and which are not intended to be limiting.

Having described the present the methods and compositions provided herein, the same will be explained in greater detail in the following examples, which are included herein for illustration purposes only, and which are not intended to be limiting.

### EXAMPLES

### Example 1. Reproductive Isolation and Mate Rejection in Zea—The Tcb1-Female Gene Encodes a Pectin Methylesterase that in Teosinte Silks Prevents Fertilization by Maize Pollen

Despite being members of the same species, some strains of wild teosinte maintain themselves as a distinct breeding population by blocking fertilization by pollen from neighboring maize plants. These teosinte strains may be in the process of evolving into a separate species, since reproductive barriers that block gene flow are critical components in speciation. This trait is conferred by the Teosinte crossing barrier1-s (Tcb1-s) haplotype, making Tcb1 a speciation gene candidate. Tcb1-s contains a female gene that blocks non-self-type pollen and a male function that enables self-type pollen to overcome that block. The Tcb1-female gene encodes a pectin methylesterase (PME), implying that modification of the pollen cell wall by the pistil is a key mechanism by which these teosinte females reject foreign (but closely related) pollen.

Maize (Zea mays ssp mays) was domesticated from annual teosinte (Zea mays ssp parviglumis) in the Balsas River valley of Mexico (Matsuoka et al., PNAS 99:6080-6084 (2002)). In some locations, sympatric populations of domesticated maize and annual teosinte grow in intimate associate and flower synchronously, but rarely produce hybrids (Kermicle et al., Maydica 35:399-408 (1990)), Evans et al., Theoretical and Applied Genetics 103:259-265 (2001)). In sexually reproducing plants, reproductive barriers exist at different stages, including pre-pollination, post-pollination, and post-fertilization. Post-pollination barriers depend on interaction between the pollen grain and the female reproductive organs (stigma, style, and ovule). In Zea mays, haplotypes at three loci, Gametophyte factor1-s (GA1-male-s), Gametophyte factor2-s (Ga2-s), and Teosinte crossing barrier1-s (Tcb1-s), confer unilateral cross-incompatibility. While GA1-male-s and Ga2-s are widespread in domesticated maize, Tcb1-s is almost exclusively found in wild teosinte populations. The Tcb1-s haplotype, like GA1-male-s and Ga2-s, confers unilateral cross-incompatibility against varieties carrying the tcb1 (or ga1 or ga2) haplotype. Viewed otherwise, Tcb1-s provides a pollen function that overcomes the crossing barrier. The latter view is preferred since pollen containing both Tcb1-s and tcb1 haplotypes fertilizes Tcb1-s plants, indicating that Tcb1-s compatibility is not overcome by the Tcb1-s:tcb1 mismatch, as is also the case for the GA1-male and Ga2 systems (Kermicle et al., Sex Plant Reprod 18:187-194 (2005), Kermicle et al., J Hered 101:737-749 (2010)). Tcb1-s was first described in teosinte subspecies mexicana Collection 48703 from the central and southern Mexico; this strain also contained the male-only haplotype, GA1-male-m, of the GA1-male locus which together with male and female functions of Tcb1, make up the Teosinte Incompatibility Complex (TIC) Kermicle et al., Maydica 35:399-408 (1990), Evans et al., Theoretical and Applied Genetics 103:259-265 (2001)).

Collections of teosinte of both mexicana and parviglumis subspecies from the central Mexican plateau carry Tcb1-s (Kermicle et al., Genetics 172:499-506 (2006)). Tcb1-s confers to females the ability to block fertilization by maize (tcb1 type) pollen by restricting pollen tube growth (Kermicle et al., Plant reproduction 27:19-29 (2014)). In the reciprocal cross, teosinte pollen is able to fertilize maize, although poorly when in competition with maize pollen (Evans et al., Theoretical and Applied Genetics 103:259-265 (2001)). Tcb1 was proposed to be a candidate speciation gene contributing to isolation of diverging maize and teosinte populations, as wild teosinte populations respond to the pressure of cultivated, closely related varieties of domesticated maize (Kermicle et al., Genetics 172:499-506 (2006)).

The male and female functions of Tcb1-s are tightly linked but separable by recombination (Kermicle et al., Plant reproduction 27:19-29 (2014)). Thus, there are four functional classes at this locus (Table 1 for gene content and origin): Tcb1-s has both functional male and female genes, Tcb1-male (Tcb1-m) has only the functional male gene (Kermicle et al., Genetics 172:499-506 (2006), Kermicle et al., Plant reproduction 27:19-29 (2014)).

Tcb1-female (Tcb1-f) has only the functional female gene, and the tcb1 haplotype found in almost all maize lines has neither of the two functional genes. In teosinte, Tcb1-s activity in the silks prevents fertilization by maize (tcb1) pollen, while Tcb1-m activity in pollen enables fertilization of Tcb1-f females (Kermicle et al., Plant reproduction 27:19-29 (2014)).

TABLE 1
Haplotypes of the tcb1 locus

| | | Female | Pollen | Principal |
|---|---|---|---|---|
| Haplotype | Gene Content | Barrier | Penetration | source |
| Tcb1-s | Tcb1-f (Pertunda) | + | + | Teosinte |
| | Tcb1-m | | | |
| Tcb1-f | Tcb1-f (Pertunda) | + | − | Recombination |
| Tcb1-m | Tcb1-m | − | + | Teosinte |
| tcb1 | — | − | − | Maize |

To clone the Tcb1 genes, fine mapping of Tcb1-s:Col48703 haplotype was performed based on a tcb1 backcross population with a population of approximately 15,000 chromosomes. Using maize B73 genome as a reference (Y. Jiao et al., Nature 546:524-527 (2017)), the Tcb1 locus was delimited to a region spanning 480 kb on the short arm of chromosome 4. Within this region, there are eleven annotated genes. However, all of these were ruled out as candidates for Tcb1 functions because they either had identical sequence with identical expression levels between tcb1 and Tcb1-s haplotypes or no expression in the silk or pollen in Tcb1-s or tcb1 (mapping markers included in Table 2). The Tcb1 genes, therefore, are likely absent from the maize genome. This is not surprising considering the widespread structural variations in genomes between maize lines and between teosinte populations (Swanson-Wagner et al., Genome Res 20:1689-1699 (2010)).

TABLE 2
PCR primers for mapping Tcb1-s relative to the maize B73 reference genome

| | | SEQ ID | Marker | |
|---|---|---|---|---|
| Marker | Primer eequences | NO: | types | Note |
| 053600 | FP: AGCAGGTGCCGCCCGT | 8 | Indel | Tcb1-s left border |
| | RP: AAGCGAGGGTTTGCTCGAT | 11 | | |
| 444073up8 | FP: GAGCAGGGTCAGCTGGAAGGAAC | 33 | dCAPS | Tcb1-f left border |
| | RP: GGCATCTCTTGCTTGGCGGGC | 34 | | |
| Gene 0.1up | FP: GATAAGTTTGCAAGTGGCCCATCA | 35 | Indel | Tcb1-m right border |
| | RP: AGAGATGTTCTGCAATGGGCCTA | 36 | | |
| 106164 | FP: GGCTGAAGCAGCAGAGCCATCCTAA | 37 | Indel | Tcb1-s right border |
| | RP: CTGTGTGTGGGATCGGATCCCTA | 38 | | |

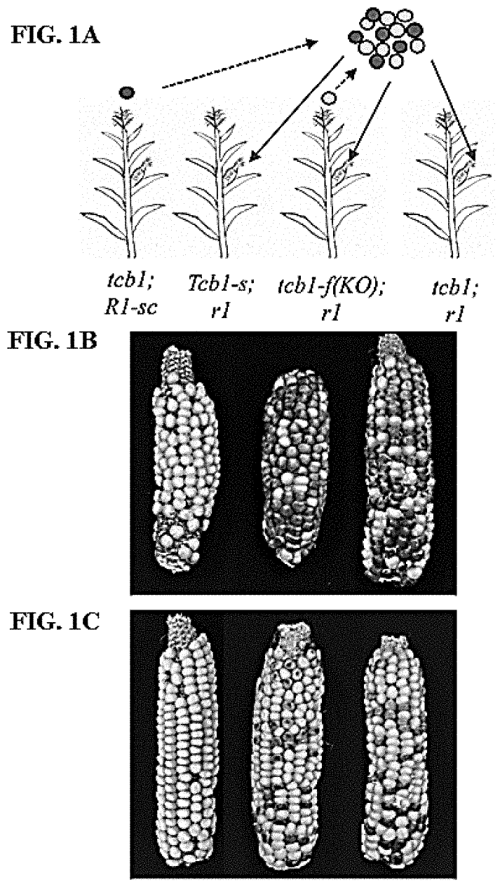

To identify Tcb1-s knockout mutants, maize lines homozygous for the Tcb1-s:Col48703 haplotype and carrying active Mutator transposons were crossed to maize inbred A195 su1. The progeny are expected to be heterozygous for Tcb1-s with su1 approximately 6 cM away and in repulsion (Evans et al., Theoretical and Applied Genetics 103:259-265 (2001)). Due to the rejection of the tcb1 pollen (which is predominantly su1), about 3% of the kernels in every ear with functional Tcb1-s were expected to be sugary in this open-pollinated population, while any ears without a crossing barrier were predicted to segregate su1 at 25%. Out of a population of approximately 6,000 individuals, two exceptional ears were found. One ear segregated for 25.6% sugary. This allele is termed Tcb1-f(KO1). The second isolate contained a sector of about 45 kernels within which the segregation was one-fourth sugary despite sugary segregating at ˜3% over the rest of the ear. This allele is termed Tcb1-f(KO2). Mixed pollination tests with the progeny of both individuals show that the loss of function is heritable, and both variants fertilized a Tcb1-s/tcb1 strain normally, indicating the retention of the male function of Tcb1-s (Tcb1-s mutated, but Tcb1-m intact) (FIG. 1). In the case of Tcb1-f(KO2), progeny of seeds within the loss-of-function side of the ear inherited the knock-out, while those on the other side of the ear inherited fully functional Tcb1-s.

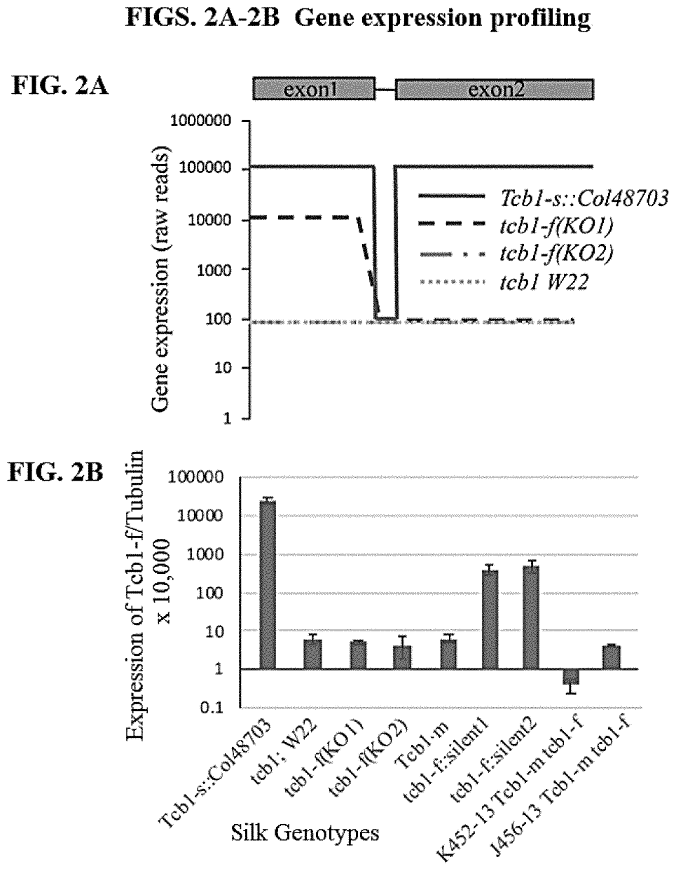

RNA from silks of four genotypes were subjected to short read RNA-seq. Transcript models were assembled de novo from the RNA-seq reads, and expression levels of genes were compared between these two knockout mutants, a standard maize inbred line W22 (genotype tcb1), and a functional Tcb1-s line (a W22 subline to which the Tcb1-s:Co148703 haplotype had been introduced by backcrossing). One gene, named here Tcb1-female encoding a maize pectin methylesterase38 (PME38) homolog (sharing 40% identity), was identified as a candidate for the Tcb-f gene. Tcb1-female is highly expressed in Tcb1-s silks (with a peak read depth of ˜100,000) compared to the standard maize tcb1 W22 silks, Tcb1-f(KO2) silks (maximum read depths of ˜100) and Tcb1-f(KO1) silks (maximum read depth of ˜10,000 for the 5′ end and ˜100 for the 3′ end of the transcript model) (FIG. 2A). Based on the mRNA sequence, PCR primers were designed to isolate a BAC (Bacteria Artificial Chromosome) clone from a library we constructed from a maize line to which the Tcb1-s:Col48703 haplotype had been introduced by backcrossing. By comparing mRNA and gene sequences, a 99-base intron was identified in Tcb1-female (Tcb1-f), which explains the gap between the two signal peaks in Tcb1-s. The intron region showed the same level of expression as that from the whole gene region in Tcb1-f(KO2) and in the W22 maize line and in the downstream gene region in Tcb1-f(KO1). qRT-PCR confirmed this expression difference (FIG. 2B). Tcb1-female is not present in the maize B73 reference genome, which is consistent with the mapping data, and the closest homologs of Tcb1-female are located at the gal locus (Moran et al., Front. Plant Sci. 8:1926 (2017)).

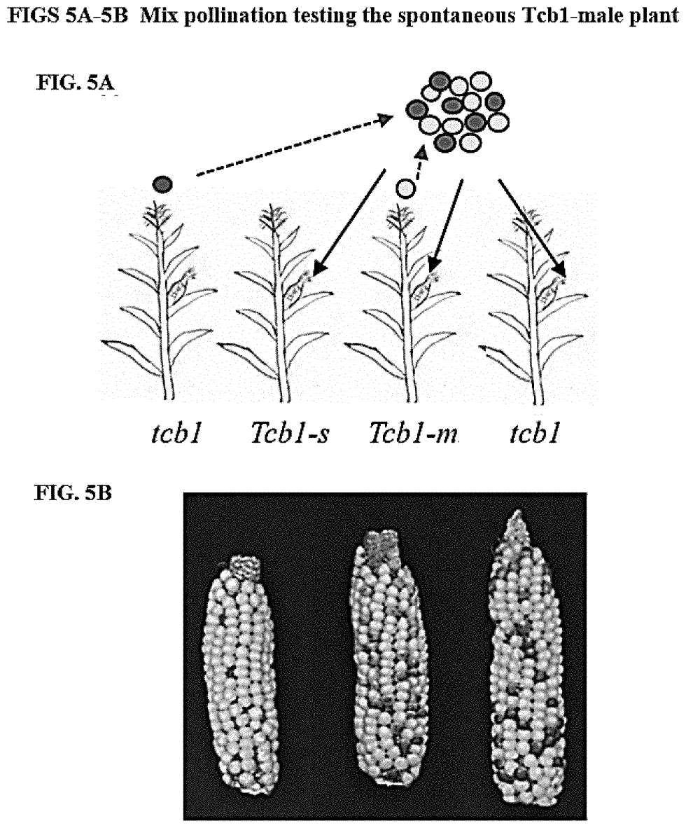

In addition to the two knockout mutants from the active Mutator transposon population, several additional lines derived from the Tcb1-s:Col48703 accession have lost female barrier function. One was recovered during early backcrossing of the Tcb1-s:Col48703 haplotype into maize (Kermicle et al., Maydica 35:399-408 (1990)). Mixed pollination confirmed this is a Tcb1-m only plant (FIGS. 5A-5B). Additionally, two independent Tcb1-s lines were isolated in which the barrier gradually lost the strength during ten generations of backcrossing to maize and selection for Tcb1-m function (Kermicle et al., Plant reproduction 27:19-29 (2014)). These two lines were named as Tcb1-f:silent lineage1 (Tcb1-f:sl1) and Tcb1-f:silent lineage2 (Tcb1-f:sl2), based on the “progressive” manner of the barrier weakening. Tcb1-female has much lower expression in these three additional tcb1 lines, as shown with qRT-PCR (FIG. 2B). Expression of Tcb1-female was also tested on the two Tcb1-m recombinants from the mapping population, which have lost the Tcb1-female gene by recombination. Again, Tcb1-female expression was much lower than in Tcb1-s lines (FIG. 2B).

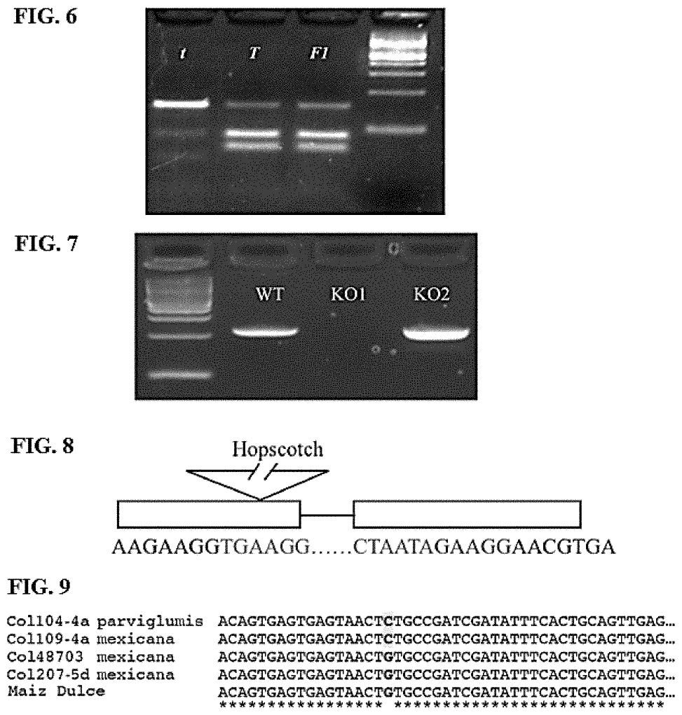

Using a PCR-based dCAPS (Derived Cleaved Amplified Polymorphic Sequence) marker designed for the Tcb1-female gene, it was shown that Tcb1-female maps to the tcb1 locus (FIG. 6). This marker was then tested on the fifteen closest recombinants from the mapping population of ˜15,000 individuals (including four recombinants between the Tcb1-s and Tcb1-m genes) (Kermicle et al., Plant reproduction 27:19-29 (2014)). Of the fifteen plants, six carried Tcb1-s and blocked maize pollen, and nine lacked the barrier. Results showed that all the six recombinants that carry the barrier had the Tcb1-female gene, while in all nine recombinants that are receptive to maize pollen, Tcb1-female was absent. This shows a perfect physical linkage between Tcb1-female and the Tcb1-s barrier function. RNA-seq data suggest that the mutation in Tcb1-female occurred somewhere in the first exon in the Tcb1-f(KO1) mutant (FIG. 2A). PCR data confirmed there was a disruption within the coding region of Tcb1-female in KO1 (FIG. 7). Quite differently, in Tcb1-f(KO2) mutant silk RNA-seq reads had the same low level of expression as tcb1 silks along the whole Tcb1-female transcript. Whole genome resequencing of both mutants identified a Hopscotch retrotransposon insertion in the first exon in Tcb1-f(KO1), close to the site where Tcb1-female expression drops sharply. PCR spanning both ends of the insertion confirmed the insertion event and the border sequences (FIG. 8). In contrast, in Tcb1-f(KO2), Tcb1-female was fully assembled, consistent with the PCR data that the coding region is present (FIG. 7). The tcb1-f(KO2) allele then could either be mutated in a regulatory region, potentially hundred kilobases away from the coding region, or could be an epi-allele. Similarly, no mutations were found in the coding region of Tcb1-female in the Tcb1-m line or the Tcb1-f:sl1 or Tcb1-f:sl2 lines described above.

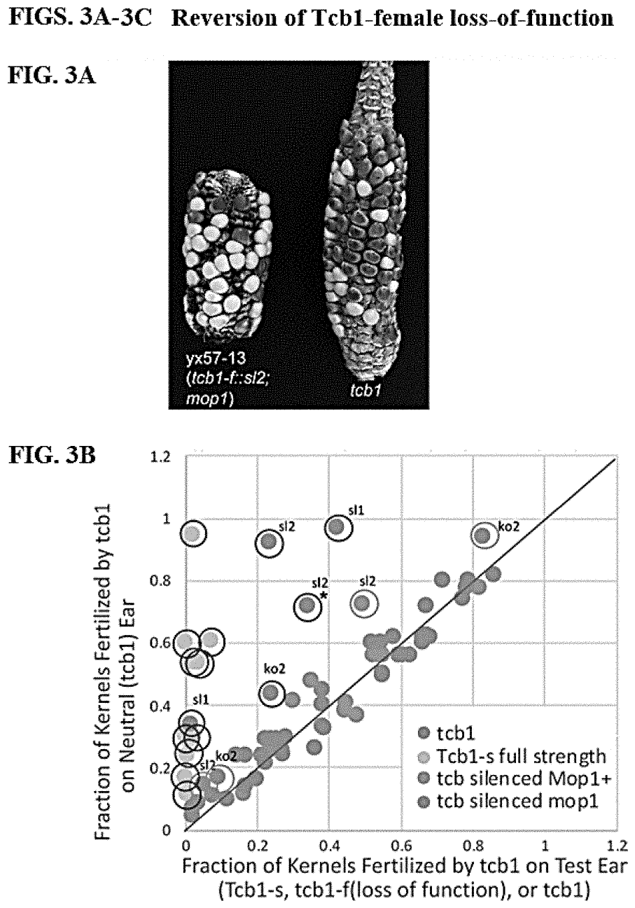

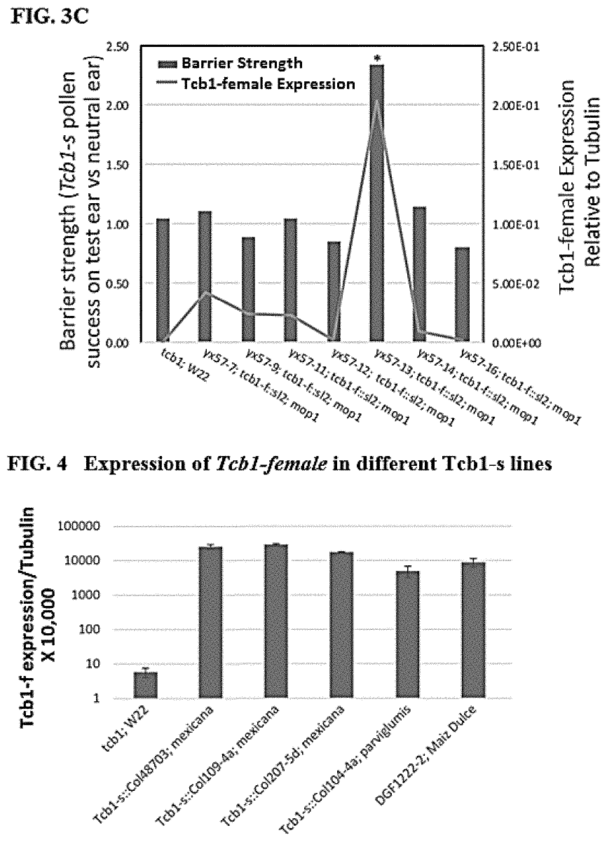

The Tcb1-f(KO2), Tcb1-f:sl1, and Tcb1-f:sl2 lines were tested for reversion to Tcb1-s in double mutants with mediator of paramutation1 (mop1) mutation. MOP1 encodes a RNA-dependent RNA polymerase and is a key component of RNA-directed DNA Methylation (Alleman et al., Nature 442:295-298 (2006)). mop1 mutations reactivate silenced genes and affect broad developmental programs (Dorweiler et al., Plant Cell 12:2101-2118 (2000)). Re-activation of the Tcb1-s function was rare; in only ˜14-22% of the mop1 females tested, did the loss-of-function plants show some recovery of Tcb1-s function. Pollen competition experiments were performed for full strength Tcb1-s females, tcb1 females, and the Tcb1-female loss of function lines without sequence changes (primarily Tcb1-f(KO2), Tcb1-f:sl1, and Tcb1-f:sl2) (FIG. 3A). All of the Tcb1-s ears tested showed strong preference for Tcb1-s pollen (0-7% kernels from tcb1 pollen regardless of the ratio of the two pollen types in the mix as indicated by the neutral ear) with the kernel ratio on the test ear and control ear being different from each other at p<0.0001 (Fisher exact test) (FIG. 3B). Of the 36 mop1; Tcb1-female loss of function females tested only one had as strong of a pollen preference as full strength Tcb1-s females, but five had a difference between the test and control ears at p<0.0001 and an additional three females could be included if the stringency was relaxed to p<0.01 (FIG. 3B). These partial revertants included plants of lines Tcb1-f(KO2), Tcb1-f:sl1, and Tcb1-f:sl2. Of the twelve loss of function plants tested that were heterozygous wild-type for mop1, none of the plants passed the more stringent p<0.0001 threshold and one passed the less stringent p<0.01 threshold. It may be that maintaining the silenced derivatives of Tcb1-female with mop1 for multiple generations would increase the revertant frequency.

A subset of homozygous mopl Tcb1-f:sl2 plants were tested at random for Tcb1-female expression in silks prior to pollination. Among the seven tested plants, one plant, yx57-13, showed about four hundred fold higher expression compared to that of the standard W22 maize and eight times higher than Tcb1-f:sl2 plants (FIG. 3C). This plant was the only one of those tested for Tcb1-female expression that recovered the ability to reject tcb1 pollen, although not as efficiently as full strength Tcb1-s plants, which have still higher expression of Tcb1-female than this revertant. This indicates a correlation between Tcb1-female expression level and the female barrier strength, and further supports Tcb1-female as the Tcb1-s gene.

In addition to the Tcb1-s:Col48703 strain descried above, three other teosinte-derived Tcb1-s lines, two from ssp. mexicana and one ssp. parviglumis (Kermicle et al., Genetics 172:499-506 (2006)), were tested for Tcb1-female expression in silk tissue. In all three lines, Tcb1-female expression levels are extremely high and comparable to that of the original central plateau TIC haplotype Tcb1-s:Col48703 (FIG. 4). Interestingly, even though none of the modern north American maize lines tested to date carry the Tcb1-s haplotype, an ancient Maiz Dulce variety, Jalisco78 line 1222-2, grown at intermediate altitudes in southwestern Mexico carries Tcb1-s (Jones et al., Euphytica 209:63-69 (2016)). This is a specialty line that may have undergone selection for cross-incompatibility factors similarly to GA1-male-s in maize popcorn lines. Whether this Maiz Dulce line acquired Tcb1-s from nearby teosinte populations during its origin is unknown, but maize lines from this region have been shown to have substantial introgression from mexicana teosintes (van Heerwaarden et al., PNAS 108:1088-1092 (2011)). Predicted Tcb1-female coding sequences are identical in all five Tcb1-s lines: three mexicana accessions, one parviglumis accession, and the Maiz Dulce line. One Single Nucleotide Polymorphism (SNP) in the intron separates these lines into two groups: one group including the parviglumis line (Col104-4a) and one mexicana line (Col109-4a), and the other group including two mexicana lines (Col48703 and Col207-5d) and the Maiz Dulce line (FIG. 9).

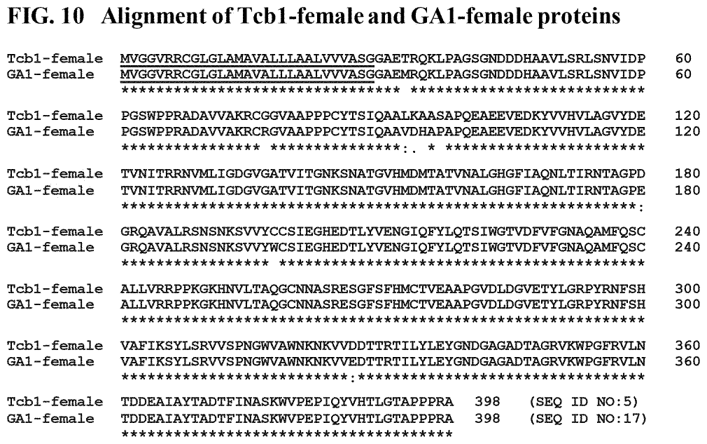

The most similar gene to Tcb1-female is a candidate PME gene for GA1-male-female function. This gene, termed TIC (GA1-f), was found to be expressed in the silks of GA1-male-s, but not in ga1 silks, and GA1-f was located to the GA1-male mapping region (Moran et al., Front. Plant Sci. 8:1926 (2017)). Alignment of the TIC and Tcb1-female showed that the two PMEs differ in nine amino acids (FIG. 10). The number of polymorphisms (15 of 1296 nucleotides) between Tcb1-female and TIC suggests that these two genes diverged approximately 175,000 years ago, well before the split between the mexicana and parviglumis subspecies of teosinte and just before the split between Zea mays parviglumis and Zea luxurians, using calculated nucleotide substitution rates for maize (Clark et al., Mol. Biol. Evol. 22:2304-2312 (2005)) and a calculated time since the split between mexicana and parviglumis of ˜60,000 years and parviglumis and luxurians of ˜140,000 years (Ross-Ibarra et al., Genetics 181:1399-1413 (2009)). It will be interesting to test whether the Tcb1-m and GA1-male-male genes diverged at a similar time, suggesting they were already adjacent before divergence.

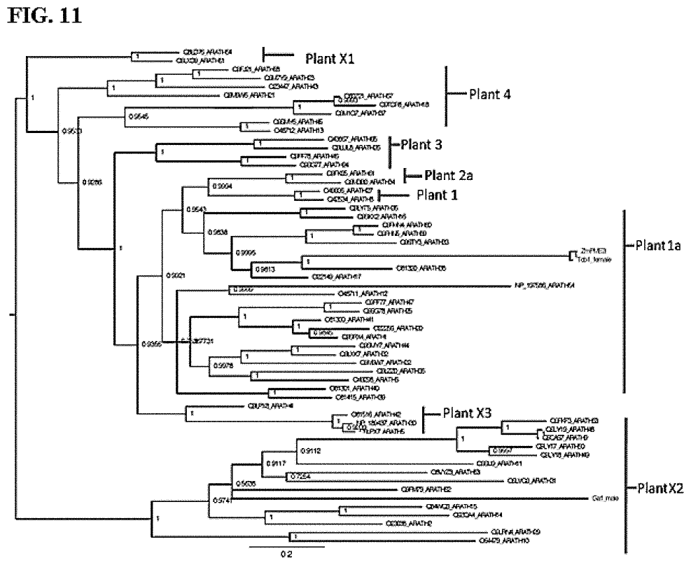

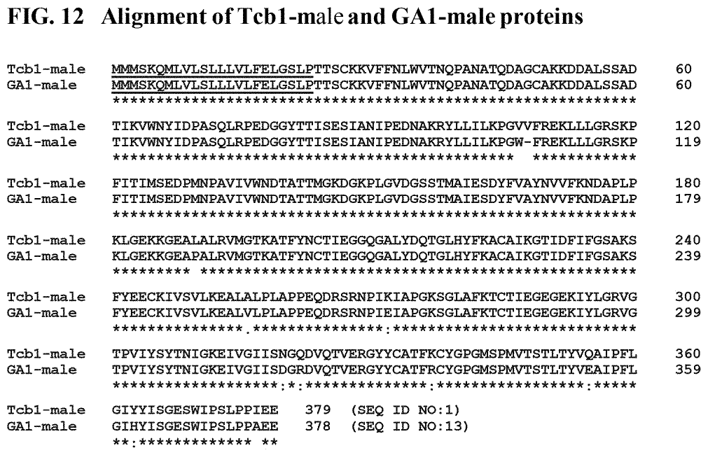

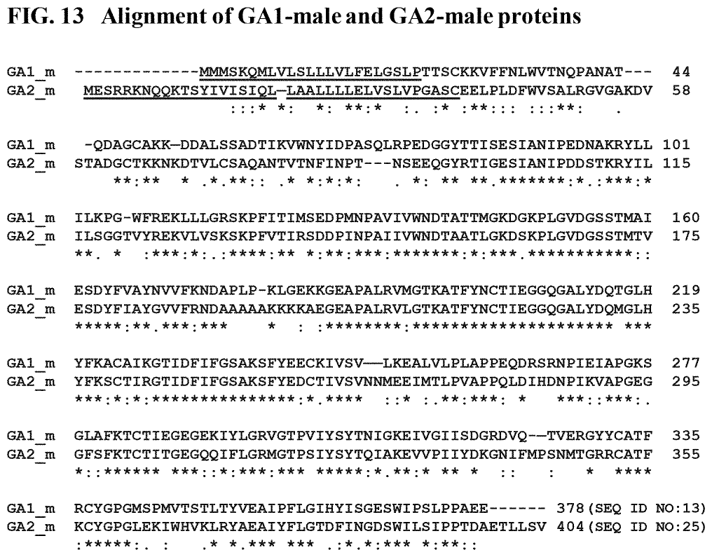

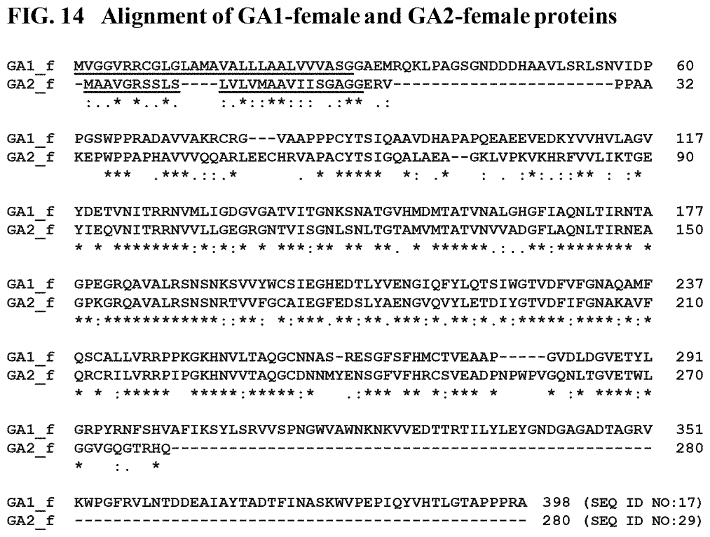

The Tcb1 and GA1-male barriers may share a similar mechanism, but because they are mostly cross-incompatible with one another they likely differ in their interacting partners. However, Tcb1-s and GA1-male-s are not fully cross-incompatible. In situations where pollen rejection is not absolute, Tcb1-s pollen has a competitive advantage over tcb1 pollen on GA1-male-s or Ga2-s silks. This is true for all combinations of interactions between crossing barrier loci (Evans et al., Theoretical and Applied Genetics 103:259-265 (2001), Kermicle et al., J Hered 101:737-749 (2010)) and is consistent with them encoding related proteins, although the behavior of pollen tubes during rejection by each system is slightly different (Kermicle et al., Plant Reproduction 27:19-29 (2014)). Tcb1-female encodes a group 1 type of PME without an N-terminal Pectin Methylesterase Inhibitor (PMEI) domain (Pelloux et al., Trends Plant Sci 12:267-277 (2007)), and contains a predicted signal peptide, so it has the potential to be secreted and interact directly with the pollen tube to remove methyl-esters from the pectin wall of the pollen tube. Esterified pectins are typically associated with the tip of the growing pollen tube, while de-esterified pectins are enriched distally, and there is a correlation between pectin de-esterification and increased cell wall stiffness (Parre et al., Planta 220:582-592 (2005)). Pollen tubes have a “soft tip-hard shell” structure, in that the tip region of the tube cell wall has a single pectin layer that is strong enough to withstand turgor pressure, but plastic enough to allow cell expansion and growth (Steer et al., New Phytologist 111:323-358 (1989)). Inside pollen tubes, pectin is synthesized and esterified in Golgi compartments before delivery to the tip cell wall via vesicle trafficking (Cheung et al., Annu Rev Plant Biol 59:547-572 (2008)), where it can be de-esterified by PMEs (Micheli et al., Trends Plant Sci 6:414-419 (2001)). Pollen cells finely tune the stiffness of the tip cell wall to sustain pollen tube elongation. Either under- or over-supply of PME activity can result in disturbed pollen tube growth and compromised male fertility (Bosch et al., Plant Physiol 138:1334-1346 (2005), Tian et al., Dev Biol 294:83-91 (2006), Rockel et al., Plant J 53:133-143 (2008), Sanati Nezhad et al., Plant J 80, 185-195 (2014)). The TCB1-FEMALE (and ZMPME3) protein falls into the Plant 1a clade of mature PME enzymes (Markovic et al., Carbohydr. Res. 339:2281-2295 (2004)) (FIG. 11).

In summary, genetic and genomic data identify Tcb1-female as the Tcb1-female barrier gene. Teosinte lines carrying Tcb1-female block maize pollen that lacks the male function provided by Tcb1-m. That the Tcb1-s gene encodes a cell wall modifying enzyme is consistent with the model that incompatibility with tcb1 occurs via incongruity rather than active targeting of a Tcb1-m encoded protein (Kermicle et al., Sex. Plant Reprod 18:187-194 (2005)). It will be interesting to test how universal this barrier mechanism is among sexual reproducing plants. Surprisingly, it was shown that another PME family member is encoded by the GA1-male-male gene (Zhang et al., Nat Commun 9:3678 (2018)) (in a very distinct clade, Plant X2, of PME enzymes), raising the possibility that the biochemical barrier to pollen and the ability of pollen to overcome that barrier are conferred by different classes of PME proteins.

The grass family is known to have widely distributed self-incompatibility (SI) among species, however, the molecular nature of the SI genes and how it is related to interspecific cross-incompatibility are not known (Heslop-Harrison et al., Science 215:1358-1364 (1982), Yang et al., New Phytol. 178:740-753 (2008)). The grasses also have an unusually high species diversity for a family with abiotic pollinators (Dodd et al., Evolution 53:732-744 (1999)). Identification of the Tcb1-female gene may facilitate research into the mechanisms of speciation in the grasses. Agriculturally, this work may help managing specialty crop populations by preventing pollen contamination. It may also facilitate development of breeding tools to enrich crop genetic pools by backcrossing crops to their ancestors for the purposes of yield increase or enhanced stress resistance.

### Materials and Methods

Maize and Teosinte Lines and Growth Conditions

All maize and teosinte lines used in this study have been described previously (Kermicle et al., Genetics 172:499-506 (2006), Kermicle et al., Plant reproduction 27:19-29 (2014), Jones et al., Euphytica 209:63-69 (2016)). Plants were grown under field conditions at either Stanford, California or Madison, Wis.

Tcb1-s Mapping

As described before (Evans et al., Theoretical and Applied Genetics 103:259-265 (2001)), a Central Plateau teosinte collection 48703 (Wilkes, Teosinte: the closest relative of maize. Bussey Institution of Harvard University, Cambridge, Massachusetts, (1967)) carrying the Tcb1-s barrier was backcrossed to the Mid-western US dent inbred W22 to incorporate the Tcb1-s locus into a maize background. This Tcb1-s strain was crossed to a chromosome 4 maize tester line virescent17 (v17) brown midrib3 (bm3) sugary1 (su1), and the F1 was then backcrossed to the same tester line. Recombinants carrying crossovers between the four visual markers were tested for the Tcb1-s male and female functions in reciprocal crosses with Tcb1-s/su1 F1 plants. PCR mapping markers were developed to refine the location of crossovers in these recombinants

Tcb1-s Knockout Mutant Screen

To identify loss-of-function mutants of Tcb1-female, a GA1-m Tcb1-s active Mutator strain was crossed to maize inbred A195 su1 (tcb1), and then the progeny were grown as an open-pollinated block. Most of the progeny are expected to be heterozygous for Tcb1-s and su1 in repulsion with su1 approximately 6 cM away from the tcb1 locus (Evans et al., Theoretical and Applied Genetics 103:259-265 (2001)). Due to the rejection of the tcb1 pollen (which is predominantly su1), about 3% of the kernels in every ear with functional Tcb1-s are expected to be sugary in this open-pollinated population, while those without a crossing barrier were predicted to segregate su1 at 25%.

Mixed Pollination Experiments

For the mixed pollination testing of the two Tcb1-female knockout mutants, two pollen donor lines and three pollen receiver lines were used. Pollen from a maize line (tcb1) that does not have the Tcb1 barrier genes but carries the endosperm color marker R1-self color (R1-sc) will produce purple kernel after fertilization of the lines used, while pollen from the knockout plants and the Tcb1-m plants carry r1-r and produce anthocyaninless kernels that are white or yellow. After being collected from the two donors and mixed, pollen was put on the three receiving ears: (1) a Tcb1-s tester ear was used to verify the presence of the Tcb1 male function from the Tcb1-f:KO pollen; (2) the Tcb1-female (KO) ear was used to test the presence/absence of the female barrier function in the knockout mutant; and (3) a maize (tcb1) neutral ear was used to assay the percentage of viable pollen grains from the two donors in the mixture. The same protocol was used on the spontaneous Tcb1-m plant, except the Tcb1-m plant being tested was substituted for the KO plant. For mixed pollinations of the Tcb1-f:silent lineage mopl double mutant plants, pollen from the same R1-sc tcb1 line and a r1-r Tcb1-s tester line was collected, mixed, and applied to the individual silent line ears and the neutral maize ears.

Silk Tissue Collection, RNA Isolation and cDNA Synthesis

Plants for RNA isolation were grown in summer field conditions in Stanford, Calif. Silk tissues were collected around 11 am, immediately put into liquid nitrogen in the field, and stored at −80° C. Total RNA was isolated from silks with Trizol reagent (Invitrogen), DNase-treated, and either subjected to Illumina short read paired end RNA-seq, or used to synthesize the 1st strand cDNA with the Superscript IV RT kit (Invitrogen).

Quantitative RT-PCR

Each line/genotype had three biological replicates, and each in turn had three technical repeats. Tubulin (Zm00001d033850) was used as a reference gene. In each line, relative expression level of Tcb1-female was obtained by comparing Tcb1-female to tubulin.

Sequencing, Assembly and Analysis

All the RNA and DNA sequencing works were done with Illumina Paired-end sequencing by Novogene (CA, USA). RNA-seq reads from all samples were combined and de novo assembled with Trinity v2.4.0 (Grabherr et al., Nat Biotechnol 29:644-652 (2011)) The gene in contig DN33598_c7_g3_i1 was identified as the Tcb1-s candidate gene due to its extremely high expression in the functional Tcb1-s line and the almost no expression in the KO mutants and a standard W22 maize line. PCR primers were designed based on the DN33598_c7_g3_i1 sequence, and one BAC clone was fished out from library made from maize line into which the Tcb1-s:Col48703 haplotype had been introgressed. The BAC sequencing reads were assembled with SPAdes v3.11.1 (Bankevich et al., J. Comput. Biol. 19:455-477 (2012)). NODE_62, a contig that is 13656 bp with coverage of 4029, was identified as having the Tcb1-s candidate gene. Whole genome sequencing reads from the two KO mutants were individually assembled with SPAdes v3.11.1 and BLASTed against NODE_62. Also, the mutant sequencing reads were mapped against NODE_62 using GSNAP (Wu et al., Bioinformatics 26:873-881 (2010)). Combining both approaches identified the hopscotch retrotransposon insertion in the Tcb1-f(KO1) mutant allele.

For phylogenetic analyses, alignments were made using the ClustalW algorithm in MegAlign (DNASTAR™). The predicted mature PME enzymes and the Arabidopsis PME family members were taken from Markovic and Janecek, as were the subfamily designations (Markovic et al., Carbohydr. Res. 339:2281-2295 (2004)). Phylogenies were produced from these alignments using MrBayes v3.2.0 using default settings for amino acid analysis (Huelsenbeck et al., Bioinformatics 17:754-755 (2001)). The MrBayes analysis was performed for 4,100,000 generations at which point the standard deviation of the split frequencies was below 0.004.

Exemplary Sequences

| Description | Sequence |
|---|---|
| Tcb1-male | MMMSKQMLVLSLLLVLFELGSLPTTSCKKVFFNLWVINQPANATQDAGCAKKDDAL |
| Full-Length | SSADTIKVWNYIDPASQLRPEDGGYTTISESIANIPEDNAKRYLLILKPGVVFREK |
| (aa) | LLLGRSKPFITIMSEDPMNPAVIVWNDTATTMGKDGKPLGVDGSSTMAIESDYFVA |
| | YNVVEKNDAPLPKLGEKKGEALALRVMGTKATFYNCTIEGGQGALYDQTGLHYFKA |
| | CAIKGTIDFIFGSAKSFYEECKIVSVLKEALALPLAPPEQDRSRNPIKIAPGKSGL |
| | AFKTCTIEGEGEKTYLGRVGTPVIYSYTNIGKEIVGIISNGQDVQTVERGYYCATF |
| | KCYGPGMSPMVTSTLTYVQAIPFLGIYYISGESWIPSLPPIEE (SEQ ID |
| | NO: 1) |
| Tcb1-male | TTSCKKVFFNLWVTNQPANATQDAGCAKKDDALSSADTIKVWNYIDPASQLRPEDG |
| Mature (aa) | GYTTISESIANIPEDNAKRYLLILKPGVVFREKLLLGRSKPFITIMSEDPMNPAVI |
| | VWNDTATTMGKDGKPLGVDGSSTMAIESDYFVAYNVVFKNDAPLPKLGEKKGEALA |
| | LRVMGTKATFYNCTIEGGQGALYDQTGLHYFKACAIKGTIDFIEGSAKSFYEECKI |
| | VSVLKEALALPLAPPEQDRSRNPIKIAPGKSGLAFKTCTIEGEGEKTYLGRVGTPV |
| | IYSYTNIGKEIVGIISNGQDVQTVERGYYCATFKCYGPGMSPMVTSTLTYVQAIPF |
| | LGIYYISGESWIPSLPPIEE (SEQ ID NO: 2) |
| Tcb1-male | ATGATGATGAGTAAACAAATGCTCGTCTTGTCCCTGCTCCTAGTGTTGTTCGAGCT |
| Full-Length | TGGATCGCTGCCGACGACATCGTGCAAAAAGGTCTTTTTCAACTTATGGGTGACAA |
| (na) | ACCAGCCAGCTAATGCCACCCAAGATGCGGGGTGTGCTAAGAAAGATGATGCGCTC |
| | TCCTCCGCCGACACCATTAAGGTATGGAATTACATCGACCCTGCCTCTCAATTGAG |
| | ACCTGAAGATGGCGGTTACACGACCATTAGCGAGTCCATCGCCAACATCCCTGAGG |
| | ACAACGCCAAACGCTACCTCCTTATCCTCAAACCTGGTGTTGTGTTCCGCGAGAAG |
| | CTGTTACTCGGTAGAAGCAAGCCTTTCATCACCATAATGTCCGAGGACCCCATGAA |
| | CCCTGCTGTTATCGTCTGGAATGACACTGCCACCACCATGGGCAAGGACGGCAAGC |
| | CCCTTGGTGTGGATGGAAGCAGCACCATGGCCATAGAGTCCGACTATTTTGTCGCC |
| | TACAACGTTGTCTTCAAGAACGATGCACCGCTACCAAAGCTAGGGGAAAAGAAAGG |
| | TGAGGCACTAGCACTGCGAGTGATGGGAACAAAGGCAACCTTCTACAATTGCACCA |
| | TCGAAGGCGGCCAGGGTGCTCTGTACGACCAGACGGGTCTGCACTACTTCAAGGCT |
| | TGTGCCATCAAGGGAACCATCGACTTCATCTTCGGATCTGCCAAGTCATTTTATGA |
| | GGAATGCAAAATCGTTTCGGTGTTGAAGGAGGCATTGGCATTGCCATTGGCACCAC |
| | CGGAGCAGGACCGCTCTAGAAATCCCATCAAAATCGCCCCAGGAAAGAGCGGGTTG |
| | GCATTCAAGACTTGCACAATCGAGGGGGAAGGAGAAAAAATTTACTTGGGTAGGGT |
| | GGGCACGCCTGTGATCTACTCCTACACCAATATAGGTAAGGAGATTGTAGGCATAA |
| | TATCTAATGGTCAGGACGTCCAGACTGTCGAAAGGGGGTACTACTGCGCCACTTTC |
| | AAGTGTTACGGGCCTGGGATGTCTCCAATGGTAACCTCAACTCTGACCTATGTCCA |
| | GGCAATACCCTTTCTCGGGATATACTACATCTCGGGGGAGTCGTGGATCCCGTCCC |
| | TACCACCCATTGAAGAATAA (SEQ ID NO: 3) |
| Tcb1-male | ACGACATCGTGCAAAAAGGTCTTTTTCAACTTATGGGTGACAAACCAGCCAGCTAA |
| Mature (na) | TGCCACCCAAGATGCGGGGTGTGCTAAGAAAGATGATGCGCTCTCCTCCGCCGACA |
| | CCATTAAGGTATGGAATTACATCGACCCTGCCTCTCAATTGAGACCTGAAGATGGC |
| | GGTTACACGACCATTAGCGAGTCCATCGCCAACATCCCTGAGGACAACGCCAAACG |
| | CTACCTCCTTATCCTCAAACCTGGTGTTGTGTTCCGCGAGAAGCTGTTACTCGGTA |
| | GAAGCAAGCCTTTCATCACCATAATGTCCGAGGACCCCATGAACCCTGCTGTTATC |
| | GTCTGGAATGACACTGCCACCACCATGGGCAAGGACGGCAAGCCCCTTGGTGTGGA |
| | TGGAAGCAGCACCATGGCCATAGAGTCCGACTATTTTGTCGCCTACAACGTTGTCT |
| | TCAAGAACGATGCACCGCTACCAAAGCTAGGGGAAAAGAAAGGTGAGGCACTAGCA |
| | CTGCGAGTGATGGGAACAAAGGCAACCTTCTACAATTGCACCATCGAAGGCGGCCA |
| | GGGTGCTCTGTACGACCAGACGGGTCTGCACTACTTCAAGGCTTGTGCCATCAAGG |
| | GAACCATCGACTTCATCTTCGGATCTGCCAAGTCATTTTATGAGGAATGCAAAATC |
| | GTTTCGGTGTTGAAGGAGGCATTGGCATTGCCATTGGCACCACCGGAGCAGGACCG |
| | CTCTAGAAATCCCATCAAAATCGCCCCAGGAAAGAGCGGGTTGGCATTCAAGACTT |
| | GCACAATCGAGGGGGAAGGAGAAAAAATTTACTTGGGTAGGGTGGGCACGCCTGTG |
| | ATCTACTCCTACACCAATATAGGTAAGGAGATTGTAGGCATAATATCTAATGGTCA |
| | GGACGTCCAGACTGTCGAAAGGGGGTACTACTGCGCCACTTTCAAGTGTTACGGGC |
| | CTGGGATGTCTCCAATGGTAACCTCAACTCTGACCTATGTCCAGGCAATACCCTTT |
| | CTCGGGATATACTACATCTCGGGGGAGTCGTGGATCCCGTCCCTACCACCCATTGA |
| | AGAATAA (SEQ ID NO: 4) |
| Tcb1-female | MVGGVRRCGLGLAMAVALLLAALVVVASGGAETRQKLPAGSGNDDDHAAVLSRLSN |
| Full-Length | VIDPPGSWPPRADAVVAKRCGGVAAPPPCYTSIQAALKAASAPQEAEEVEDKYVVH |
| (aa) | VLAGVYDETVNITRRNVMLIGDGVGATVITGNKSNATGVHMDMTATVNALGHGFIA |
| | QNLTIRNTAGPDGRQAVALRSNSNKSVVYCCSIEGHEDTLYVENGIQFYLQTSIWG |
| | TVDFVFGNAQAMFQSCALLVRRPPKGKHNVLTAQGCNNASRESGFSFHMCTVEAAP |
| | GVDLDGVETYLGRPYRNFSHVAFIKSYLSRVVSPNGWVAWNKNKVVDDTTRTILYL |
| | EYGNDGAGADTAGRVKWPGFRVLNTDDEATAYTADTFINASKWVPEPIQYVHTLGT |
| | APPPRA (SEQ ID NO: 5) |
| Tcb1-female | GAETRQKLPAGSGNDDDHAAVLSRLSNVIDPPGSWPPRADAVVAKRCGGVAAPPPC |
| Mature (aa) | YTSIQAALKAASAPQEAEEVEDKYVVHVLAGVYDETVNITRRNVMLIGDGVGATVI |
| | TGNKSNATGVHMDMTATVNALGHGFIAQNLTIRNTAGPDGRQAVALRSNSNKSVVY |
| | CCSIEGHEDTLYVENGIQFYLQTSIWGTVDFVFGNAQAMFQSCALLVRRPPKGKHN |
| | VLTAQGCNNASRESGFSFHMCTVEAAPGVDLDGVETYLGRPYRNFSHVAFIKSYLS |
| | RVVSPNGWVAWNKNKVVDDTTRTILYLEYGNDGAGADTAGRVKWPGFRVLNTDDEA |
| | IAYTADTFINASKWVPEPIQYVHTLGTAPPPRA (SEQ ID NO: 6) |
| Tcb1- | ATGGTAGGCGGCGTGAGGAGGTGCGGCCTGGGCCTGGCCATGGCGGTGGCCCTGCT |
| female | GCTCGCTGCGCTGGTTGTCGTTGCAAGCGGCGGCGCGGAGACGAGGCAGAAGCTGC |
| Full- | CTGCTGGCAGTGGTAACGACGACGACCACGCAGCCGTTCTCAGCCGCCTGTCCAAC |
| Length | GTCATTGATCCGCCGGGGAGCTGGCCTCCGCGTGCGGATGCTGTCGTGGCGAAGCG |
| (na) | GTGCCGCGGCGTCGCTGCTCCGCCGCCTTGCTACACCAGCATCCAGGCAGCCGTGG |
| | ATCACGCACCAGCACCACAAGAAGCCGAGGAGGTGGAGGACAAGTACGTCGTGCAT |
| | GTGCTCGCTGGCGTCTACGACGAGACCGTCAACATAACAAGAAGGAACGTGATGCT |
| | GATCGGCGATGGGGTCGGTGCCACCGTCATCACGGGGAACAAGAGTAATGCAACAG |
| | GCGTCCACATGGACATGACGGCGACAGTGAATGCCTTGGGTCACGGCTTCATAGCA |
| | CAGAACCTGACAATCAGAAACACGGCAGGGCCGGAAGGCAGGCAGGCCGTGGCGCT |
| | AAGGTCAAATTCGAACAAGTCGGTCGTCTACTGGTGCAGCATTGAAGGTCATGAGG |
| | ACACCTTGTACGTGGAGAACGGGATCCAGTTCTACCTGCAGACCTCGATCTGGGGC |
| | ACCGTGGACTTTGTGTTTGGCAATGCCCAGGCCATGTTCCAGAGCTGCGCGCTGCT |
| | GGTGCGCCGCCCACCGAAAGGCAAGCACAATGTGCTGACGGCCCAGGGCTGCAACA |
| | ACGCAAGCCGCGAGTCCGGCTTCTCGTTCCACATGTGCACCGTGGAAGCCGCGCCG |
| | GGCGTGGACCTCGACGGCGTGGAGACCTACCTCGGCCGCCCCTACAGGAACTTCTC |
| | CCACGTCGCCTTCATCAAGTCGTATCTCAGTCGCGTGGTCAGCCCCAACGGCTGGG |
| | TCGCGTGGAACAAGAACAAGGTCGTCGAGGATACCACCCGGACCATCTTATACCTG |
| | GAATACGGCAACGACGGSGCCGGTGCCGACACAGCCGGCCGYGTCAAGTGGCCGGG |
| | CTTCCGCGtCCTCAACACCGACGACGAGGCGATCGCGTACACGGCGGACACGTTCA |
| | TCACCGCGAGCAAGTGGGTCCCTGAGCCTATCCAGTACGTCCACACCCTCGGCACG |
| | GCGCCGCCGCCGCGCGCCTGA (SEQ ID NO: 7) |
| Tcb1- | GGCGCGGAGACGAGGCAGAAGCTGCCTGCTGGCAGTGGTAACGACGACGACCACGC |
| female | AGCCGTTCTCAGCCGCCTGTCCAACGTCATTGATCCGCCGGGGAGCTGGCCTCCGC |
| Mature | GTGCGGATGCTGTCGTGGCGAAGCGGTGCCGCGGCGTCGCTGCTCCGCCGCCTTGC |
| (na) | TACACCAGCATCCAGGCAGCCGTGGATCACGCACCAGCACCACAAGAAGCCGAGGA |
| | GGTGGAGGACAAGTACGTCGTGCATGTGCTCGCTGGCGTCTACGACGAGACCGTCA |
| | ACATAACAAGAAGGAACGTGATGCTGATCGGCGATGGGGTCGGTGCCACCGTCATC |
| | ACGGGGAACAAGAGTAATGCAACAGGCGTCCACATGGACATGACGGCGACAGTGAA |
| | TGCCTTGGGTCACGGCTTCATAGCACAGAACCTGACAATCAGAAACACGGCAGGGC |
| | CGGAAGGCAGGCAGGCCGTGGCGCTAAGGTCAAATTCGAACAAGTCGGTCGTCTAC |
| | TGGTGCAGCATTGAAGGTCATGAGGACACCTTGTACGTGGAGAACGGGATCCAGTT |
| | CTACCTGCAGACCTCGATCTGGGGCACCGTGGACTTTGTGTTTGGCAATGCCCAGG |
| | CCATGTTCCAGAGCTGCGCGCTGCTGGTGCGCCGCCCACCGAAAGGCAAGCACAAT |
| | GTGCTGACGGCCCAGGGCTGCAACAACGCAAGCCGCGAGTCCGGCTTCTCGTTCCA |
| | CATGTGCACCGTGGAAGCCGCGCCGGGCGTGGACCTCGACGGCGTGGAGACCTACC |
| | TCGGCCGCCCCTACAGGAACTTCTCCCACGTCGCCTTCATCAAGTCGTATCTCAGT |
| | CGCGTGGTCAGCCCCAACGGCTGGGTCGCGTGGAACAAGAACAAGGTCGTCGAGGA |
| | TACCACCCGGACCATCTTATACCTGGAATACGGCAACGACGGSGCCGGTGCCGACA |
| | CAGCCGGCCGYGTCAAGTGGCCGGGCTTCCGCGtCCTCAACACCGACGACGAGGCG |
| | ATCGCGTACACGGCGGACACGTTCATCACCGCGAGCAAGTGGGTCCCTGAGCCTAT |
| | CCAGTACGTCCACACCCTCGGCACGGCGCCGCCGCCGCGCGCCTGA |
| | (SEQ ID NO: 8) |
| Tcb1-male | ATAAATCCATTTTTAAAATTAAAATAATTCCGGAAAAGGCTGAAATCGTTTTTAAT |
| Promoter | GTCCAAAAAATATCTCAAAGGCTCCGAAAATTCTAGGAAAAAAATTCCCAGGCATG |
| (na) | ATTTGGCACACACTGAACTCAAAAAACATATTTAGAACTTTTGAAAATGTTGGAAC |
| | TACCTCAAATGAATAGATTTTGGTTCTCGGAGAGTAGAAAAATATTCAGAAAAATA |
| | TGAAAAATTTTCGGAAACTTATACAAGATATATTAACATGTTTCAAACACTTCTTG |
| | CACTTAGAAACACTATGCAACAACATGATGCAACATCCAAAGAACTTCTAGGGCTC |
| | ACGCCAACATAGAACAAAATAACTCTCCATTATTTTAGAAAGGGGAAAAGTAGAGA |
| | AAGGAGAGAGTAACAACTGAATTTTGAGTATAGAGCAAAGAAATTTTTGTATTCCA |
| | AAATTCAGGGTGTTACACGGGGCAGCAAGCCGGTGGCGGCGCGTCGGCTGTAGAGT |
| | AGGGGGGCAGAGCGTTATTGGCGGTGTCGTAAAGCTAGTGCATAGAAAATGGGCGA |
| | GCGTGAGCGCGTGTGGATGAAAAGTTGGGCACGATCCTCATAATGCATTAAACCTG |
| | TCGACCAAGATCAAGGGCCGACAAAAGTGATCAATCCCCGCATGTGTGTGAGATAG |
| | TTTGCCAGGCCCACACTAATCCCTGTCATCTAGGCTTCATGGCTGACATGAGTTAT |
| | TTATTCTCATGAGTAAGGAGGCCAACGAGAGTTATTTAATCTTCATCGGCTGACCT |
| | CTTGGCCGACAGTAATTAACTTAACTTTTGCCAGCGTCTTTACGACTGACGTTTAT |
| | TAGTTTGTCTCTGTCGACATTTTAAAAGGCCGACATGGATTATTTTGCCCGACGAG |
| | AGTTACCCTGATTCCTGTTATGTGTGTATAATTAAAGCTACACGTACTTTGTAAAA |
| | AACAAAAATGAATAGTAGTTTAGGAGTGCGGGGGTATAATGCATGGACCGTATTAG |
| | TAGCTTTGTAACATACTCCTATTTCAGGATATAGGACAGCTAGCTAGAGCAATAGC |
| | AGAGCTAGGTTTACGGGTAACCTTGCTGGAAATGTGGAGCAGTATGTTCGCTTAAT |
| | TTTCATAGCACATGTTCTGAATCTTTCTACAAAAAAATTCCTATACTAAATCGAGT |
| | TGGAGAATATATATTTGCAAAACCCACCCACCCAACCAAATTACCCCCACATCCAT |
| | AACTTCCACTAAATCCACCAAACAAATTACACCCAAACGTAACACACCAATAAAAC |
| | CAACGATTCATATTGGTTTGAGGTCTTGTACTTATCGAGTATGGTTTCTAACAGCT |
| | TCAGTAGGATTTATATGAAAAGTTTTAGTAAATTCATTTTGTTGAGGAATACAGAC |
| | TCGTTAATTTTCCATGCATTAATCAAAGGAACGTACATGCATTCATCAGAATTCAA |
| | CTTCTCAAATTAGGTTGTTGGACTCAGCTAATACCTAACAGCCTTCCATCAACAAC |
| | TAAAACAAAGGAACACCACTCCTTGCCATGTTCAATCACACACCTCTCATTCTCGA |
| | TAGTCCCAGGCTAGGTATCTATAAATAATGAACAATGAGGAAGGGAGGAATAAGTA |
| | GATTGAGAAATACTAAGAAGAAGATAACATCTCGACC (SEQ ID NO: 9) |
| Tcb1-female | CGGTGCAGGCCAGCCCGCCCATGCATCCGACGATCCAGTGCTCGACCGAGATGCAG |
| Promoter | CAGCAAGATATGGTTGGGTATAGCTAGAGCGCAGTTGGCCGGCCAGAACAATGCTA |
| (na) | GGCGAGAGAGGGAAGTGGTGGTGATAGGAATGTGAGAGGAGAGGGATTGTTATCTG |
| | TTAGGTCTTATGAATATGGAGGACCGGCCGGCAGCAAACAAAAAAGTGAACACTCA |
| | TCATAGCTGCTGTTGGGCAGGGATTCTTTTCCATCTCGCGTCCTCTGGTTGAGAGA |
| | TATATATATGTCTGCTGCTCTCGCTATATAGAGGGCCGGCCTGCCCTGCTAGCTAG |
| | CTAGCTAGCTAGTTCAAGTTCTAGGCACGGATCGATCAACATTAATTTAATTACTT |
| | AATTATTAGTTCCCGACGAAATTAATATATGTTAAACAAAGACTTTAATTTTTATT |
| | ATATCCTTATATATATATATATATATATATATATTATATAGTTATTATCAGTTTTA |
| | ATTTAATCCAGAAAACCAGTATGATGTATGTGATGACATGTATATGTGCATGTGTG |
| | TATTACTACTCGCATAAGCAGTAAACAACAGTAAAACATGCACAATCACTAAGAAC |
| | ATAATGTACCCTGCGGAGGTAGCGATACTCAAGACATCGTTGGCTCTATTCACACG |
| | AGACATTTCGTGTGTATGTTCGATTTAGTCGTATGCAGTCGATTCTGGTATATGTA |
| | CGCAGTGCGGTCCCTCGAACGGCATCGACGACGAAGAACCGGATCATCGTTGGTCG |
| | AGAGGATGGAGCGAGCAGTTGCGAGTACGCTCCCCAAAAACCTGATCGCCCGCACA |
| | CCCGTGCAAGTGTAGCTCTACGGACGTCGATTTCGGAGGCCTACTCTCCCACTCTC |
| | TCTGTGCTCGCGGACGGTGGGACGGTAACGGGCTGTGTCACTACACGACGGTTGAT |
| | CTTTAGCGACCCTTATTAGGTACCAATTGTTGGTTGCTAAAGGTTAGGGACCGACG |
| | GTCTGTCGCTAAATCCTTTTGTAGCAACGGTCGGTTGGTCGCTAAAAGTTTAGAAA |
| | TTTAACGACTTATAGTTGGTCGCTATAGATGTCGTCGGGGATTAGAGTCCGTGACC |
| | CAGCGTCTCAGGACCCAGGCTCCCAGTCCCAGCCTCTTCTATCCGGCATCCAAGGA |
| | GCAGCAAATCCAGAGACCTCGACTCCTCCTAACCCTAGCCGCCAGGCTCCAGTGCT |
| | CCACCGCTCCAGCAGATCCCGAAAGCAGCGAGGAAACACAGGGCGACCGAGGAATG |
| | TCTGGACTCTGGATCCGCCGGTCCGCCACACCACCCCTCATCTCCGAGTCTCCGAC |
| | CTGAGCGCGCCGCCCCGCCCCGCCCCGCCTCTCCCTCGGGCGCTGTCCGCGCCTCC |
| | GTGCCTCTGCGGTCCGCGTTCGGGCGTCCGCCCAGGGCCTAGGCAGTTGTTTCCTG |
| | CTCGCTTGCTGCCGTCCGCCAGGCCGCCTAGGCAGACATCACACTACTTCTACAGT |
| | TCTACGACCAGCGCCCAGCGGTCCAGTCCAGGTTAGTGCTTTTGCCATCATTTGGT |
| | CCAAATCTCTCTTTTCGTTATTTTTACGTACCCTAGTGTTGTTCCACAACGAAATG |
| | CCAAAATAATTCTTGTTGATTTTTCTATTATTGGTGTTAGTTCAATTCCGCTACAC |
| | ATTCAATAAAAATGCCAAGCTTTTTGTGACATATGCAGAAGAACCTTATTTACAAA |
| | CTGCTTGATGAACATTGTTCATTTTGATGATTAGATAAATTTTGTTTTTCAGTATA |
| | TCTGTACTCATTCTATTGTAAACAATTCATTCTAGATATGTAACAAAATGGCATGT |
| | TTATATCTTTGATTCATGATGCATTTCTTTCATACACCATGCTGTAAGAGTAGAAT |
| | TGATGATTCTTTCATCGCATTCAATAATCTCACAAGATTGTCTTCCATTTTCATAG |
| | TTGGTACCAGTAGTGACTGAGCTCATTGTTGCCCAGTTGATGTACCTTGAATGGAT |
| | GAACAGTAAAAAACCTGTTTATATATACATCAACTCAACTGGAATGGCTCGCGATG |
| | ATCGTGAACCGGTAAGCAAGATTATTCAATTGGTGACTTCAGTCAAGTATTTCCAG |
| | TATTGGGGTCAGTATCGGTAGAATTTGGCTTATTCATGTTCTTCTAATTAGGGTAG |
| | ATTATAAGGTCTCCTTAGCATTTGTATCATGTCGTCATGGTTGTTATATAGTCAAA |
| | TAGTTCTTTAATTGATTGTCTGTATTTGTCAGGTCGGGATGGAGAGCGAAGGTTTT |
| | GCAATCTATGATGCAATGATGAGTATGAAAACTGAGGTGCTATGCCTTACATTTAC |
| | TATCTGCAGTTCAATAGTGTAATGTTGATCATTTGGTACAGATTGATGCTGATTAT |
| | TTACTGAAACATGAACATCTATTTTATGCAAAAAAGTTCTCAGTGGACATAATTTA |
| | TTTTTAAAAGAAGAATTTTACTGTGTATGCAATCTCGATCTGCCTAGTTCTGTAGA |
| | GATTACTAATGATTCAATGAATGGCTTGTCGATCATTTGTTTGAACTGGATGGAGC |
| | GTGTAAAAAACATGATCAGCATTGGAGGAACTAGGTGGCAATGGCATCATAACCAA |
| | GAAGAAAATTAGGCTTCCAAATTTGAGATGAAGACGATGACTCAAACTTGAGCCAT |
| | GGAGGTGTTCACCAGCAGCCCCACCAACTTAGTTACACCGAGTTCCGATGGAGTGT |
| | GTAAAAGCAAACTTCTTTGTGTATTTATTTGTTCAATGAAAGAGTGGCCATAAATA |
| | CAAACTTTGCACTGTATGCTGATATTCTGAAGTGATCAGCACCCTGTTTTCATCAC |
| | CATAAGATTAAGATGGGATGTTTGTATCAAATAAATCTACAGTTTAGCCAAACTTT |
| | GCACTAGCGCTGATATCAATAACTTGTTAGGTCCCTGTTTCATCAACATAACATTA |
| | GGACCGGATGTTTGTACAAATTGAATTCAGTGTCCAAACTGTGAATTGTTAAAATT |
| | CATCATTTTTTCTAAATTTATGGACATCATGATGAGGTTACTATTTGCCATGCAGA |
| | TCCACACACTTTGTATAGGAGCTGCAGCAGGCCATGCATGTCTTGTGCTTGCAGCT |
| | GGAAAAAAAGGCAAACATTATATGTTCCCTCATGCCAAAGGTAGTAACGTGATAAC |
| | TTCCGCACCTGTTTTCTTTCCCACGGGAAATACTCACTTTTGCTCATCATTTCCTG |
| | CTGATTCTGGATCATACTATGTCAGCTATGATTCAGCAACCACGTATTCCTTCTTA |
| | TGGGATGATGCAAGCATCAGATGTTGTTATTCGTGCAAAGGAGGTACGGGCCATGG |
| | TTTTTTGAGTTGCTTATATTTTGAGTGTTATTTCGTATTCTAATGTATGCTTATAT |
| | TTTTATTTGCAAATGTAGCAACAATATACTATTTTGTTGTACTTTTTCTAGGGTGT |
| | TCTAGACAAAGAAAGCTTGTACCTTAAAACAGGAGGATACAAGAAGCTGGAGTGGT |
| | TGTCTGAAGGCTTTTGAACTGCTTGAAGTATTAGTGTAGCTTTGGTGCTTTTGTGA |
| | CTGATCAGCTCTGGAGCAGCTAGAGTGATGTAGTTGTGGGCTTTTGAACTGCTTCA |
| | AGCTGAAGTGATGTCGCTGTGGTGCTTTTGTGACTGATCATCTCTAGAGCTTTTGT |
| | GAATGATCTAGCTAGCTATCTGTAAATGATCTAGTTGTGAATGATATTATAATGGT |
| | GATGCTTTTGTGAATATATAATTGTGGTGCTTTTGTGTTTATGTGATGGATCTATG |
| | ACTATGGTATACTGATTAACTGTGTATGTCTTTATGATTTGTGTATAAAATTCTGT |
| | GATTTGTGATGCTTAATTAAATGTATGTTATTATTAATAACAACAGTAAAAAGGGC |
| | GACTTTCTGTTGGTTGCTAAATGTATAGTATGCCGACTTACGGTTGGTTGCTAAAT |
| | CTATAGTACAGCAACCCACGGTTAGTCGCTAAATATACAATATTAGCAACGGTCGG |
| | TCGGTCGCTAAAAAGATGTCGGTCGCTAAAGCCTTTAGCGACGGCACTTACAACGA |
| | CCATCCTTATGGGTCGCTAAAAGTTTTTAGCGACCAACCGTAGGTCGCTGAAGGCC |
| | GTTTTAGCAACCAACCGTAGGTCACTGTAAGTGAACCGTCGTGTAGTGTGTGCAAC |
| | GCGTATGAGAGACTGTTCGCATAACTGGAAGAGCGTCCACCTCCTCCGTATTTATA |
| | CACGCGCATTGGGAGGGGAACAGACTATAACAGTCGCCATCAGAGTCAGAAACTGA |
| | CAGCCATTACAGCCAGCCAGAAACCGATGTACCATAAGAGACGCCTGTTACTAGCC |
| | ATAAGACAGGAAACAACTGATCATTACTCCAGGCAAAATGCGCAATCGTTAGAAAG |
| | AAATATTTGGACTAAGGTCCGATTTACCACGAGCCACGGCCCGGCTCGGCCGGCGG |
| | CGCCTGCGCACGATTGGGCAGTCCTCAATCCTTTTCTCAACTTCTCAACAGATACA |
| | CCAATGGTCCACCTATCTAAGTTAATTGATTTGTCCTTTGAACTTCCGGTATGGTA |
| | CTAAAATATTAGTGCACCATAGCATTAAAGTGGACCTTTAACATTGACTATTATTG |
| | AATATTAATTTGGGCCAAGCCCACATTAATCCAACAATCCCCATCAAGTGCTAAGT |
| | CACACTAAAAATGCTCTTATCATCTCAAACGTTTGATATACATGTGTTTCGATGGA |
| | GACTGTTAAGTTGAACATCCACCTAGAACCTAGACTACACTTAACCACAACTGCAC |
| | CTAGAACTAATGAAGATATTTCAGTAATAACCAATTGGGTGAAACTATATATATAT |
| | ATATATATATATATATATATATATATATATACCATCGCTATTTGTCAGTCTACCCA |
| | ACCGATAATTATTGAGCGGACTTCAGTAATAATCGAGAACTAATGAACATCGTTCA |
| | GTAATAACCAATTAGGCTCCTCATCCACCTCTTTCGTAACCAATCATCGAAATTTT |
| | AGCACAATTTTCCTTGTGTAGAACTCCATCAGACGGTCTATCTTTCCACATTCATT |
| | CTCAACAAATTTTCCTAGCAGCCTCATCCTTCTTGTGCCTTTGTTAATACAACCTA |
| | AAGGCCGGTATAAAATAACCAAACATGACAAATACATAAAACAAATAAATCACAAG |
| | AATAAATTTCTCCATGACATCTGCATTCAATCTGCTAGCTGAACATTCTATCGTGA |
| | TTAGTGGGTTTGCTAAAGCACCAGGATAGGCAGTAGAACGAAGAGCCTCTAAAAGG |
| | AAAGTTTCAGGGTCACTTGATGATTTCGCAAGCTCTGCCTTTAGCCTTAAAATCTA |
| | AAAAGAAAACATGACACTATGTCAGCAAAGAACATGATCTTTTAATCTAAAACGAC |
| | ATGTAGGGGATGACATCAAGATCAAATACCTATTCATTGAGCTCCCAATTAAGAGA |
| | GCAGGGTTGCGTACACAATCAATAAGCACCTCAACCTGGAATCCCAGGCATATAAG |
| | TCTTTTGGGCTCCACAAGTGTAGCTCATCATCTCACGACAAGTAGATGCTTTGGAA |
| | CAACCACCTATTGCCTCAATTTTACGCACACCACACAATTCGCTCTTGTTGGTAGT |
| | GAATGCAAAGTCCATTGTATCTTCAATAGCTGACCGGCACCTGCTCAGAACCATAA |
| | TCAACATAAACTCCAACAAAATCAGATGGTCCCTGAATAGAAAATATGAACACCCT |
| | ATTATCATCAGCTACTGCTGAGGGATTGGATGGAATGTGTAACCATATAAAAATAA |
| | AGACAACATACTGCAGATGTCTCAGAGGCTATTTTAGAACATACAATGTTGAGTTG |
| | CTGCTCTCCACTAATATATGTTGTATTAACACTGAGGAGGGTGCAACAAATGCTCA |
| | TAAAAGTAATATCAAACTGTGAATTGGAGTTACTATTATGAAAGACATTTGTTAGG |
| | CACCACCACACGACAATGTATGCACATGTCTCTTTAAAACAGAAAGAATAGAAGTG |
| | TAAGAAATAACGTATAGACAATTTATTTGGTAAAAAAAAGGCCAGAAGAGTAGGAT |
| | GGATCACGACGGGCATCCAAGTTGTTTCATCTCTCTGTGAAGAACCTTAGATGTAT |
| | TGCACCCCTTGCAGCAGTTCTAAAAAGTTATTTAACCATATGATAAAAGTAGAAAT |
| | ATCTACGGTTGGATGGACAACATCCAAGAATATATAAAGTAATAAAAATTTGCATT |
| | TTGAATTTTAAAATTTTGAATGTAATTTTTGAGAGCAGAGATGATTTCAAATAAAA |
| | AAGTTATCAACTAAAAGGTTTCATAACTTTTCGAGATCCACAACTTTTATGTTGGT |
| | GGTTTTATCATCCAAAATCGTTTGGAAAACTCAAAAAATTAAGGATATAAATGATT |
| | TCTAGTGGCGGTTTTTTAAGAAAACCGCCACTAGAAATTATGGTGGTTTTCTTAAG |
| | GAACTACCACTAAAAAATAGCACTAACGATCAGTCTTTAAAAAAAATACACCCCAC |
| | TATGCAGGCTTTGGTGTAAAGCAATCACCGTGCATCGTCGTCCTCAACACACTCGG |
| | AAAAACTATATATATAGAATCCATAAATAAGAGAGGAAAGAACCCGTGCAGAAATC |
| | TTTTTAGTTTACTGAATCTTTCTGGGAAACTTTTTCTTTATTAAATCGTCTTGGGG |
| | GAACATTTTCCTTTACTAAACTACTAGGAGGCTGACATGAAGGGCCCTTCTTCCCT |
| | GATCCATTATTCTTGCCCCGCGCCGGCTTCTCGTCATGGGCACACCGCACATGCAG |
| | GCTTCTGGCTCGATCGGGCAGGGAGGGCCGGGCCTATATATATGGGATGCTGCCGG |
| | CGGTGTTAGCCAGCGAAGACGCGCACAAAGGTAAGTGTGTAAGGTTAGGAGCCAGT |
| | GTACCATCACATCAGATCAGATCGATCGCAGCTTATAGGGG (SEQ ID NO: 10) |
| GA1-female | TAAAGCAATCACCGTGCATCGTCGTCCTCAACACACTCGGAGAAACTATATATATA |
| Promoter | GAATCCATAAATAAGAGAGGAAAGAACCCGCGCAGAAATCTTTTTAGTTTACTGAA |
| (na) | TCTTTCTGGGAAACTTTTTCTTTATTAAATCGTTTTGGGGGAACATTTTCCTTTAC |
| | TAAACTACTAGGAGGCTGACATGAAGGGCCCTTCTTCCCTGATCCATTATTCTTGC |
| | CCCGCGCCGGCTTCTCGTCATGGGCACACGGCACATGCAGGCTTCTGGCTCGACCG |
| | GGCCTATATATATGGATGCTGCCGGTGTTAGCCAGCGAAGACGCGCACAAAGGTAA |
| | GTGTGTAAGGTTAGGAGCCAGTGTACCATCACATCAGATCAGATCGATCGCAGCTT |
| | ATAGGGG (SEQ ID NO: 12) |
| GA1-male | MMMSKQMLVLSLLLVLFELGSLPITSCKKVFFNLWVINQPANATQDAGCAKKDDAL |
| Full- | SSADTIKVWNYIDPASQLRPEDGGYTTISESIANIPEDNAKRYLLILKPGWFREKL |
| Length | LLGRSKPFITIMSEDPMNPAVIVWNDTATTMGKDGKPLGVDGSSTMAIESDYFVAY |
| (aa) | NVVFKNDAPLPKLGEKKGEAPALRVMGTKATFYNCTIEGGQGALYDQTGLHYFKAC |
| | AIKGTIDFIFGSAKSFYEECKIVSVLKEALVLPLAPPEQDRSRNPIEIAPGKSGLA |
| | FKTCTIEGEGEKTYLGRVGTPVIYSYTNIGKEIVGIISDGRDVQTVERGYYCATFR |
| | CYGPGMSPMVTSTLTYVEAIPFLGIHYISGESWIPSLPPAEE (SEQ ID NO: 13) |
| GA1-male | TTSCKKVFFNLWVTNQPANATQDAGCAKKDDALSSADTIKVWNYIDPASQLRPEDG |
| Mature | GYTTISESIANIPEDNAKRYLLILKPGWFREKLLLGRSKPFITIMSEDPMNPAVIV |
| (aa) | WNDTATTMGKDGKPLGVDGSSTMAIESDYFVAYNVVFKNDAPLPKLGEKKGEAPAL |
| | RVMGTKATFYNCTIEGGQGALYDQTGLHYFKACAIKGTIDFIFGSAKSFYEECKIV |
| | SVLKEALVLPLAPPEQDRSRNPIEIAPGKSGLAFKTCTIEGEGEKTYLGRVGTPVI |
| | YSYTNIGKEIVGIISDGRDVQTVERGYYCATFRCYGPGMSPMVTSTLTYVEAIPFL |
| | GIHYISGESWIPSLPPAEE (SEQ ID NO: 14) |
| GA1-male | ATGATGATGAGTAAACAAATGCTCGTCTTGTCCCTGCTCCTAGTGTTGTTCGAGCT |
| Full-Length | TGGATCTCTGCCGACGACATCGTGCAAAAAGGTCTTTTTCAACTTATGGGTGACAA |
| (na) | ACCAGCCAGCTAATGCCACCCAAGATGCGGGGTGTGCTAAGAAAGATGATGCGCTC |
| | TCCTCCGCCGACACCATTAAGGTATGGAATTACATCGACCCTGCCTCTCAATTGAG |
| | ACCTGAAGATGGCGGTTACACGACCATTAGCGAGTCCATCGCCAACATCCCTGAGG |
| | ACAACGCCAAACGCTACCTCCTTATCCTCAAACCTGGTGTTGTGTTCCGCGAGAAG |
| | CTGTTACTCGGTAGAAGCAAGCCTTTCATCACCATAATGTCCGAGGACCCCATGAA |
| | CCCTGCTGTTATCGTCTGGAATGACACTGCCACCACCATGGGCAAGGACGGCAAGC |
| | CCCTTGGTGTGGATGGAAGCAGTACCATGGCCATAGAGTCCGACTATTTTGTCGCC |
| | TACAACGTTGTCTTCAAGAACGACGCGCCGCTACCAAAGCTAGGGGAAAAGAAAGG |
| | TGAGGCACCAGCACTGCGAGTGATGGGAACAAAGGCAACCTTCTACAATTGCACCA |
| | TCGAAGGCGGCCAGGGTGCTCTGTACGACCAGACGGGTCTGCACTACTTCAAGGCT |
| | TGTGCCATCAAGGGAACCATCGACTTCATCTTCGGATCTGCCAAGTCATTTTATGA |
| | GGAATGCAAAATCGTTTCGGTGTTGAAGGAGGCATTGGTATTGCCATTGGCACCAC |
| | CGGAGCAGGACCGCTCTAGAAATCCCATCGAAATCGCCCCAGGCAAGAGCGGGTTG |
| | GCATTCAAGACTTGCACAATCGAGGGGGAAGGAGAAAAAATTTACTTGGGTAGGGT |
| | GGGCACGCCTGTGATATACTCCTACACTAATATAGGTAAGGAGATTGTAGGCATAA |
| | TATCTGATGGTCGGGACGTCCAGACAGTCGAAAGGGGGTACTACTGCGCCACTTTT |
| | AGGTGTTACGGGCCTGGGATGTCTCCAATGGTAACCTCAACTCTGACCTATGTCGA |
| | GGCAATACCCTTTCTCGGGATACACTACATCTCGGGGGAGTCATGGATCCCGTCCC |
| | TACCACCCGCTGAAGAATAA (SEQ ID NO: 15) |
| GA1-male | GCCGACGACATCGTGCAAAAAGGTCTTTTTCAACTTATGGGTGACAAACCAGCCAG |
| Mature | CTAATGCCACCCAAGATGCGGGGTGTGCTAAGAAAGATGATGCGCTCTCCTCCGCC |
| (na) | GACACCATTAAGGTATGGAATTACATCGACCCTGCCTCTCAATTGAGACCTGAAGA |
| | TGGCGGTTACACGACCATTAGCGAGTCCATCGCCAACATCCCTGAGGACAACGCCA |
| | AACGCTACCTCCTTATCCTCAAACCTGGTGTTGTGTTCCGCGAGAAGCTGTTACTC |
| | GGTAGAAGCAAGCCTTTCATCACCATAATGTCCGAGGACCCCATGAACCCTGCTGT |
| | TATCGTCTGGAATGACACTGCCACCACCATGGGCAAGGACGGCAAGCCCCTTGGTG |
| | TGGATGGAAGCAGTACCATGGCCATAGAGTCCGACTATTTTGTCGCCTACAACGTT |
| | GTCTTCAAGAACGACGCGCCGCTACCAAAGCTAGGGGAAAAGAAAGGTGAGGCACC |
| | AGCACTGCGAGTGATGGGAACAAAGGCAACCTTCTACAATTGCACCATCGAAGGCG |
| | GCCAGGGTGCTCTGTACGACCAGACGGGTCTGCACTACTTCAAGGCTTGTGCCATC |
| | AAGGGAACCATCGACTTCATCTTCGGATCTGCCAAGTCATTTTATGAGGAATGCAA |
| | AATCGTTTCGGTGTTGAAGGAGGCATTGGTATTGCCATTGGCACCACCGGAGCAGG |
| | ACCGCTCTAGAAATCCCATCGAAATCGCCCCAGGCAAGAGCGGGTTGGCATTCAAG |
| | ACTTGCACAATCGAGGGGGAAGGAGAAAAAATTTACTTGGGTAGGGTGGGCACGCC |
| | TGTGATATACTCCTACACTAATATAGGTAAGGAGATTGTAGGCATAATATCTGATG |
| | GTCGGGACGTCCAGACAGTCGAAAGGGGGTACTACTGCGCCACTTTTAGGTGTTAC |
| | GGGCCTGGGATGTCTCCAATGGTAACCTCAACTCTGACCTATGTCGAGGCAATACC |
| | CTTTCTCGGGATACACTACATCTCGGGGGAGTCATGGATCCCGTCCCTACCACCCG |
| | CTGAAGAATAA (SEQ ID NO: 16) |
| GA1-female | MVGGVRRCGLGLAMAVALLLAALVVVASGGAEMRQKLPAGSGNDDDHAAVLSRLSN |
| Full-Length | VIDPPGSWPPRADAVVAKRCRGVAAPPPCYTSIQAAVDHAPAPQEAEEVEDKYVVH |
| (aa) | VLAGVYDETVNITRRNVMLIGDGVGATVITGNKSNATGVHMDMTATVNALGHGFIA |
| | QNLTIRNTAGPEGRQAVALRSNSNKSVVYWCSIEGHEDTLYVENGIQFYLQTSIWG |
| | TVDFVFGNAQAMFQSCALLVRRPPKGKHNVLTAQGCNNASRESGFSFHMCTVEAAP |
| | GVDLDGVETYLGRPYRNFSHVAFIKSYLSRVVSPNGWVAWNKNKVVEDTTRTILYL |
| | EYGNDGAGADTAGRVKWPGFRVLNTDDEATAYTADTFINASKWVPEPIQYVHTLGT |
| | APPPRA (SEQ ID NO: 17) |
| GA1-female | GAEMRQKLPAGSGNDDDHAAVLSRLSNVIDPPGSWPPRADAVVAKRCRGVAAPPPC |
| Mature | YTSIQAAVDHAPAPQEAEEVEDKYVVHVLAGVYDETVNITRRNVMLIGDGVGATVI |
| (aa) | TGNKSNATGVHMDMTATVNALGHGFIAQNLTIRNTAGPEGRQAVALRSNSNKSVVY |
| | WCSIEGHEDTLYVENGIQFYLQTSIWGTVDFVFGNAQAMFQSCALLVRRPPKGKHN |
| | VLTAQGCNNASRESGFSFHMCTVEAAPGVDLDGVETYLGRPYRNFSHVAFIKSYLS |
| | RVVSPNGWVAWNKNKVVEDTTRTILYLEYGNDGAGADTAGRVKWPGFRVLNTDDEA |
| | IAYTADIFINASKWVPEPIQYVHILGTAPPPRA (SEQ ID NO: 18) |
| GA1-female | ATGGTAGGCGGCGTGAGGAGGTGCGGCCTGGGCCTGGCCATGGCGGTGGCCCTGCT |
| Full- | GCTCGCTGCGCTGGTTGTCGTTGCAAGCGGCGGCGCGGAGATGAGGCAGAAGCTGC |
| Length | CTGCTGGCAGTGGTAACGACGACGACCACGCAGCCGTTCTCAGCCGCCTGTCCAAC |
| (na) | GTCATTGATCCGCCGGGGAGCTGGCCTCCGCGTGCGGATGCTGTCGTGGCGAAGCG |
| | GTGCGGCGGCGTCGCTGCTCCGCCGCCTTGCTACACCAGCATCCAGGCAGCCCTGA |
| | AAGCCGCATCAGCACCACAAGAAGCCGAGGAGGTGGAGGACAAGTACGTCGTGCAT |
| | GTGCTTGCTGGCGTCTACGACGAGACCGTCAACATAACAAGAAGGAACGTGATGCT |
| | GATCGGCGATGGGGTCGGTGCCACCGTCATCACGGGGAACAAGAGCAATGCAACAG |
| | GCGTCCACATGGACATGACGGCGACAGTGAGTGAGTAACTCTGGCGATCGATATTT |
| | CACTGCAGTTGAGCTGTCATGCATGCATACTGTAAATCAGAACAGAACTATTAATT |
| | TGCTGCTGCTGCTGCAGATGCCTTGGGTCACGGCTTCATAGCACAGAACCTGACAA |
| | TCAGAAACACGGCAGGGCCGGACGGCAGGCAGGCCGTGGCGCTAAGGTCAAATTCG |
| | AACAAGTCGGTCGTCTACTGTTGCAGCATTGAAGGTCATGAGGACACCTTGTACGT |
| | GGAGAACGGGATCCAGTTCTACCTGCAGACCTCGATCTGGGGCACCGTGGACTTTG |
| | TGTTTGGCAATGCCCAGGCCATGTTCCAGAGCTGCGCGCTGCTGGTGCGCCGCCCA |
| | CCGAAAGGCAAGCACAATGTGCTGACGGCCCAGGGCTGCAACAACGCAAGCCGCGA |
| | GTCCGGCTTCTCGTTCCACATGTGCACCGTGGAAGCCGCGCCGGGCGTGGACCTCG |
| | ACGGCGTGGAGACCTACCTCGGCCGCCCCTACAGGAACTTCTCCCACGTCGCCTTC |
| | ATCAAGTCGTATCTCAGTCGCGTGGTCAGCCCCAACGGCTGGGTCGCGTGGAACAA |
| | GAACAAGGTCGTCGACGATACCACCCGGACCATCTTATACCTGGAATACGGCAACG |
| | ACGGCGCCGGTGCCGACACAGCCGGCCGCGTCAAGTGGCCGGGCTTCCGCGTCCTC |
| | AACACCGACGACGAGGCGATCGCGTACACGGCGGACACGTTCATCAACGCGAGCAA |
| | GTGGGTCCCTGAGCCTATCCAGTACGTCCACACCCTCGGCACGGCGCCGCCGCCGC |
| | GCGCCTGACGATGTTCGGCTCGGCGCGCTTAAGTTAGTACTGTACTACGCAGGCTG |
| | CTGGCGTACTGTACTACGGTATTGTATGTCTCTAGTATCCAGCCATGCATCATTGC |
| | CGAGATGATTTAAGGCATATTGTGGCATGCATGACGCCGTATATTTCGCGTATGCA |
| | TGCATGTACCTGGACGGCTGGACCTCTACCTGTAGCTACCATTTCTCTACCGGTAT |
| | TGTACGTACTCTCTTTGTATGTGTGTCTATATTATATATGTATCCTCGATCTAAAT |
| | AAATGGATTACATCTATCATTATTTC (SEQ ID NO: 19) |
| GA1-female | GTTCTCAGCCGCCTGTCCAACGTCATTGATCCGCCGGGGAGCTGGCCTCCGCGTGC |
| Mature | GGATGCTGTCGTGGCGAAGCGGTGCGGCGGCGTCGCTGCTCCGCCGCCTTGCTACA |
| (na) | CCAGCATCCAGGCAGCCCTGAAAGCCGCATCAGCACCACAAGAAGCCGAGGAGGTG |
| | GAGGACAAGTACGTCGTGCATGTGCTTGCTGGCGTCTACGACGAGACCGTCAACAT |
| | AACAAGAAGGAACGTGATGCTGATCGGCGATGGGGTCGGTGCCACCGTCATCACGG |
| | GGAACAAGAGCAATGCAACAGGCGTCCACATGGACATGACGGCGACAGTGAATGCC |
| | TTGGGTCACGGCTTCATAGCACAGAACCTGACAATCAGAAACACGGCAGGGCCGGA |
| | CGGCAGGCAGGCCGTGGCGCTAAGGTCAAATTCGAACAAGTCGGTCGTCTACTGTT |
| | GCAGCATTGAAGGTCATGAGGACACCTTGTACGTGGAGAACGGGATCCAGTTCTAC |
| | GTTCCAGAGCTGCGCGCTGCTGGTGCGCCGCCCACCGAAAGGCAAGCACAATGTGC |
| | TGACGGCCCAGGGCTGCAACAACGCAAGCCGCGAGTCCGGCTTCTCGTTCCACATG |
| | TGCACCGTGGAAGCCGCGCCGGGCGTGGACCTCGACGGCGTGGAGACCTACCTCGG |
| | CCGCCCCTACAGGAACTTCTCCCACGTCGCCTTCATCAAGTCGTATCTCAGTCGCG |
| | TGGTCAGCCCCAACGGCTGGGTCGCGTGGAACAAGAACAAGGTCGTCGACGATACC |
| | ACCCGGACCATCTTATACCTGGAATACGGCAACGACGGCGCCGGTGCCGACACAGC |
| | CGGCCGCGTCAAGTGGCCGGGCTTCCGCGTCCTCAACACCGACGACGAGGCGATCG |
| | CGTACACGGCGGACACGTTCATCAACGCGAGCAAGTGGGTCCCTGAGCCTATCCAG |
| | TACGTCCACACCCTCGGCACGGCGCCGCCGCCGCGCGCC (SEQ ID NO: 20) |
| ZmPME 10-1 | DDNDGNMLSSVKVSTVCAFTRYPEKCEQSLKHVVSDTSSPEDVERDALNVALDEVS |
| Mature (aa) | TAFQRSAHIGKDDATEDLRALARVKPADVVRHVKDLRVWVSGIMTYVYTCADGFEK |
| | PELKEAMDKVLQNSTELSSNALAILTRLGDLMPGKAKDLQATLAGAVGHDRRLLGW |
| | QIGDAEEVTSGGRGLLDEIVGVANANRKLLSDTLDEITGMSHGANGRRLLSSLGSR |
| | ISSAQGDDVLARHQLLGVSPDDETDNAARRNLLSTELESIASTSAEANRQLLAAEE |
| | LPDELAGKRELLSRTLMGIDEAATEAKRQLDEATTENTMSGDHRVLTTGLIGTFDE |
| | IQDGRSGVPPSDFPKWLPATQRRLLQQTQKPNTVVAQDGSGDFKTITEAITAVPNT |
| | FEGREVIYVKAGTYKEYVTVPKNMANIFMYGDGPTQTVVTGDKSNAGGFATFASAT |
| | FSAEGNGFICKSMGFVNTAGPEGHQAVAMHVQGDKSVFYNCRFEGYQDTLYVHANR |
| | QFFRDCEVLGTVDFIFGNSAALFQNCLMTVRKPGDSQSNMVTAQGRTDPNMPTGIV |
| | LQGCRIVPEQALFPDRLQIATYLGRPWKEYARTVVMESTIGDLIRPEGWAEWMGDL |
| | GLKTLYYAEYANTGP (SEQ ID NO: 21) |
| ZmPME 10-1 | MANRVTVASVIAAVGIVAVIGTMATVTSADDNDGNMLSSVKVSTVCAFTRYPEKCE |
| full-length | QSLKHVVSDISSPEDVERDALNVALDEVSTAFQRSAHIGKDDATEDLRALARVKPA |
| (aa) | DVVRHVKDLRVWVSGIMTYVYTCADGFEKPELKEAMDKVLQNSTELSSNALAILTR |
| | LGDLMPGKAKDLQATLAGAVGHDRRLLGWQIGDAEEVISGGRGLLDEIVGVANANR |
| | KLLSDTLDEITGMSHGANGRRLLSSLGSRISSAQGDDVLARHQLLGVSPDDETDNA |
| | ARRNLLSTELESIASTSAEANRQLLAAEELPDELAGKRELLSRTLMGIDEAATEAK |
| | RQLDEATTENTMSGDHRVLTTGLIGTFDEIQDGRSGVPPSDFPKWLPATQRRLLQQ |
| | TQKPNIVVAQDGSGDEKTITEAITAVPNIFEGREVIYVKAGTYKEYVTVPKNMANI |
| | FMYGDGPTQTVVTGDKSNAGGFATFASATFSAEGNGFICKSMGFVNTAGPEGHQAV |
| | AMHVQGDKSVFYNCRFEGYQDTLYVHANRQFFRDCEVLGTVDFIFGNSAALFQNCL |
| | MTVRKPGDSQSNMVTAQGRTDPNMPTGIVLQGCRIVPEQALFPDRLQIATYLGRPW |
| | KEYARTVVMESTIGDLIRPEGWAEWMGDLGLKTLYYAEYANTGP |
| | (SEQ ID NO: 22) |
| GA1-male | ATGATGATGAGTAAACAAATGCTCGTCTTGTCCCTGCTCCTAGTGTTGTTCGAGCT |
| Full-Length | TGGATCTCTGCCGACGACATCGTGCAAAAAGGTCTTTTTCAACTTATGGGTGACAA |
| (na) | ACCAGCCAGCTAATGCCACCCAAGATGCGGGGTGTGCTAAGAAAGATGATGCGCTC |
| | TCCTCCGCCGACACCATTAAGGTATGGAATTACATCGACCCTGCCTCTCAATTGAG |
| | ACCTGAAGATGGCGGTTACACGACCATTAGCGAGTCCATCGCCAACATCCCTGAGG |
| | ACAACGCCAAACGCTACCTCCTTATCCTCAAACCTGGTGTTGTGTTCCGCGAGAAG |
| | CTGTTACTCGGTAGAAGCAAGCCTTTCATCACCATAATGTCCGAGGACCCCATGAA |
| | CCCTGCTGTTATCGTCTGGAATGACACTGCCACCACCATGGGCAAGGACGGCAAGC |
| | CCCTTGGTGTGGATGGAAGCAGTACCATGGCCATAGAGTCCGACTATTTTGTCGCC |
| | TACAACGTTGTCTTCAAGAACGACGCGCCGCTACCAAAGCTAGGGGAAAAGAAAGG |
| | TGAGGCACCAGCACTGCGAGTGATGGGAACAAAGGCAACCTTCTACAATTGCACCA |
| | TCGAAGGCGGCCAGGGTGCTCTGTACGACCAGACGGGTCTGCACTACTTCAAGGCT |
| | TGTGCCATCAAGGGAACCATCGACTTCATCTTCGGATCTGCCAAGTCATTTTATGA |
| | GGAATGCAAAATCGTTTCGGTGTTGAAGGAGGCATTGGTATTGCCATTGGCACCAC |
| | CGGAGCAGGACCGCTCTAGAAATCCCATCGAAATCGCCCCAGGCAAGAGCGGGTTG |
| | GCATTCAAGACTTGCACAATCGAGGGGGAAGGAGAAAAAATTTACTTGGGTAGGGT |
| | GGGCACGCCTGTGATATACTCCTACACTAATATAGGTAAGGAGATTGTAGGCATAA |
| | TATCTGATGGTCGGGACGTCCAGACAGTCGAAAGGGGGTACTACTGCGCCACTTTT |
| | AGGTGTTACGGGCCTGGGATGTCTCCAATGGTAACCTCAACTCTGACCTATGTCGA |
| | GGCAATACCCTTTCTCGGGATACACTACATCTCGGGGGAGTCATGGATCCCGTCCC |
| | TACCACCCGCTGAAGAATAA (SEQ ID NO: 23) |
| GA1-male | ACGACATCGTGCAAAAAGGTCTTTTTCAACTTATGGGTGACAAACCAGCCAGCTAA |
| Mature | TGCCACCCAAGATGCGGGGTGTGCTAAGAAAGATGATGCGCTCTCCTCCGCCGACA |
| (na) | CCATTAAGGTATGGAATTACATCGACCCTGCCTCTCAATTGAGACCTGAAGATGGC |
| | GGTTACACGACCATTAGCGAGTCCATCGCCAACATCCCTGAGGACAACGCCAAACG |
| | CTACCTCCTTATCCTCAAACCTGGTGTTGTGTTCCGCGAGAAGCTGTTACTCGGTA |
| | GAAGCAAGCCTTTCATCACCATAATGTCCGAGGACCCCATGAACCCTGCTGTTATC |
| | GTCTGGAATGACACTGCCACCACCATGGGCAAGGACGGCAAGCCCCTTGGTGTGGA |
| | TGGAAGCAGTACCATGGCCATAGAGTCCGACTATTTTGTCGCCTACAACGTTGTCT |
| | TCAAGAACGACGCGCCGCTACCAAAGCTAGGGGAAAAGAAAGGTGAGGCACCAGCA |
| | CTGCGAGTGATGGGAACAAAGGCAACCTTCTACAATTGCACCATCGAAGGCGGCCA |
| | GGGTGCTCTGTACGACCAGACGGGTCTGCACTACTTCAAGGCTTGTGCCATCAAGG |
| | GAACCATCGACTTCATCTTCGGATCTGCCAAGTCATTTTATGAGGAATGCAAAATC |
| | GTTTCGGTGTTGAAGGAGGCATTGGTATTGCCATTGGCACCACCGGAGCAGGACCG |
| | CTCTAGAAATCCCATCGAAATCGCCCCAGGCAAGAGCGGGTTGGCATTCAAGACTT |
| | GCACAATCGAGGGGGAAGGAGAAAAAATTTACTTGGGTAGGGTGGGCACGCCTGTG |
| | ATATACTCCTACACTAATATAGGTAAGGAGATTGTAGGCATAATATCTGATGGTCG |
| | GGACGTCCAGACAGTCGAAAGGGGGTACTACTGCGCCACTTTTAGGTGTTACGGGC |
| | CTGGGATGTCTCCAATGGTAACCTCAACTCTGACCTATGTCGAGGCAATACCCTTT |
| | CTCGGGATACACTACATCTCGGGGGAGTCATGGATCCCGTCCCTACCACCCGCTGA |
| | AGAATAA (SEQ ID NO: 24) |
| GA2-male | MESRRKNQQKTSYIVISIQLLAALLLLELVSLVPGASCEELPLDFWVSALRGVGAK |
| Full-Length | DVSTADGCTKKNKDTVLCSAQANTVTNFINPTNSEEQGYRTIGESIANIPDDSTKR |
| (aa) | YILILSGGTVYREKVLVSKSKPFVTIRSDDPINPAIIVWNDTAATLGKDSKPLGVD |
| | GSSTMTVESDYFIAYGVVERNDAAAAAKKKKAEGEAPALRVLGTKATFYNCTIEGG |
| | QGALYDQMGLHYFKSCTIRGTIDFIFGSAKSFYEDCTIVSVNNMEEIMTLPVAPPQ |
| | LDIHDNPIKVAPGEGGFSFKTCTITGEGQQIFLGRMGTPSIYSYTQTAKEVVPITY |
| | DKGNIFMPSNMTGRRCATFKCYGPGLEKIWHVKLRYAEAIYFLGTDFINGDSWILS |
| | IPPTDAETLLSV (SEQ ID NO: 25) |
| GA2-male | EELPLDFWVSALRGVGAKDVSTADGCTKKNKDTVLCSAQANTVINFINPINSEEQG |
| Mature (aa) | YRTIGESIANIPDDSTKRYILILSGGTVYREKVLVSKSKPFVTIRSDDPINPAIIV |
| | WNDTAATLGKDSKPLGVDGSSTMTVESDYFIAYGVVERNDAAAAAKKKKAEGEAPA |
| | LRVLGTKATFYNCTIEGGQGALYDQMGLHYFKSCTIRGTIDFIFGSAKSFYEDCTI |
| | VSVNNMEEIMTLPVAPPQLDIHDNPIKVAPGEGGFSFKTCTITGEGQQIFLGRMGT |
| | PSIYSYTQTAKEVVPITYDKGNIFMPSNMTGRRCATFKCYGPGLEKIWHVKLRYAE |
| | AIYFLGTDFINGDSWILSIPPTDAETLLSV (SEQ ID NO: 26) |
| GA2-male | ATGGAGAGCAGGAGGAAGAATCAACAGAAGACGAGCTACATTGTAATTTCCATCCA |
| Full-Length | GCTCCTAGCGGCCCTCCTACTCTTGGAGCTTGTGTCGCTAGTGCCGGGGGCGTCAT |
| (na) | GTGAGGAGTTGCCCTTGGATTTTTGGGTGAGTGCACTACGGGGGGTCGGGGCAAAG |
| | GACGTTTCAACAGCCGATGGATGCACCAAGAAGAATAAGGACACCGTGCTCTGCTC |
| | TGCCCAGGCTAACACGGTAACCAATTTCATCAACCCTACCAACTCTGAGGAACAAG |
| | GCTATAGGACCATCGGGGAGTCCATTGCTAACATCCCTGATGATAGCACCAAACGG |
| | TACATCCTCATCCTCAGCGGTGGCACCGTGTACCGAGAGAAGGTATTGGTGAGCAA |
| | AAGCAAGCCATTCGTCACCATAAGATCAGATGACCCCATCAACCCTGCCATCATTG |
| | TGTGGAACGACACTGCCGCCACCCTGGGGAAGGATAGCAAGCCCCTTGGAGTAGAT |
| | GGTAGTAGCACCATGACCGTAGAGTCCGACTACTTCATTGCTTATGGTGTCGTCTT |
| | TAGGAATGATGCTGCAGCAGCAGCGAAGAAGAAGAAGGCAGAAGGCGAGGCGCCAG |
| | CGTTGCGGGTGCTAGGAACAAAGGCAACCTTCTACAACTGCACAATTGAAGGTGGA |
| | CAAGGCGCCCTATATGACCAGATGGGGCTGCACTACTTCAAGTCCTGCACCATCAG |
| | GGGAACCATCGACTTCATCTTTGGCTCTGCCAAGTCCTTTTACGAGGACTGCACCA |
| | TTGTTTCCGTGAACAACATGGAGGAGATCATGACCTTGCCTGTGGCACCACCTCAA |
| | CTTGACATTCACGACAATCCAATCAAAGTTGCCCCAGGGGAGGGCGGCTTCTCCTT |
| | CAAGACATGTACCATCACTGGGGAAGGGCAACAAATCTTCCTCGGAAGGATGGGCA |
| | CGCCTTCCATCTACTCCTACACCCAGATTGCTAAGGAGGTTGTGCCCATAATCTAC |
| | GACAAAGGGAACATCTTCATGCCCAGTAATATGACTGGTAGACGCTGTGCCACTTT |
| | CAAGTGCTATGGACCTGGGTTAGAGAAAATATGGCACGTCAAACTTAGATACGCTG |
| | AAGCCATATACTTTCTTGGGACAGATTTTATCAACGGAGATTCATGGATCCTGTCC |
| | ATACCACCTACTGATGCTGAAACATTGCTATCAGTTTGA (SEQ ID NO: 27) |
| GA2-male | GAGGAGTTGCCCTTGGATTTTTGGGTGAGTGCACTACGGGGGGTCGGGGCAAAGGA |
| Mature (na) | CGTTTCAACAGCCGATGGATGCACCAAGAAGAATAAGGACACCGTGCTCTGCTCTG |
| | CCCAGGCTAACACGGTAACCAATTTCATCAACCCTACCAACTCTGAGGAACAAGGC |
| | TATAGGACCATCGGGGAGTCCATTGCTAACATCCCTGATGATAGCACCAAACGGTA |
| | CATCCTCATCCTCAGCGGTGGCACCGTGTACCGAGAGAAGGTATTGGTGAGCAAAA |
| | GCAAGCCATTCGTCACCATAAGATCAGATGACCCCATCAACCCTGCCATCATTGTG |
| | TGGAACGACACTGCCGCCACCCTGGGGAAGGATAGCAAGCCCCTTGGAGTAGATGG |
| | TAGTAGCACCATGACCGTAGAGTCCGACTACTTCATTGCTTATGGTGTCGTCTTTA |
| | GGAATGATGCTGCAGCAGCAGCGAAGAAGAAGAAGGCAGAAGGCGAGGCGCCAGCG |
| | TTGCGGGTGCTAGGAACAAAGGCAACCTTCTACAACTGCACAATTGAAGGTGGACA |
| | AGGCGCCCTATATGACCAGATGGGGCTGCACTACTTCAAGTCCTGCACCATCAGGG |
| | GAACCATCGACTTCATCTTTGGCTCTGCCAAGTCCTTTTACGAGGACTGCACCATT |
| | GTTTCCGTGAACAACATGGAGGAGATCATGACCTTGCCTGTGGCACCACCTCAACT |
| | TGACATTCACGACAATCCAATCAAAGTTGCCCCAGGGGAGGGCGGCTTCTCCTTCA |
| | AGACATGTACCATCACTGGGGAAGGGCAACAAATCTTCCTCGGAAGGATGGGCACG |
| | CCTTCCATCTACTCCTACACCCAGATTGCTAAGGAGGTTGTGCCCATAATCTACGA |
| | CAAAGGGAACATCTTCATGCCCAGTAATATGACTGGTAGACGCTGTGCCACTTTCA |
| | AGTGCTATGGACCTGGGTTAGAGAAAATATGGCACGTCAAACTTAGATACGCTGAA |
| | GCCATATACTTTCTTGGGACAGATTTTATCAACGGAGATTCATGGATCCTGTCCAT |
| | ACCACCTACTGATGCTGAAACATTGCTATCAGTTTGA (SEQ ID NO: 28) |
| GA2-female | MAAVGRSSLSLVLVMAAVIISGAGGERVPPAAKEPWPPAPHAVVVQQARLEECHRV |
| Full-Length | APACYTSIGQALAEAGKLVPKVKHRFVVLIKTGEYIEQVNITRRNVVLLGEGRGNT |
| (aa) | VISGNLSNLTGTAMVMTATVNVVADGFLAQNLTIRNEAGPKGRQAVALRSNSNRTV |
| | VFGCAIEGFEDSLYAENGVQVYLETDIYGTVDFIFGNAKAVFQRCRILVRRPIPGK |
| | HNVVTAQGCDNNMYENSGFVFHRCSVEADPNPWPVGQNLTGVETWLGGVGQGTRHQ |
| | (SEQ ID NO: 29) |
| GA2-female | ERVPPAAKEPWPPAPHAVVVQQARLEECHRVAPACYTSIGQALAEAGKLVPKVKHR |
| Mature | FVVLIKTGEYIEQVNITRRNVVLLGEGRGNTVISGNLSNLTGTAMVMTATVNVVAD |
| (aa) | GFLAQNLTIRNEAGPKGRQAVALRSNSNRTVVFGCAIEGFEDSLYAENGVQVYLET |
| | DIYGTVDFIFGNAKAVFQRCRILVRRPIPGKHNVVTAQGCDNNMYENSGFVFHRCS |
| | VEADPNPWPVGQNLTGVETWLGGVGQGTRHQ (SEQ ID NO: 30) |
| GA2-female | ATGGCAGCAGTTGGCAGATCGTCGCTGTCGCTCGTGCTGGTGATGGCGGCGGTGAT |
| Full- | CATCTCCGGCGCCGGTGGCGAGCGGGTGCCTCCTGCCGCAAAGGAGCCCTGGCCGC |
| Length | CGGCTCCGCATGCTGTGGTCGTACAGCAGGCGCGGCTGGAGGAGTGCCACCGAGTG |
| (na) | GCTCCGGCGTGCTACACCAGCATCGGACAAGCTCTGGCTGAAGCTGGCAAGCTTGT |
| | CCCCAAGGTGAAGCACCGTTTTGTTGTACTCATCAAGACCGGAGAGTACATAGAAC |
| | AGGTGAACATCACGAGACGGAACGTCGTCCTGCTCGGCGAGGGCAGAGGCAACACA |
| | GTAATTTCTGGCAACCTAAGCAACCTAACGGGGACGGCGATGGTGATGACGGCCAC |
| | CGTGAATGTTGTAGCCGACGGCTTTTTGGCCCAGAACCTGACGATCCGTAACGAGG |
| | CCGGGCCGAAAGGAAGGCAGGCGGTGGCTTTGAGGTCAAACTCCAACCGGACCGTC |
| | GTATTTGGCTGTGCTATAGAAGGCTTCGAAGATAGCCTGTACGCCGAAAATGGAGT |
| | CCAGGTGTACCTGGAGACGGACATATACGGGACCGTGGATTTTATATTCGGAAACG |
| | CGAAGGCGGTCTTCCAGCGTTGCCGCATCCTGGTGCGGCGGCCCATCCCCGGCAAG |
| | CACAACGTGGTGACGGCGCAGGGCTGCGACAACAATATGTACGAGAACTCCGGCTT |
| | CGTCTTCCACCGGTGCAGCGTCGAAGCTGACCCGAATCCATGGCCGGTGGGCCAGA |
| | ACCTGACCGGCGTGGAAACATGGCTGGGTGGCGTGGGACAGGGCACACGTCATCAA |
| | TGA (SEQ ID NO: 31) |
| GA2-female | GAGCGGGTGCCTCCTGCCGCAAAGGAGCCCTGGCCGCCGGCTCCGCATGCTGTGGT |
| Mature (na) | CGTACAGCAGGCGCGGCTGGAGGAGTGCCACCGAGTGGCTCCGGCGTGCTACACCA |
| | GCATCGGACAAGCTCTGGCTGAAGCTGGCAAGCTTGTCCCCAAGGTGAAGCACCGT |
| | TTTGTTGTACTCATCAAGACCGGAGAGTACATAGAACAGGTGAACATCACGAGACG |
| | GAACGTCGTCCTGCTCGGCGAGGGCAGAGGCAACACAGTAATTTCTGGCAACCTAA |
| | GCAACCTAACGGGGACGGCGATGGTGATGACGGCCACCGTGAATGTTGTAGCCGAC |
| | GGCTTTTTGGCCCAGAACCTGACGATCCGTAACGAGGCCGGGCCGAAAGGAAGGCA |
| | GGCGGTGGCTTTGAGGTCAAACTCCAACCGGACCGTCGTATTTGGCTGTGCTATAG |
| | AAGGCTTCGAAGATAGCCTGTACGCCGAAAATGGAGTCCAGGTGTACCTGGAGACG |
| | GACATATACGGGACCGTGGATTTTATATTCGGAAACGCGAAGGCGGTCTTCCAGCG |
| | TTGCCGCATCCTGGTGCGGCGGCCCATCCCCGGCAAGCACAACGTGGTGACGGCGC |
| | AGGGCTGCGACAACAATATGTACGAGAACTCCGGCTTCGTCTTCCACCGGTGCAGC |
| | GTCGAAGCTGACCCGAATCCATGGCCGGTGGGCCAGAACCTGACCGGCGTGGAAAC |
| | ATGGCTGGGTGGCGTGGGACAGGGCACACGTCATCAATGA (SEQ ID NO: 32) |
| GA2-male | GAATAAAACCAATTATTTGTTTTATTTTTCCATCGCTTATATATATATGCACCAGA |
| cDNA (na) | GATTTTTGTGTTACCCTTGCATATATGCATGCATGCATGTGGAATGAATCCAAGTC |
| | TAAAATTAGGCGCCTAATGGTGACCTTCCATATATTAGACACCTTAATTATTTGAA |
| | TTGGATTTTGGATGCAAACTAGCTTATTAGCTATCTATATATAACAAGATTAAAGA |
| | AGAAGACAGACAACCTAATTAAAGAATTTGAGGAGAGAAAGAAAGAAAGAGTGATC |
| | GTCATGGAGAGCAGGAGGAAGAATCAACAGAAGACGAGCTACATTGTAATTTCCAT |
| | CCAGCTCCTAGCGGCCCTCCTACTCTTGGAGCTTGTGTCGCTAGTGCCGGGGGCGT |
| | CATGTGAGGAGTTGCCCTTGGATTTTTGGGTGAGTGCACTACGGGGGGTCGGGGCA |
| | AAGGACGTTTCAACAGCCGATGGATGCACCAAGAAGAATAAGGACACCGTGCTCTG |
| | CTCTGCCCAGGCTAACACGGTAACCAATTTCATCAACCCTACCAACTCTGAGGAAC |
| | AAGGCTATAGGACCATCGGGGAGTCCATTGCTAACATCCCTGATGATAGCACCAAA |
| | CGGTACATCCTCATCCTCAGCGGTGGCACCGTGTACCGAGAGAAGGTATTGGTGAG |
| | CAAAAGCAAGCCATTCGTCACCATAAGATCAGATGACCCCATCAACCCTGCCATCA |
| | TTGTGTGGAACGACACTGCCGCCACCCTGGGGAAGGATAGCAAGCCCCTTGGAGTA |
| | GATGGTAGTAGCACCATGACCGTAGAGTCCGACTACTTCATTGCTTATGGTGTCGT |
| | CTTTAGGAATGATGCTGCAGCAGCAGCGAAGAAGAAGAAGGCAGAAGGCGAGGCGC |
| | CAGCGTTGCGGGTGCTAGGAACAAAGGCAACCTTCTACAACTGCACAATTGAAGGT |
| | GGACAAGGCGCCCTATATGACCAGATGGGGCTGCACTACTTCAAGTCCTGCACCAT |
| | CAGGGGAACCATCGACTTCATCTTTGGCTCTGCCAAGTCCTTTTACGAGGACTGCA |
| | CCATTGTTTCCGTGAACAACATGGAGGAGATCATGACCTTGCCTGTGGCACCACCT |
| | CAACTTGACATTCACGACAATCCAATCAAAGTTGCCCCAGGGGAGGGCGGCTTCTC |
| | CTTCAAGACATGTACCATCACTGGGGAAGGGCAACAAATCTTCCTCGGAAGGATGG |
| | GCACGCCTTCCATCTACTCCTACACCCAGATTGCTAAGGAGGTTGTGCCCATAATC |
| | TACGACAAAGGGAACATCTTCATGCCCAGTAATATGACTGGTAGACGCTGTGCCAC |
| | TTTCAAGTGCTATGGACCTGGGTTAGAGAAAATATGGCACGTCAAACTTAGATACG |
| | CTGAAGCCATATACTTTCTTGGGACAGATTTTATCAACGGAGATTCATGGATCCTG |
| | TCCATACCACCTACTGATGCTGAAACATTGCTATCAGTTTGAATATATAGAATCCT |
| | CAGAGATCATCAAAGATCATCATCTACCACTAATTAATAATGAGCCAAATTAAATA |
| | AGTTTTGCGTATATATTTTATCCCTATCTATAAATTATGCATGTGTTGTAAACAAG |
| | TCGTGGATGGGCTTCATTTGTTCTCGCGATTGAAAAATAACAAATAAACAAAGTAG |
| | AAAAGAGGATGAACCAGAAGTCTTGTTCAGCAAGAAGGAAGGAGGAATGACGATAT |
| | TTCCCATGTGATACTTGTAAATCATATGTGTAAACTTCGTGGGCGTACTTGTAATT |
| | TCATACGAAGTTGTACCTCTCGCCTATAAATAGAGTAACAGTGTCATGCATAAATT |
| | CACCTTTCGAGAGCTCAAAGCTTCATTTAGCTCAGCCTTCGAAGTACCTTTGAGCT |
| | ATCACTTGTATGAAGCCGAAGGTATGTTTGTAATTGTTTGTTATATTGCAAGGAGT |
| | GAAATAATAAGTGTGGAAGCATGAAGATGAGTAACATGTCTTGCTCGTAACTGTTT |
| | ACATTTCATCCTTGTTAATATATAAGCATTTGTGCCC (SEQ ID NO: 33) |
| GA2-female | CGCGGCCAGCCTATATATATATGGTATCGGACCTGAGGCTGCTGGAGTGCTAGTTG |
| cDNA (na) | AGCTTGAGCAAGGTTGAGTGAAGAGAGTGACACACGCACACGACGCACTACGTTGG |
| | CCCGCTACGAGCTGCAACTGGATCATGGCAGCAGTTGGCAGATCGTCGCTGTCGCT |
| | CGTGCTGGTGATGGCGGCGGTGATCATCTCCGGCGCCGGTGGCGAGCGGGTGCCTC |
| | CTGCCGCAAAGGAGCCCTGGCCGCCGGCTCCGCATGCTGTGGTCGTACAGCAGGCG |
| | CGGCTGGAGGAGTGCCACCGAGTGGCTCCGGCGTGCTACACCAGCATCGGACAAGC |
| | TCTGGCTGAAGCTGGCAAGCTTGTCCCCAAGGTGAAGCACCGTTTTGTTGTACTCA |
| | TCAAGACCGGAGAGTACATAGAACAGGTGAACATCACGAGACGGAACGTCGTCCTG |
| | CTCGGCGAGGGCAGAGGCAACACAGTAATTTCTGGCAACCTAAGCAACCTAACGGG |
| | GACGGCGATGGTGATGACGGCCACCGTGAATGTTGTAGCCGACGGCTTTTTGGCCC |
| | AGAACCTGACGATCCGTAACGAGGCCGGGCCGAAAGGAAGGCAGGCGGTGGCTTTG |
| | AGGTCAAACTCCAACCGGACCGTCGTATTTGGCTGTGCTATAGAAGGCTTCGAAGA |
| | TAGCCTGTACGCCGAAAATGGAGTCCAGGTGTACCTGGAGACGGACATATACGGGA |
| | CCGTGGATTTTATATTCGGAAACGCGAAGGCGGTCTTCCAGCGTTGCCGCATCCTG |
| | GTGCGGCGGCCCATCCCCGGCAAGCACAACGTGGTGACGGCGCAGGGCTGCGACAA |
| | CAATATGTACGAGAACTCCGGCTTCGTCTTCCACCGGTGCAGCGTCGAAGCTGACC |
| | CGAATCCATGGCCGGTGGGCCAGAACCTGACCGGCGTGGAAACATGGCTGGGTGGC |
| | GTGGGACAGGGCACACGTCATCAATGATACCACGAAGAGCGTGAGATACATGGAGT |
| | ATGCCAACACAGGTCCCGGCGCCGACACCACCCACCGCGTCGACTGGGAGGGCGTC |
| | CATGTTCTCCACGACCCCGCCCAGGTAGCCAAATACACGATAGATGCCTTCATATC |
| | GGGTAAGGAGTGGATTCCCCATCAGATCCCGTATGACCATGAGGTCCCCAGCGGGC |
| | GCGGCGCCATCATCGTCAACTAGCCGGCCGGCGGCCAGTCTCCCTCCCATCATCTG |
| | CGCGCTTCAATTCATCTGCTCCCTCCTG (SEQ ID NO: 34) |

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. An isolated nucleic acid(s) comprising a polynucleotide sequence selected from:
(a) a polynucleotide sequence encoding a polypeptide comprising the amino acid sequence of SEQ ID NO:2 (Tcb1-m mature aa);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as pectin methylesterase (PME) activity or the ability to bind ZmPME10-1 having the amino acid sequence of SEQ ID NO:21 (ZmPME10-1, mat);
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(e) a polynucleotide sequence encoding a polypeptide comprising the amino acid sequence of SEQ ID NO:6 (Tcb1-f);
(f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
(h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity;
(i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na);
(j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na);
(k) a polynucleotide sequence encoding a polypeptide comprising the amino acid sequence of SEQ ID NO:25 (GA2-m, fl) or SEQ ID NO:26 (GA2-m, mat);
(l) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m, mat);
(m) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m, mat), wherein the polypeptide has at least one GA2-m biological activity such as PME activity;
(n) a polynucleotide sequence encoding a polypeptide comprising an amino acid sequence that is at least 98%, 98.5%, 99%, or 99.5% identical to the amino acid sequence of SEQ ID NO:26 (GA2-m, mat), wherein the polypeptide has at least one GA2-m biological activity such as PME activity;
(o) a polynucleotide sequence encoding a polypeptide comprising the amino acid sequence of SEQ ID NO:29 (GA2-f, fl) or SEQ ID NO:30 (GA2-f, mat);
(p) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f, mat), wherein the polypeptide has GA2-f biological activity such as PME activity;
(q) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO: NO:30 (GA2-f, mat), wherein the polypeptide has at least one GA2-f biological activity such as PME activity;
(r) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity; or
(s) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na); and/or
(t) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); or
(u) the polynucleotide sequence of SEQ ID NO:9 (Tcb1-m, prom);
(v) a polynucleotide sequence comprising at least 100 consecutive nucleotides of the polynucleotide sequence of SEQ ID NO:9;
(w) a polynucleotide sequence that hybridizes to the complete complement of the polynucleotide sequence of (u) or (v) under stringent hybridization conditions; and
(x) a polynucleotide sequence having at least 95% sequence identity to the polynucleotide sequences of (u) or (v);
(y) the polynucleotide sequence of SEQ ID NO:12 (GA1-f, prom);
(z) a polynucleotide sequence comprising at least 100 consecutive polynucleotides of nucleotides 1 to 800 of SEQ ID NO:12;
(aa) a polynucleotide sequence that hybridizes to the complete complement of the polynucleotide sequence of (y) or (z) under stringent hybridization conditions; and
(ab) a polynucleotide sequence having at least 95% sequence identity to the polynucleotide sequences of (y) or (z) or
(ac) the polynucleotide sequence of SEQ ID NO:12 (GA1-f, prom);
(ad) a polynucleotide sequence comprising at least 100 consecutive polynucleotides of nucleotides 1 to 800 of SEQ ID NO:12;
(ae) a polynucleotide sequence that hybridizes to the complete complement of the polynucleotide sequence of (ac) or (ad) under stringent hybridization conditions; and
(af) a polynucleotide sequence having at least 95% sequence identity to the polynucleotide sequences of (ac) or (ad).

2. The nucleic acid(s) of claim 1, which:
(i) encodes the polypeptide of any one of (a)-(d), but does not encode the polypeptide of any one of (e)-(h);
(ii) encodes the polypeptide of any one of (e)-(h), but does not encode the polypeptide of any one of (a)-(d);
(iii) encodes the polypeptide of any one of (a)-(d), and the polypeptide of any one of (e)-(h);
(iv) comprises the polynucleotide sequence of (i) but does not comprises the polynucleotide sequence of (j);
(v) comprises the polynucleotide sequence of (j) but does not comprises the polynucleotide sequence of (i);
(vi) comprises the polynucleotide sequence of (i) and the polynucleotide sequence of (j)
(vii) encodes the polypeptide of any one of (k)-(n), but does not encode the polypeptide of any one of (o)-(r);
(viii) encodes the polypeptide of any one of (o)-(r), but does not encode the polypeptide of any one of (k)-(n);
(ix) encodes the polypeptide of any one of (k)-(n), and the polypeptide of any one of (o)-(r);
(x) comprises the polynucleotide sequence of (i) but does not comprises the polynucleotide sequence of (t);
(xi) comprises the polynucleotide sequence of (j) but does not comprises the polynucleotide sequence of (s); or
(xii) comprises the polynucleotide sequence of (s) and the polynucleotide sequence of (t).

3.-4. (canceled)

5. The nucleic acid(s) of claim 1, which further comprises:
(a) a nucleic acid comprising a polynucleotide sequence encoding a polypeptide comprising the amino acid sequence of SEQ ID NO:21 (ZmPME10-1, mat);
(b) a polynucleotide sequence encoding a polypeptide comprising a fragment of a protein having the amino acid sequence of at least about 150, 200, 250 300, 350, 400, 450, 500, 550, 600, 650, 655, 660, 665, 670, 657, 676, 677, 678, 679, or 680, contiguous amino acids of SEQ ID NO:21, wherein the polypeptide has ZmPME10-1 biological activity such as the ability to bind Tcb1-m (SEQ ID NO:2);
(c) a nucleic acid comprising a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:21 (ZmPME10-1), wherein the polypeptide has ZmPME10-1 biological activity such as the ability to bind Tcb1-m (SEQ ID NO:2); and/or
(d) a nucleic acid comprising a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 or SEQ ID NO:4).

6. The nucleic acid(s) of claim 1, which further comprises:
(a1) a polynucleotide sequence encoding a polypeptide comprising the amino acid sequence of SEQ ID NO:13 (GA1-m, fl) or SEQ ID NO:14 (GA1-m, mat);
(b1) a polynucleotide sequence encoding a polypeptide comprising a fragment of a protein having the amino acid sequence of at least about 150, 200, 225, 250 275, 300, 325, 350, 351, 352, 353, or 354, contiguous amino acids of SEQ ID NO:14, wherein the polypeptide has at least one GA1-m biological activity such as PME activity or the ability to bind ZmPME10-1 having the amino acid sequence of SEQ ID NO:21;
(c1) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:14, wherein the polypeptide has at least one GA1-m biological activity such as PME activity or the ability to bind ZmPME10-1 having the amino acid sequence of SEQ ID NO:21; and/or
(d1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:15 or SEQ ID NO:16 (GA1-m, fl na); and/or
(a2) a polynucleotide sequence encoding a polypeptide comprising the amino acid sequence of of SEQ ID NO:13 (GA1-m, fl) or SEQ ID NO:14 (GA1-m, mat);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:18, wherein the polypeptide has at least one GA1-f biological activity such as PME activity;
(c2) a polynucleotide sequence encoding a polypeptide comprising an amino acid sequence having a total of one, two, three, four, five, six, seven, eight, nine, ten, fifteen, twenty, twenty five, or thirty, or fewer than thirty, fewer than twenty five, fewer than twenty, fewer than fifteen, fewer than ten, fewer than nine, fewer than eight, fewer than seven, fewer than six, fewer than five, fewer than four, fewer than three, or fewer than two amino acid substitutions, deletions, and/or insertions from a reference amino acid sequence of SEQ ID NO:18, wherein the polypeptide has at least one GA1-f biological activity such as PME activity; and/or
(d2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:12, SEQ ID NO:19, or SEQ ID NO:20 (GA1-f prom, mat, full-length na).

7. The nucleic acid(s) of claim 1, which is operably linked to one, or more than one, promoter, optionally wherein the one, or more than one, promoter is a constitutive promoter or an inducible promoter.

8. The nucleic acid(s) of claim 7, which is operably linked to one, or more than one, endogenous promoter or heterologous promoter, optionally wherein the nucleic acid(s) is operably linked to one, or more one, male specific promoter (e.g., a pollen-and pollen-tube specific promoter) or one, or more than one, female specific promoter (e.g., a corn silk specific promoter), or a fragment thereof.

9. The nucleic acid(s) of claim 7 wherein the promoter comprises at least 200 nucleotides of a Tcb1-m promoter, a GA1-m promoter, Tcb1-f promoter, or a GA1-f promoter.

10. The nucleic acid(s) of claim 5, wherein the one, or more than one promoter comprises a polynucleotide sequence selected from:
(a) a polynucleotide sequence comprising at least about 50, 60, 75, 100, 125, 150, 175, 200, 225, 260, 275, 300, 325, 350, 375, 400 or more nucleotides of SEQ ID NO:9 (Tcb1-m, prom);
(b) a polynucleotide sequence that is at least 85%, 90%, 95%, 96%, 97%, 98%, or 99%, identical to the polynucleotide sequence of SEQ ID NO:9 (Tcb1-m, prom);
(c) a polynucleotide sequence comprising at least about 50, 60, 75, 100, 125, 150, 175, 200, 225, 260, 275, 300, 325, 350, 375, 400 or more nucleotides of a GA1-m promoter;
(d) a polynucleotide sequence comprising at least about 50, 60, 75, 100, 125, 150, 175, 200, 225, 260, 275, 300, 325, 350, 375, 400 or more nucleotides of a ZmPME10-1 promoter;
(e) a polynucleotide sequence comprising at least about 50, 60, 75, 100, 125, 150, 175, 200, 225, 260, 275, 300, 325, 350, 375, 400 or more nucleotides of a Tcb1-f promoter;
(f) a polynucleotide sequence comprising at least about 50, 60, 75, 100, 125, 150, 175, 200, 225, 260, 275, 300, 325, 350, 375, 400 or more nucleotides of SEQ ID NO:12 (GA1-f, prom); and/or
(g) a polynucleotide sequence that is at least 85%, 90%, 95%, 96%, 97%, 98%, or 99%, identical to the polynucleotide sequence of SEQ ID NO:12 (GA1-f, prom).

11. An expression cassette or vector comprising the nucleic acid(s) of claim 1, optionally wherein the vector expresses a polypeptide with Tcb1-m, Tcb1-f, GA1-m, GA1-f, GA2-m, GA2-f, or ZmPME10-1, biological activity, such as PME activity.

12. A host cell comprising the nucleic acid(s) of claim 1.

13. The host cell of claim 12, wherein the nucleic acid(s) is integrated into the DNA of the host cell.

14. The host cell of claim 12, wherein the host cell is a bacterial, fungal, insect, animal, or plant cell.

15. (canceled)

16. The host cell of claim 1, wherein the host cell is a monocot.

17. The host cell of claim 1, wherein the host cell is a maize cell.

18. The host cell of claim 1, wherein the host cell is a wheat cell, a rice cell, a barley cell, an oat cell, a proso millet cell, a rye cell, a turfgrass cell, a fescue cell, a sorghum cell, a sugarcane cell, or an algal cell, or wherein the host cell is a dicot, optionally wherein the cell is a soybean cell, canola cell, sunflower cell, a sugar beet cell, a quinoa cell, an alfalfa cell, or a cotton cell.

19.-20. (canceled)

21. A transgenic plant stably transformed with the isolated nucleic acid(s) of claim 1, optionally wherein the transgenic plant is stably transformed with the nucleic acid of any one of 1(a)-1(d), 1(e)-1(f), 1(k)-1(n), 1(o)-1(p).

22.-23. (canceled)

24. A method of conferring self-incompatibility or male sterility to a plant, wherein the method of conferring self-incompatibility comprises:
stably transforming a plant cell with an isolated nucleic(s) acid comprising a polynucleotide sequence selected from the group:
(a1) a polynucleotide sequence encoding a polypeptide comprising the amino acid sequence of SEQ ID NO:6 (Tcb1-f);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such PME activity;
(d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity; and/or
(e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
an isolated nucleic(s) acid comprising a polynucleotide sequence selected from the group:
(a2) a polynucleotide sequence encoding a polypeptide comprising the amino acid sequence of SEQ ID NO:6 (Tcb1-f);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
(d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity; and/or
(e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); or
(f1) a polynucleotide sequence encoding a polypeptide comprising the amino acid sequence of SEQ ID NO:29 (GA2-f, fl) or SEQ ID NO:30 (GA2-f, mat);
(g1) a polynucleotide sequence encoding a polypeptide comprising a fragment of a protein having the amino acid sequence of at least about 50, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
(h1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such PME activity;
(i1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity; and/or
(j1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
an isolated nucleic(s) acid comprising a polynucleotide sequence selected from the group:
(f2) a polynucleotide sequence encoding a polypeptide comprising the amino acid sequence of SEQ ID NO:29 (GA2-f, fl) or SEQ ID NO:30 (GA2-f, mat);
(g2) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
(h2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
(i2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity; and/or
(j2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing the transgenic plant from the plant cell comprising the isolated nucleic acid, and expressing the polynucleotide sequence in the plant, optionally, wherein the plant is not teosinte or does not express Tcb1-m; and wherein the method of conferring self-incompatibility comprises stably transforming a plant cell with an isolated nucleic acid(s) comprising a polynucleotide sequence selected from:
(a) a polynucleotide sequence encoding a polypeptide comprising the amino acid sequence of SEQ ID NO:6 (Tcb1-f);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
(d) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
(e) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na);
(f) a polynucleotide sequence encoding a polypeptide comprising the amino acid sequence of SEQ ID NO:29 (GA2-f, fl) SEQ ID NO:30 (GA2-f, mat);
(g) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
(h) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
(i) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity; and/or
(j) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing a plant from the transformed plant cell comprising the isolated nucleic acid, and expressing the polynucleotide sequence in the plant; optionally, wherein the polynucleotide sequence is operably associated with one, or more than one, constitutive or inducible promoter, or one, or more than one, male-specific promoter (e.g., a pollen and a pollen-tube specific promoter).

25. The method of claim 24, which further comprises using a Tcb1-m plant comprising a polynucleotide sequence encoding a polypeptide comprising the amino acid sequence of SEQ ID NO:2 (Tcb1-m) from a separate plant line (either a natural Tcb1-m line or a transgenic Tcb1-m line) to force cross-pollination.

26. (canceled)

27. A method of creating an isolated breeding population of plants or a substantially homogeneous population of plants of a predetermined hybrid variety of a crop which is capable of undergoing both self-pollination and cross-pollination, wherein, the method of creating an isolated breeding population comprises:
stably transforming a plant cell with an isolated nucleic acid(s) comprising a polynucleotide sequence selected from:
(a) a polynucleotide sequence encoding a polypeptide comprising the amino acid sequence of SEQ ID NO:2 (Tcb1-m);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as PME activity or the ability to bind ZmPME10-1 having the amino acid sequence of SEQ ID NO:21 (ZmPME10-1, mat);
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(e) a polynucleotide sequence encoding a polypeptide comprising the amino acid sequence of SEQ ID NO:6 (Tcb1-f, mat);
(f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
(g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
(h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
(i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na);
(j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na);
(k) a polynucleotide sequence encoding a polypeptide comprising the amino acid sequence of SEQ ID NO:26 (GA2-m, mat);
(l) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m), wherein the polypeptide has at least one GA2-m biological activity such as PME activity;
(m) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m), wherein the polypeptide has at least one GA2-m biological activity such as PME activity;
(n) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m), wherein the polypeptide has at least one GA2-m biological activity such as PME activity;
(o) a polynucleotide sequence encoding a polypeptide comprising the amino acid sequence of SEQ ID NO:29 (GA2-f, fl) or SEQ ID NO:30 (GA2-f, mat);
(p) a polynucleotide sequence encoding a polypeptide comprising a fragment of a protein having the amino acid sequence of at least about 0, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has at least one GA2-f biological activity such as PME activity;
(q) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
(r) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
(s) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na); and/or
(t) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter); and
wherein the method for forming a substantially homogeneous population of plants of a predetermined hybrid variety of a crop which is capable of undergoing both self-pollination and cross-pollination comprises stably transforming a plant cell with an isolated nucleic(s) acid comprising a polynucleotide sequence selected from the group:
(a1) a polynucleotide sequence encoding a polypeptide comprising the amino acid sequence of SEQ ID NO:6 (Tcb1-f);
(b1) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such PME activity;
(d1) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
(e1) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); and
an isolated nucleic(s) acid comprising a polynucleotide sequence selected from the group:
(a2) a polynucleotide sequence encoding a polypeptide comprising the amino acid sequence of SEQ ID NO:6 (Tcb1-f);
(b2) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has Tcb1-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has Tcb1-f biological activity such as PME activity;
(d2) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has Tcb1-f biological activity such as PME activity; and
(e2) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na); or
stably transforming a plant cell with an isolated nucleic(s) acid comprising a polynucleotide sequence selected from the group:
(a1) a polynucleotide sequence encoding a polypeptide comprising the amino acid sequence of SEQ ID NO:29 (GA2-f, fl) or SEQ ID NO:30 (GA2-f, mat);
(b1) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has GA2-f biological activity such as PME activity;
(c1) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such PME activity;
(d1) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity; and
(e1) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing a plant from the transformed plant cell comprising the isolated nucleic acid, and expressing the polynucleotide sequence in the plant, optionally, wherein the plant does not express Tcb1-m; or stably transforming a plant cell with an isolated nucleic acid(s) comprising a polynucleotide sequence selected from an isolated nucleic(s) acid comprising a polynucleotide sequence selected from the group:
(a2) a polynucleotide sequence encoding a polypeptide comprising the amino acid sequence of SEQ ID NO:29 (GA2-f, fl) or SEQ ID NO:30 (GA2-f, mat);
(b2) a polynucleotide sequence encoding a polypeptide comprising a fragment of a protein having the amino acid sequence of at least about 50, 75, 100, 125, 130, 135, 140, 145, 150, 175, 200, 205, 210, 215, 220, 225, 230, 235, 240, 245, 246, 247, 248, 249, 250, 251, 252, 253, or 254 contiguous amino acids of SEQ ID NO:30 (GA2-f), wherein the polypeptide has GA2-f biological activity such as PME activity;
(c2) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has GA2-f biological activity such as PME activity;
(d2) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has GA2-f biological activity such as PME activity; and
(e2) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing a plant from the transformed plant cell comprising the isolated nucleic acid, and expressing the polynucleotide sequence in the plant, optionally, wherein the plant does not express GA2-m.

28. (canceled)

29. A method of overcoming species barriers to enable fertilization and transfer of traits between species, the method comprising stably transforming a plant cell with an isolated nucleic acid encoding a polypeptide selected from the group:
stably transforming a plant cell with an isolated nucleic acid(s) comprising a polynucleotide sequence selected from:
(a) a polynucleotide sequence encoding a polypeptide comprising the amino acid sequence of SEQ ID NO:2 (Tcb1-m);
(b) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 351, 352, 353, 354, or 355 contiguous amino acids of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(c) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(d) a polynucleotide sequence encoding a polypeptide comprising an amino acid sequence that is at least 98%, 98.5%, 99%, or 99.5% identical to the amino acid sequence of SEQ ID NO:2 (Tcb1-m), wherein the polypeptide has at least one Tcb1-m biological activity such as PME activity or the ability to bind ZmPME10-1 having the amino acid sequence of SEQ ID NO:21;
(e) a polynucleotide sequence encoding a polypeptide comprising the amino acid sequence of SEQ ID NO:6 (Tcb1-f);
(f) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, 365, 366, 367, or 368, contiguous amino acids of SEQ ID NO:6 (Tcb1-f), wherein the polypeptide has at least one Tcb1-f biological activity such as PME activity;
(g) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:6 (Tcb1-f), wherein the polypeptide has at least one Tcb1-f biological activity such as PME activity;
(h) a polynucleotide sequence encoding a polypeptide comprising an amino acid sequence that is at least 98%, 98.5%, 99%, or 99.5% identical to the amino acid sequence of SEQ ID NO:6 (Tcb1-f), wherein the polypeptide has at least one Tcb1-f biological activity such as PME activity;
(i) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:3 (Tcb1-m, fl na); and/or
(j) a polynucleotide sequence comprising at least about 10, 15, 20, 25, 30, 35, 40, 45, 50, 75, 100, 125, 150, 175, 200, 250 300, 350, 400, 450, 500, 600, 700, 800, 900, 1,000, 2,000, 3,000, 4,000, or 5,000 contiguous nucleotides of the sequence of SEQ ID NO:7 (Tcb1-f, fl na);
(k) a polynucleotide sequence encoding a polypeptide comprising the amino acid sequence of SEQ ID NO:26 (GA2-m, mat);
(l) a polynucleotide sequence encoding a polypeptide comprising a fragment of a protein having the amino acid sequence of at least about 150, 200, 250 300, 325, 330, 335, 340, 341, 342, 343, 344, 345, 346, 347, 348, 349, 350, 355, 360, 361, 362, 363, 364, or 365 contiguous amino acids of SEQ ID NO:26 (GA2-m, mat);
(m) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:26 (GA2-m), wherein the polypeptide has at least one GA2-m biological activity such as PME activity;
(n) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:26 (GA2-m), wherein the polypeptide has at least one GA2-m biological activity such as PME activity;
(o) a polynucleotide sequence encoding a polypeptide comprising the amino acid sequence of SEQ ID NO:29 (GA2-f, fl) or SEQ ID NO:30 (GA2-f, mat);
(p) a polynucleotide sequence encoding a polypeptide comprising a fragment of a protein having the amino acid sequence of at least about 50, 75, 100, 125, 150, 175, 200, 205, 210, 215, 220, 225, 230, 235, 240, 245, 246, 247, 248, 249, 250, 251, 252, 253, or 254 contiguous amino acids of SEQ ID NO: SEQ ID NO:30 (GA2-f, mat), wherein the polypeptide has GA2-f biological activity such as PME activity;
(q) a polynucleotide sequence encoding a polypeptide comprising an amino acid sequence having a total of one, two, three, four, five, six, or seven, or fewer than eight, fewer than seven, fewer than six, fewer than five, fewer than four, fewer than three, or fewer than two amino acid substitutions, deletions, and/or insertions from a reference amino acid sequence of SEQ ID NO:30 (GA2-f), wherein the polypeptide has at least one GA2-f biological activity such as PME activity;
(r) a polynucleotide sequence encoding a polypeptide comprising an amino acid sequence that is at least 75%, 80%, 85%, 90%, 95%, 96%, 97%, 98%, 98.5%, 99%, or 99.5% identical to the amino acid sequence of SEQ ID NO:30 (GA2-f), wherein the polypeptide has at least one GA2-f biological activity such as PME activity;
(s) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 850, 900, 950, 1,000, 1,100, or 1,200 contiguous nucleotides of the sequence of SEQ ID NO:27 (GA2-m, fl na); and/or
(t) a polynucleotide sequence comprising at least about 10, 15, 20, 25, 30, 35, 40, 45, 50, 75, 100, 125, 150, 175, 200, 250 300, 350, 400, 450, 500, 550, 600, 650, 700, 750, 800, 820, or 840 contiguous nucleotides of the sequence of SEQ ID NO:31 (GA2-f, fl na); and
regenerating or developing a plant from the transformed plant cell comprising the isolated nucleic acid, and expressing the polynucleotide sequence in the plant, optionally, wherein the polynucleotide sequence is operably associated with one, or more than one, constitutive or inducible promoter, or one, or more than one, male-specific promoter (e.g., a pollen and a pollen-tube specific promoter) or one or more than one female-specific promoter (e.g., a corn silk specific promoter).

30. (canceled)

31. The method of claim 29, which further comprises using a GA2-m plant comprising a polynucleotide sequence encoding a polypeptide comprising the amino acid sequence of SEQ ID NO:26 (GA2-m, mat) from a separate plant line (either a natural GA2-m line or a transgenic GA2-m line) to force cross-pollination.

32.-38. (canceled)
